(19) United States
(12) Patent Application Publication
(10) Pub. No.: US 2006/0172418 A1
(43) Pub. Date: Aug. 3, 2006

(54) Hypercoiling polymers and their use in cellular delivery

(76) Inventors: Nigel K.H. Slater, Cambridge (GB); Mark E. Eccleston, Cambridge (GB)

Correspondence Address: NIXON & VANDERHYE, PC, 901 NORTH GLEBE ROAD, 11TH FLOOR, ARLINGTON, VA, 22203, US

(21) Appl. No.: 10/537,543

(22) PCT Filed: Dec. 2, 2003
(86) PCT No.: PCT/GB03/05262

§ 371 Date: Jun. 3, 2005

(30) Foreign Application Priority Data
Dec. 6, 2002 (GB) 0228525.2

(51) Int. Cl. C12N 15/87

(52) U.S. Cl. 435/455

(57) ABSTRACT

This invention pertains to certain hypercoiling polymers, and their use for the delivery of a payload into a living cell, e.g., into the nucleus of a living cell, which polymer incorporates, or is otherwise associated with, said payload. The payload may be, for example, a therapeutic payload, such as a drug, etc.; a diagnostic payload, such as a detectable label, such as a fluorophore, etc. In preferred embodiments, the hypercoiling polymers are biocompatible; biodegradable; comprise amide linkages; and/or are pseudo-proteins. The present invention also pertains to certain hypercoiling polymers; certain hypercoiling carrier polymers, which incorporate a payload; and certain hypercoiling carrier polymers, otherwise associated with a payload; which are suitable for use in such methods; and methods of diagnosis, treatment, imaging, etc., using such polymers.

### RELATED APPLICATION

This application is related to, and where permitted by law, claims priority to, United Kingdom patent application GB 0228525.2 filed 6 Dec. 2002; the contents of which are incorporated herein by reference in their entirety.

### TECHNICAL FIELD

This invention pertains generally to the field of polymers, and the biological and medical applications of polymers. More particularly, the present invention pertains to certain hypercoiling polymers, and their use for the delivery of a payload into a living cell, e.g., into the nucleus of a living cell, which polymer incorporates, or is otherwise associated with, said payload. The payload may be, for example, a therapeutic payload, such as a drug, etc.; a diagnostic payload, such as a detectable label, such as a fluorophore, etc.; or both. In preferred embodiments, the hypercoiling polymers are biocompatible; biodegradable; comprise amide linkages; and/or are pseudo-proteins. The present invention also pertains to hypercoiling polymers; hypercoiling carrier polymers, which incorporate a payload; and hypercoiling carrier polymers, otherwise associated with a payload; which are suitable for use in such methods; and methods of diagnosis, treatment, imaging, etc., using such polymers.

### BACKGROUND

Throughout this specification, including any claims which follow, unless the context requires otherwise, the word “comprise,” and variations such as “comprises” and “comprising,” will be understood to imply the inclusion of a stated integer or step or group of integers or steps, but not the exclusion of any other integer or step or group of integers or steps.

It must be noted that, as used in the specification and any appended claims, the singular forms “a,” “an,” and “the” include plural referents unless the context clearly dictates otherwise. Thus, for example, reference to “a carrier polymer” includes mixtures of two or more such carrier polymers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Biopolymers for Cell Delivery

Biopolymers, and their potential uses, have been the subject of a great many studies.

Matthews et al., 1996, provides a detailed review of the biological applications of macromolecules, including methods of delivery and imaging.

Ringsdorf, 1975, provides an early discussion of the use of polymers as pharmacologically active agents.

Tirrell et al., 1996 provides a brief review of biopolymers, such as proteins, polyesters, polysaccharides, and polynucleotides, and methods for their synthesis.

Angelova et al., 1999, provides a brief review of polymeric biomaterials, including their properties, their uses, and the relationships between the two.

Hoffman et al., 1992 and Hoffman et al., 2000, provide a brief review of stimulus-responsive (“smart”) polymer systems, and particularly polymer-biomolecule conjugates, and specifically polymer-protein conjugates.

Polymers having amide, peptide, and/or pseudo-peptide linkages have received much attention because of their potentially improved biodegradability and biocompatability.

Saotome et al., 1967, describe polyamides prepared from L-lysine and adipic acid (as adipyl choride) and from a novel diamine (prepared from L-lysine and adipyl chloride) with adipyl chloride.

Li et al., 1989, describe the synthesis of poly(iminocarbonates) (see FIG. 1 therein), and more particularly, the synthesis of poly(Bisphenol A-iminocarbonate) from Bisphenol A and Bisphenol A dicyanate. It was suggested that such polymers may find medical applications as biomaterials.

Domb et al., 1990, describe methods of synthesis of biodegradable poly(amide anhydride)s and poly(amide ester)s based on naturally occurring amino acids. Biodegradable polymers containing an active agent release that agent in vivo as they are hydrolytically degraded by body fluids. When such polymers are used for the delivery of drugs, it is desirable that the polymers themselves be non-toxic and degrade into non-toxic products. To minimize their toxicity and the toxicity of the their degradation products, polymers were synthesized from naturally occurring compounds, such as polyesters derived from lactic or glycolic acid, and polypeptides derived from amino acids.

Boustta et al., 1991, describe methods of synthesis of aliphatic polyamides bearing pendant carboxylic acid and tertiary alcohol groups, from di-acyl chloride activated citric acid and carboxy-protected L-lysine. Such water-soluble functional polymers were suggested to be useful as carriers for the design of macromolecular prodrugs.

Mungara et al., 1993 and Mungara et al., 1994, describe methods of synthesis of polyamides containing a tyrosine-leucine linkage, which provides an enzyme targeted cleavage point. It was suggested that polyamides derived from the dipeptide would be biodegradable.

Fiordeliso et al., 1994, describe the design, synthesis, and characterization of tyrosine-containing polyarylates. It is suggested that such polymers (see FIGS. 1 and 2 therein), as examples of aliphatic polyarylates (as distinct from aromatic polyesters), are biodegradable and are useful in biomedical applications.

Vinyl Polymers

Thomas et al., 1992 report that the hydrophobic polyelectrolyte poly(2-ethylacrylic acid) solubilizes lipid membranes in a pH-dependent manner. The authors exploited that phenomenon to prepare lipid vesicles that release their contents in response to pH, light, or glucose.

Thomas et al., 1994, describe further studies that of the physical basis for the interaction between poly(2-ethylacrylic acid) and lipid membranes. The authors suggested that amphiphility, rather than structure, is the most important factor in membrane micellization by macromolecules.

Haensler et al., 1993 describe cascade polymers, also known as Starburst dendrimer polymers, and how when conjugated to plasmids, mediate high efficiency transfection of a variety of suspension and adherent cultured mammalian cells.

Kimoto et al., 1992, described a high molecular weight anticancer agent, polystyrene-co-maleic acid conjugated neocarzinostatin (SMANCS), in which two chains of styrene/maleic acid copolylmer are conjugated to the anticancer protein neocarzinostatin (NCS). The anticancer agent accumulated more selectively in tumor tissue than in normal tissue.

Seymour et al., 1995, describe studies of the in vivo tumour capture of N-(2-hydroxypropyl)methacrylamide (HPMA) co-polymers, using subcutaneous Sarcoma 180 or B16F10 melanoma models. Many soluble macromolecules have the ability to accumulate preferentially in solid tumours. This phenomenon has been named “enhanced permeability and retention” (EPR effect), and results from the increased permeability of tumour vascular endothelium towards circulating macromolecules, combined with limited lymphatic drainage in the tumour interstitium. Increased levels of circulating polymer lead to increased levels in the tumour, and while lower molecular weight polymers (<40 kDa) are subject to rapid renal clearance, higher molecular weight polymers do not escape via the kidney, and persist in circulation for longer.

Thomas et al., 1996, describes the use of fluorescent methods to examine the effect of the pH-dependent adsorption of the hydrophobic polyeletrolyte poly(2-ethylacrylic acid), on the mobility of fluorescent probes in phosphatidylcholine membranes.

Murthy et al., 1999, described attempts to identify polymers which can enhance the transport of endocytosed drugs from the endosomal compartments to the cytoplasm. The pH of an endosome is lower than that of the cytosol by one or two pH units, depending on the stage of endosomal development. Suitable endolytic polymers would disrupt lipid bilayer membranes at pH 6.5 and below, but would be non-lytic at pH 7.4. The authors designed and synthesised pH-sensitive polyacrylate polymers (PPAAc, PEAAc, EA-AAc; see page 138 therein) which hemolyse red blood cells at pH from 6.3 to 5.0, and which have no hemolytic activity at pH 7.4. The authors suggested that pH-sensitive synthetic polymers can be molecularly engineered to efficiently disrupt eukaryotic membranes within defined and narrow pH ranges, and that these polymers might serve as endolytic disruptive agents. The authors acknowledged that the mechanism by which the polymers disrupts lipid bilayers at pHs below 6.0 is not known (see page 140 therein).

Jensen et al., 2001, described studies of the internalization and sub-cellular fate of N-(2-hydroxypropyl)methyacylamide (HPMA) copolymers in Hep G2 (human hepatocellular carcinoma) cells. The polymers are vinyl polymers with a (—CH₂—)ₙ backbone, and are not hypercoiling polymers (see FIG. 2 therein). Galactose was an effective ligand for receptor-mediated endocytosis for Hep G2 cells. Confocal fluorescent microscopy revealed that the polymer entered the cells by endocytosis. After longer incubation times (typically >8 hours), polymer escaped from small vesicles and distributed throughout the cytoplasm and nuclei of the cells. Polymer that entered the cytoplasm tended to accumulate in the nucleus. Microinjection of the HPMA copolymers into cells' cytoplasm and nuclei indicated that the polymer partitioned to the nucleus. Polymers incorporating various fluorescent dyes were also tested and found to have the same properties.

Peptides and Pseudopeptides

Plank et al., 1994, describe endosome-disruptive peptides (EDPs) which mimic naturally-occurring viral fusion peptide sequences, specifically, peptides containing the 20 amino-terminal amino acid sequence of influenza virus hemagglutinin, that apparently are membrane disruptive at pH 5.0 to 6.5, but not at physiological pH.

Ostolaza et al., 1997, described studies of the balance of electrostatic interactions and hydrophobic forces in the process of membrane disruption caused by E. coli α-haemolysin (HlyA), a 107 kDa amphipathic protein. The protein has an isoelectric point at pH 4.1, and, when bound to calcium ions, it binds irreversibly to liposomal membranes, thereby producing vesicle leakage

Weissleder et al., 1999, described a method to image tumor-associated lysosomal protease activity using a biocompatible, optically quenched, near-infrared fluorescence (NIRF) imaging probes which generates a strong NIRF signal after enzyme activation by tumor-associated proteases. Tumoral delivery of the quenched NIRF probe is facilitated using a long, circulating, synthetic graft copolymer. The copolymer is a synthetic graph copolymer consisting of poly-L-lysine (PL) with the ε-amino groups sterically protected by methoxypolyethylene glycol (MPEG) side chains (see FIG. 1(C) therein). This polymer is not a hypercoiling polymer. The copolymer accumulates in tumours by slow leakage across highly permeable neovasculature. Internalization of the copolymer into tumor cells occurs by fluid-phase endocytosis, which is often upregulated in rapidly proliferating cells. Using autoradiography and NIRF microscopy, it was shown that tumoral NIRF signal originated from within the cells with little or no signal from interstitium or necrotic areas.

Marecos et al., 1998, describe similar studies, specifically of the uptake of macromolecular agents in tumor cells (LX-1, human small cell lung carcinoma) and in corresponding xenografts utilizing a long-circulating biocompatible graft copolymer, MPEGs-polylysine-DTPA.

Hypercoiling Polymers

Amphiphilic (also called amphipathic) macromolecules containing charged carboxyl groups together with pendant hydrophobic groups possess the ability to change conformation in response to pH. The conformation of the polymer in aqueous solution depends on a delicate balance between the electrostatic charge repulsion between carboxylate anions and the entropically driven association of hydrophobic groups. Thus, when the pH is sufficiently high, electrostatic repulsion overcomes the hydrophobic attraction, and yields an extended conformation. At lower pH, neutralization of the charged carboxylate anions allows hydrophobic forces to dominate, to give intra-molecular hydrophobic association and collapse of the polymer into a compact conformation, which may result in precipitation of the polymer.

The transition of conformation may be studied, for example, by potentiometric titration. See, for example, Nagasawa et al., 1971.

Mandel et al., 1967, describe studies of the conformational transition, between a compact and an extended state, of poly(methacrylic acid).

Anufrieva et al., 1968, describe studies of the conformational transition, and the compact structures, of polymethacrylic acid (PMA) in aqueous solutions.

Dubin et al., 1970, describe studies of the hydrophobic bonding in alternating copolymers of maleic-acid and alkyl vinyl ethers.

Ohno et al., 1973, describe studies of the pH-induced conformational transition of maleic acid-styrene copolymer in aqueous NaCl solutions using potentiometric titration, viscosimetry, and diolatometry.

Delaire et al., 1984, describe studies of the fluorescence spectroscopy of copolymers of methacrylic acid (MA) and less than 1 mol % of vinyidiphenylanthracene (VDPA). The fluorescence properties of the polymer-bound DPA were shown to change as the pH increased to the point that significant deprotonation of the methacrylic acid occurs.

Yuan et al., 1990, describe studies of the pH-dependent fluorescence and energy transfer properties of water-soluble polyacrylamide polymers labelled with both a donor (eosin) and a receptor (merocyanine).

Anghel et al., 1998, describe studies of the photophysical properties of poly(acrylic acid) labelled with low levels of pyrene, naphthalene, or both chromophores introduced randomly along the chain.

Rutkaite et al., 2001, describe studies of the fluorescent properties, in aqueous solution, of amphiphilic copolymers of carbazole-containing acrylate monomers and methacrylic acid. Fluorescence intensity was found to decrease with increasing carbazole content (due to quenching resulting from hydrophobic association of the carbazole groups), and to be pH dependent.

Eccleston et al., 1999, described the condensation of diamino acids and their derivatives, such as L-lysine, L-lysine ethyl ester, and L-ornithine with aromatic and aliphatic diacyl chlorides, such as iso-phthaloyl chloride. It was suggested that these polymers may show potential as in vivo probes for localized regions of reduced pH often found, for example, in solid tumours (see page 160, therein).

Eccleston et al., 2000, also described the condensation of diamino acids and their derivatives, such as L-lysine and L-lysine ethyl ester with hydrophobic dicarboxylic acid moieties, such as iso-phthaloyl chloride. These polymers were shown to display an ability to associate with the outer membrane of various mammalian cells, which they rupture to varying degrees at pH 5.5. It was shown that Cy3 conjugated poly(L-lysine iso-phthalamide) (no synthesis or structure data information provided) reversibly-adsorbed onto the membrane of CHO cells at pH 5.5. However, further examination by confocal microscopy showed that the adsorption of polymer at pH 5.5 is largely onto the extracellular lipid bilayer, with little internalisation of the polymer apparent (see page 304 and FIG. 6 therein).

### BRIEF DESCRIPTION OF THE DRAWINGS

FIG. 1 shows relative molecular weight distributions for Polydyes 20, 40, 60 and 80 (PD20, PD40, PD60 and PD80) determined from aqueous gel permeation chromatograms in pH 7.0 phosphate buffer.

FIG. 2 shows a fluorescence image of an SDS-PAGE gel, indicating the relative electrophoretic mobilities in free solution of unpolymerised bis-amino Cy3; PD20; PD40; PD60; PD80; a mixture of poly(lysine iso-phthalamide); and bis-amino Cy3.

FIG. 3 shows normalised excitation (□) and emission (◯) spectra of PD20 measured in aqueous solution (0.01 mg/mL) using an Aminco luminescence spectrofluorimeter (λₑₓ 540 nm, λₑₘ 595 nm).

FIG. 4 is a graph showing the relative fluorescence intensity using a Cytofluor plate reader (λₑₓ 535 nm and λₑₘ 570 nm) for solutions of the bis-amino Cy3 (0-0.168 g/l) for a range of concentrations of poly(lysine iso-phthalamide), in PBS at pH 7.4: 0.0 (♦), 0.64 (▴), 0.8 (+), 0.96 (◯), 1.12 (*), 1.28 (▪), 1.44 (□) and 1.6 (⋄) g/l.

FIG. 5 is a graph showing relative fluorescence intensity of PD20 (x), PD40 (Δ), PD60 (□) and PD80 (⋄), and of the bis-amino Cy3 monomer (*), as a function of the concentration of fluorophore containing polymer repeat unit, calculated on the assumption of stoichiometric fluorophore incorporation into the polymer. Absorbance measurements were taken at 553 nm in phosphate buffered saline at pH 7.4.

FIG. 6 is a graph showing relative fluorescence intensity of PD20 (x), PD40 (Δ), PD60 (□) and PD80 (⋄), and of the bis-amino Cy3 monomer (*), as a function of the optical absorbance of each polydye sample (concentration of polymer, 0-2.5 μM). Absorbance measurements were taken at 553 nm in phosphate buffered saline at pH 7.4.

FIG. 7 is a graph showing relative fluorescence intensity of bis-amino Cy3 in aqueous solution (0.26 mM) in the presence of 0.5 g/l poly(lysine iso-phthalamide) as a function of pH.

FIG. 8 is a graph showing relative fluorescence intensity of the bis-sulphonic acid Cy3 derivative in aqueous solution (0.16 mM) in the presence of 0.5 g/l poly(lysine iso-phthalamide) as a function of pH.

FIG. 9 is a graph showing relative fluorescence intensity of aqueous solutions (0.5 g/L) of PD20 (x), PD40 (▴), PD60 (♦), and PD80 (□) titrated with 1.0 N HCl delivered to an Aminco-SDP-125 spectrofluorimeter, equipped with a flow through cell, from a titration vessel via a peristaltic pump. Relative intensities are normalised to PD80 with a fluorophore concentration of 0.58×10⁻⁵ M.

FIG. 10 is a graph showing relative fluorescence intensity of bis-amino Cy3 (0.26 mM) measured in a 0.5 μl aqueous solution of poly(lysine iso-phthalamide) using a Cytofluor plate reader (λₑₓ 535 nm and λₑₘ 570 nm) as a function of sodium chloride (◯) and calcium chloride (◯) concentration.

FIG. 11 is a bar graph showing relative viability as a function of concentration of poly (L-lysine iso-phthalamide) (P3) and poly(L-lysine ethyl ester co-L-lysine iso-phthalamide) (P2), as measured using an MTT cytotoxicity assay with colon-C26 cells.

FIG. 12 is a bar graph showing relative viability as a function of time following acidification of supernatant, in presence of poly(L-lysine dodecanamide) (P1), poly (L-lysine ethyl ester co-L-lysine iso-phthalamide) (P2), or poly(L-lysine iso-phthalamide) (P3), as measured using a modified LDH release assay with COS1 cells.

FIG. 13 is a bar graph showing relative viability as a function of time following acidification of supernatant, in presence of poly(L-lysine dodecanamide) (P1) and poly (L-lysine iso-phthalamide) (P3), as measured using a modified LDH release assay with A2780 cells.

FIG. 14 is a graph of the variation in fluorescence intensity (I) of PD20 at various concentrations (5-100 μg/mL) in the presence (filled symbols) and absence (empty symbols) of serum (10%) at pH 7.4. Fluorescent readings were obtained on a Wallac-Victor² 1420 plate reader (λ_{Ex} 535 nm λ_{Em} 590 nm) at a lamp intensity of 5000.

FIG. 15 is a graph of the variation in the fluorescence intensity of PD20 (♦,⋄), PD40 (▪,□), PD60 (▴,Δ), and PD80 (●,◯) at a concentration of 60 μg/mL as a function of pH (4.0-7.4) in either the presence (filled symbols) or absence (empty symbols) of serum (10%). Fluorescent readings were obtained on a Wallac-Victor² 1420 plate reader (λ_{Ex} 535 nm; λ_{Em} 590 nm) at a lamp intensity of 5000.

FIG. 16 is a graph of the variation in fluorescence intensity (I) of PD20 (60 μg/mL) with varying concentration of serum (0-40%) in buffer (pH 7.4). Fluorescent readings were obtained on a Wallac-Victor² 1420 plate reader (λ_{Ex} 535 nm; λ_{Em} 590 nm) at a lamp intensity of 5000.

FIG. 17 is a graph which compares the fluorescence intensity of hydrophilic SPA beads (A) and hydrophobic SPA beads (B) following incubation with PD20. The fluorescence intensity of hydrophilic SPA beads was relatively low over the pH range 7.4-4.8 whereas the hydrophobic SPA beads fluoresced intensely at pH 5.5 and below due to interaction with the PD20 as the degree of ionisation of the polymer is reduced.

FIG. 18 shows phase contrast images of CHO cells incubated with PD20 at (A) pH 7.4 or (B) pH 5.5, for 30 minutes.

FIG. 19 is a bar graph showing fluorescence intensity calculated from confocal imaging data, as a function of incubation time in the presence of PD20 at pH 7.4, pH 6.0, and pH 5.0.

FIG. 20 shows confocal microscope images recorded for HepG2 cells incubated with a mixture of PD20 and the endosomal stain FITC-Alexa Fluor 488 for 15 minutes at 37° C. Image (A) is for PD20. Image (B) is for FITC-Alexa Fluor 488. Images from A and B were merged to obtain Image (C), indicating co-localisation.

FIG. 21 shows LSCM images recorded for CHO cells 30 minutes after initial incubation at 37° C. at physiological pH (7.4), with (A) bis-sulphonic acid Cy3 (0.01 mg/mL); (B) bis-amino Cy3 (0.01 mg/mL); (C) PD30 (1 mg/mL); and (D) PD30 (1 mg/mL).

FIG. 22 shows LSCM images recorded for HepG2 cells 30 minutes after initial incubation at 37° C. at physiological pH (7.4), with (A) bis-amino Cy3-(0.01 mg/mL); and (B) PD30 (1 mg/mL).

FIG. 23 is a graph of median fluorescence intensity versus exposure time (minutes), as determined using flow cytometry, for 5000 cells, for MCF7 and MCF7/MXR cell lines, for free polymer (poly(L-lysine iso-phthalamide)) with free doxorubicin, without or with DMSO.

FIG. 24 is a graph of median fluorescence intensity versus exposure time (minutes), as determined using flow cytometry, for 5000 cells, for MCF7 and MCF7/MXR cell lines, for poly(L-lysine iso-phthalamide)-conjugated doxorubicin, without or with DMSO.

FIG. 25 is a graph of relative cell viability versus concentration (μM) of (a) doxorubicin, (b) poly(L-lysine iso-phthalamide) conjugated doxorubicin (“PolyDox”), and (c) poly(L-lysine iso-phthalamide) (“Polymer”), as determined using an MTT assay, for MCF7 cells.

FIG. 26 is a graph of relative cell viability versus concentration (μM) of (a) doxorubicin, (b) poly(L-lysine iso-phthalamide) conjugated doxorubicin (“PolyDox”), and (c) poly(L-lysine iso-phthalamide) (“Polymer”), as determined using an MTT assay, for MCF7/MXR cells.

FIG. 27 is a graph of relative cell viability versus concentration (μM) of (a) doxorubicin, (b) poly(L-lysine iso-phthalamide) conjugated doxorubicin (“PolyDox”), and (c) poly(L-lysine iso-phthalamide) (“Polymer”), all in the presence of 1% DMSO, as determined using an MTT assay, for MCF7 cells.

FIG. 28 is a graph of relative cell viability versus concentration (μM) of (a) doxorubicin, (b) poly(L-lysine iso-phthalamide) conjugated doxorubicin (“PolyDox”), and (c) poly(L-lysine iso-phthalamide) (“Polymer”), all in the presence of 1% DMSO, as determined using an MTT assay, for MCF7/MXR cells.

### SUMMARY OF THE INVENTION

One aspect of the present invention pertains to a method delivering a payload into a living cell (e.g., into the nucleus of a living cell), comprising contacting the cell with a hypercoiling carrier polymer which incorporates, or is otherwise associated with, said payload.

Another aspect of the present invention pertains to the use of a hypercoiling carrier polymer for the delivery of a payload into a living cell (e.g., into the nucleus of a living cell), which hypercoiling carrier polymer incorporates, or is otherwise associated with, said payload.

One particularly preferred embodiment is one wherein:

(a) delivery is to the nucleus of the cell (see, e.g., claim 1);

(b) the hypercoiling carrier polymer incorporates said payload (see, e.g., claim 2), for example, the payload forms part of the backbone of the hypercoiling carrier polymer (see, e.g., claim 3) or the payload is tethered to the backbone of the hypercoiling carrier polymer (see, e.g., claim 4);

(c) the carrier polymer does not have a carbon backbone (see, e.g., claim 8), or the carrier polymer is not a vinyl polymer (see, e.g., claim 9), or the carrier polymer has a backbone having amide linkages (see, e.g., claim 10);

(d) the carrier polymer has a molecular weight of from about 1 kDa to about 75 kDa (see, e.g., claim 47);

(e) the carrier polymer and the payload have a combined molecular weight of from about 1 kDa to about 75 kDa (see, e.g., claim 51);

(f) one or more of the hydrophilic moieties bears a carboxylic acid group or a salt thereof (see, e.g., claim 80);

(g) each payload moiety has a gram molecular weight of from about 100 to about 10,000 (see, e.g., claim 114); and,

(h) one or more of the payload moieties are, or comprise, biologically active agents selected from: (a) drugs, prodrugs, chemo-therapeutics, radio-therapeutics, neutron capture agents; and (b) peptides, proteins, antibodies, antibody fragments, enzymes, transcription factors, signalling proteins, antisense peptides, zinc fingers, peptide vaccines (see, e.g., claim 119), or one or more of the payload moieties are, or comprise, detectable labels selected from: (a) fluorophores; (b) chromophores; (c) isotopically enriched species; (d) paramagnetic species; (e) radioactive species; and, (f) scintillents and phosphors (see, e.g., claim 121).

An example of a preferred carrier polymer is a co-polymer of: (a) a monomer selected from iso-phthalic acid and iso-phthaloyl chloride; and, (b) a monomer selected from 2,4-diaminopropionic acid; 2,4-diaminobutyric acid; ornithine; lysine; or 2,6-diaminopimelic acid (see, e.g., claim 108); for example, poly(lysine iso-phthalamide) (see, e.g., claim 109). See also, for example, the “Illustrative Examples” below (page 73).

Another aspect pertains to hypercoiling carrier polymers, as described herein, which are suitable for use in such methods.

Another aspect pertains to hypercoiling carrier polymers, which incorporate a payload, as described herein, which are suitable for use in such methods.

Another aspect pertains to hypercoiling carrier polymers, associated with a payload, as described herein, which are suitable for use in such methods.

Another aspect of the invention pertains to a hypercoiling carrier polymer which incorporates a payload, or which is otherwise associated with a payload, as described herein, for use in a method of treatment of the human or animal body by therapy.

Another aspect of the present invention pertains to use of a hypercoiling carrier polymer which incorporates a payload, or which is otherwise associated with a payload, as described herein, for the preparation of a medicament for the treatment of a condition which is treatable by said payload.

Another aspect of the present invention pertains to a method of treatment of a condition comprising administering to a patient suffering from said condition a therapeutically-effective amount of a hypercoiling carrier polymer which incorporates a payload, or which is otherwise associated with a payload, as described herein, wherein said payload is a drug which treats said condition.

Another aspect of the present invention pertains to a hypercoiling carrier polymer which incorporates a payload, or which is otherwise associated with a payload, as described herein, for use in a method of diagnosis practiced on the human or animal body.

Another aspect of the present invention pertains to a method of diagnosis of a condition comprising:

(a) administering to a patient an effective amount of a hypercoiling carrier polymer which incorporates a payload, or which is otherwise associated with a payload, as described herein, wherein said payload is, or comprises, a detectable label;

(b) detecting the presence and/or location of said detectable label; and

(c) correlating said presence and/or location with said condition.

Another aspect of the present invention pertains to a method of imaging a cell comprising:

(a) contacting a living cell with a hypercoiling carrier polymer which incorporates a payload, or which is otherwise associated with a payload, as described herein, further wherein said payload is, or comprises, a detectable label; and

(b) detecting the presence and/or location of said detectable label.

Another aspect of the present invention pertains to a method of imaging a patient, or a portion thereof, comprising:

(a) administering to said patient an effective amount of a hypercoiling carrier polymer which incorporates a payload, or which is otherwise associated with a payload, as described herein, further wherein said payload is, or comprises, a detectable label; and

(b) detecting the presence and/or location of said detectable label.

As will be appreciated by one of skill in the art, features and preferred embodiments of one aspect of the invention will also pertain to other aspects of the invention.

### DETAILED DESCRIPTION

Early attempts at cellular/nuclear delivery relied upon general circulation in the body. These methods were improved, for example, by relying upon extravasation, that is, the accumulation of high molecular weight species in, for example, tumours. Further improvement was achieved by optimising cell entry mechanisms, for example, endocytosis. Having achieved improved cell entry via endocytosis, efforts then focussed upon endosomal release, in order to dispense the contents into the cell interior, before excessive degradation occurred inside the endosome. One (known) approach for achieving endosomal release relies upon osmotic pressure, which causes the endosome to burst. The inventors have discovered an alternative method for rapid endosomal release, which relies upon the interaction between a hypercoiling polymer and a membrane (e.g., the endosome membrane). Surprisingly and unexpectedly, hypercoiling polymers also facilitate rapid delivery into the nucleus, at a rate far greater than any so far reported.

Thus, one aspect of the present invention pertains to a method delivering a payload into a living cell (e.g., into the nucleus of a living cell), comprising contacting the cell with a hypercoiling carrier polymer which incorporates, or is otherwise associated with, said payload.

“Which Incorporates, or is Otherwise Associated with”

The phrase “which incorporates, or is otherwise associated with” is used herein to encompass numerous alternative embodiments.

The phrase “which incorporates the payload” is intended to pertain to embodiments wherein the payload is covalently bonded to the carrier polymer, for example, by covalent bond(s) and/or linking groups.

In one embodiment, the payload forms part of the backbone of the carrier polymer.

In one embodiment, the payload is tethered to (e.g., conjugated to, pendant from) the backbone of the carrier polymer, either directly or via linking groups.

The phrase “otherwise associated with” (or simply “associated with”) is intended to pertain to embodiments wherein the payload is not covalently bonded to the carrier polymer, but instead forms a complex with the carrier polymer.

In one embodiment, the complex is an ionic complex, that is, the payload and carrier polymer are held together by electrostatic forces (electrostatic attraction), for example, as in a cationic hypercoiling polymer electrostatically complexed to DNA, or as in a hypercoiling anionic polymer electrostatically complexed to a net positively charged DNA polyplex.

In one embodiment, the complex is a coordination complex, that is, the payload and the carrier are held together by coordination bonds (e.g., chelation bonds), for example, as in gadolinium or other MRI contrast agent ion chelated to a porphyrin or diethylenetriamine pentaacetic-acid (DTPA).

In one embodiment, the complex is a lipoplex, that is, the payload and the carrier are held together by lipophilic forces (lipophilic attraction) (also referred to as hydrophobic forces and hydrophobic attraction), for example, as in a hypercoiling polymer anchored via a lipophilic chain to a liposome, or as in a hypercoiling polymer hypercoiled around a hydrophobic payload.

In one embodiment, the complex is a ligand-receptor complex, that is, the payload and the carrier are held together by ligand-receptor interactions (e.g., biotin-streptavidin interactions), for example, as in hypercoiling polymer bearing biotin or streptavidin bound to a payload bearing streptavidin or biotin, respectively.

Carrier Polymer: Hypercoiling

The carrier polymer is a hypercoiling polymer, that is, a hypercoiling carrier polymer. More particularly, the carrier polymer which incorporates the payload, or the carrier polymer when otherwise associated with the payload, is hypercoiling.

Note that while all hypercoiling polymers are amphiphilic (see below), not all amphiphilic polymers are hypercoiling.

The term “hypercoiling,” as used herein, pertains to a polymer which undergoes a hypercoiling transition upon change of pH, for example across a threshold pH.

The term “hypercoiling transition,” as used herein, pertains to the transition of a polymer from a rod to a globule (e.g., coil, helix), or from a globule (e.g., coil, helix) to a rod.

In this respect, the carrier polymer is a “pH responsive polymer,” that is, the polymer changes conformation in response to a change in pH, for example across a threshold pH.

In one embodiment, the hypercoiling transition occurs within (and the threshold pH falls within) a pH range of 2 to 12; a pH range of 4 to 9; a pH range of 5 to 7.5.

In one embodiment, the carrier polymer undergoes a hypercoiling transition, from a rod to a globule (e.g., coil, helix), as the pH is increased across the threshold pH. More specifically, the polymer has a rod conformation below the threshold pH, and a globule (e.g., coil, helix) conformation above the threshold pH.

In one embodiment, the carrier polymer undergoes a hypercoiling transition, from a globule (e.g., coil, helix) to a rod, as the pH is increased across the threshold pH. More specifically, the polymer has a globule (e.g., coil, helix) conformation below the threshold pH, and a rod conformation above the threshold pH.

For example, in one embodiment, where changes in pH determines the degree of ionization, the carrier polymer changes from an extended (e.g., rod) conformation at high degrees of ionization to a compact conformation stabilized by hydrophobic association at low degrees of ionization. In some cases, at low or very low degrees of ionization, the carrier polymer may precipitate from solution.

Methods for determining whether or not a polymer is in fact a hypercoiling polymer are well known in the art. Such methods permit, for example, the detection of a hypercoiling transition, and a determination of the threshold pH for the transition. Examples of some representative methods are described herein.

Identifying Hypercoiling Polymers: by Light Scattering

One way to identify a candidate polymer as a hypercoiling polymer is by a significant change in radius of gyration upon change in pH. The radius of gyration may be determined, for example, by using light scattering methods. See, for example, Atkins, 1989.

For example, in a typical study, the radius of gyration of the candidate polymer is measured using a light scattering instrument over a range of concentrations (dependant on the specific polymer) and over a range of pH. A sudden change in radius of gyration over a narrow pH range (e.g., 0.1-0.5 pH units) is evidence of a hypercoiling polymer.

For example, in a solution with a pH substantially higher than the pKa of weak Bronsted acid groups of a hypercoiling polymer, the weak Bronsted acid groups are deprotonated and anionic, and the resulting charge repulsion between the anionic groups forces the polymer to adopt an extended rod-like conformation.

As the pH of the solution is reduced and approaches the pKₐ of the weak acid groups, the degree of ionisation is reduced. At this point, the hydrophobic groups coalesce and pull the polymer into a tightly coiled or globule (hypercoiled) conformation, resulting in a sharp decrease in the hydrodynamic volume. In contrast, in the absence of hydrophobic association the polymer conformation changes from the extended state to a statistical coil determined by the Brownian motion of the segments of the polymer. A lack of hydrophobic stabilisation causes the transition to be more gradual and the random coil has a larger radius of gyration.

For example, a polymer is considered to be a hypercoiling polymer if it exhibits a change in radius of gyration of at least about 20% (e.g., a factor of less than 0.8) (e.g., from 1.0 to 0.8 units) over a small pH range of no more than about 1.0 pH units breadth, which small range falls within a large range of about pH 4 to about pH 9.

Preferably, the change in radius of gyration is at least about 30% (e.g., a factor of less than 0.7) (e.g., from 1.0 to 0.7 units);

at least about 40% (e.g., a factor of less than 0.6) (e.g., from 1.0 to 0.6 units);

at least about 50% (e.g., a factor of less than 0.5) (e.g., from 1.0 to 0.5 units);

at least about 60% (e.g., a factor of less than 0.4) (e.g., from 1.0 to 0.4 units);

at least about 70% (e.g., a factor of less than 0.3) (e.g., from 1.0 to 0.3 units);

at least about 80% (e.g., a factor of less than 0.8) (e.g., from 1.0 to 0.2 units).

Preferably, the small pH range is no more than about 0.7 pH units; no more than about 0.5 pH units; no more than about 0.3 pH units; no more than about 0.1 pH units.

Identifying Hypercoiling Polymers: by Potentiometric Titration

One way to identify a candidate polymer as a hypercoiling polymer is by potentiometric titration methods. See, for example, Leyte et al., 1964.

For example, in a typical study, a 25 mL sample of a 0.01 N solution an anionic polymer is titrated with dilute hydrochloric acid (typically 0.1 M) under a nitrogen atmosphere at constant temperature (25° C.). The degree of ionization is plotted against the apparent PKₐ.

For example, a hypercoiling polymer is characterized by a rapid increase in apparent pKₐ at low degrees of ionization followed by a decrease in apparent pKₐ at intermediate degrees of ionization. Non-hypercoiling polymers exhibit a progressive increase in apparent pKₐ over the whole range of ionization.

For example, during the potentiometric titration of an anionic hypercoiling polymer, the polymer is held in a tight coil due to the association of the hydrophobic groups. Therefore, initially the size of the polymer changes slowly, resulting in a increasing charge density within the coil, with a consequent rapid increase in the apparent pKₐ of the titratable groups. Once the polymer has expanded sufficiently to overcome the short range hydrophobic interactions, the polymer dimensions increase considerably, resulting in a reduction in the charge density and the apparent pKₐ. The remaining groups titrate normally.

In one embodiment, the rapid increase in apparent pKₐ occurs within a degree of ionisation in the range of about 0.1 to about 0.9.

In one embodiment, the range is about 0.1 to about 0.8; about 0.2 to about 0.7; about 0.2 to about 0.6; about 0.2 to about 0.5; about 0.2 to about 0.4.

Identifying Hypercoiling Polymers: by Viscometry

One way to identify a candidate polymer as a hypercoiling polymer is by a significant change in radius of gyration upon change in pH. The radius of gyration may be determined, for example, by using viscometry methods. See, for example, Atkins, 1989.

For example, in a typical study, the specific viscosity of a solution of the candidate polymer is measured using, for example, an Ubbelholde viscometer, for a range of concentrations (dependant on the specific polymer) and a range of pH.

For example, in the case of a hypercoiling polymer, the intrinsic viscosity is almost constant at low degrees of ionisation, but increases rapidly at intermediate degrees of ionisation, and levels off or decreases slightly as the degree of ionisation increases further. In the case on a non-hypercoiling polymer the intrinsic viscosity increases progressively over the whole range of ionisation. This effect for hypercoiling polymers is due to the hydrophobic stabilisation of the coil structure at low degrees of ionisation. Only when the electrostatic repulsion is sufficient to over come the association of the hydrophobic groups at some critical degree of ionisation does the coil increase in size with a concomitant increase in viscosity. See, for example, Leyte et al., 1964.

In one embodiment, the rapid increase in viscosity occurs within a degree of ionisation in the range of about 0.1 to about 0.9.

In one embodiment, the range is about 0.1 to about 0.8. about 0.1 to about 0.7; about 0.1 to about 0.6; about 0.1 to about 0.5; about 0.2 to about 0.6.

Carrier Polymer: Architecture

In general, hypercoiling polymers have a relatively flexible backbone; which permits a hypercoiling transition. For example, some polymers have a relatively rigid backbone, and do not undergo a hypercoiling transition. In contrast, polymers such as those described herein have a relatively flexible backbone, and do undergo a hypercoiling transition. Thus, a flexible backbone is necessary, but not sufficient, to permit a hypercoiling transition.

As discussed in more detail below, the carrier polymer is amphiphilic (amphipathic) and has hydrophobic regions and hydrophilic regions. (Note that all hypercoiling polymers are amphiphilic polymers, but that not all amphiphilic polymers are hypercoiling polymers.) The hydrophobic regions derive their hydrophobic character from hydrophobic moieties. The hydrophilic regions derive their hydrophilic character from hydrophilic moieties. Some or all of these hydrophobic moieties and hydrophilic moieties participate in the hypercoiling transition.

In addition to these hydrophobic regions and hydrophilic regions, the carrier polymer may have other regions, for example, which are neither strongly hydrophobic nor hydrophilic, and which do not substantially contribute to the hypercoiling transition, but which do not prevent the hypercoiling transition. This allows some variation in the composition and architecture of the polymer.

For example, in those embodiments where the carrier polymer incorporates the payload, the payload may form a region of the carrier polymer which is neither strongly hydrophobic nor hydrophilic, and which does not substantially contribute to (but does not prevent) the hypercoiling transition; alternatively, the payload may also function as a hydrophilic or hydrophobic moiety or region.

The balance between the hydrophobic regions and the hydrophilic regions determines whether or not the polymer is a hypercoiling polymer, and also determines the pH at which the hypercoiling transition occurs. This balance is a function of the nature (e.g., intrinsic hydrophobicity/hydrophilicity), number (e.g., mole fraction, weight fraction), and relative positioning (e.g., alternating, random, with or without spacers, etc.) of the hydrophobic and hydrophilic regions.

Carrier Polymer: Biocompatible, Non-Cytotoxic

In one embodiment, the carrier polymer is biocompatible.

The term “biocompatible”, as used herein, pertains to a polymer which is substantially non-toxic, that is, substantially non-cytotoxic, e.g., towards the cells with which it is to be used.

Methods (e.g., MTT assays and LDH release assays) for determining whether or not a particular carrier polymer is biocompatible (non-cytotoxic) are described in detail below.

For example, a carrier polymer is considered to be substantially biocompatible (non-cytotoxic) if it has a relative viability, using an MTT assay, e.g., as described below, of above 50%, at a carrier polymer concentration of 350 μg/mL; and/or a relative viability, using an LDH release assay, e.g., as described below, of above 50%, at a carrier polymer concentration of 350 μg/mL.

Carrier Polymer: Biodegradable

In one embodiment, the carrier polymer is biodegradable.

The term “biodegradable”, as used herein, pertains to a polymer which is substantially degraded in vivo, e.g., in (or in contact with) the cells with which it is to be used.

Methods (e.g., degradation assays) for determining whether or not a particular carrier polymer is biodegradable are well known.

For example, in one type of assay, polymer in solution (e.g., about 1-50 mg/mL) is incubated both with and without enzymes (e.g., a protease), for an appropriate time (e.g., hours, days, weeks), at a suitable pH (e.g., 7.4, 6.5, 6.0, 5.5), and the molecular weight monitored over time, for example, using gel permeation chromatography (GPC). A reduction in molecular weight indicates the polymer is biodegradable.

In one embodiment, the reduction is at least 5% (e.g., from 100 to 95 kDa);

at least 10% (e.g., from 100 to 90 kDa); at least 20% (e.g., from 100 to 80 kDa);

at least 30% (e.g., from 100 to 70 kDa); at least 50% (e.g., from 100 to 50 kDa).

Without wishing to be bound to any particular theory, it is postulated that degradation to a molecular weight which is below the renal threshold (typically about 30 kDa for a globular polymer) is desired.

Carrier Polymer: Backbone

In one embodiment, the carrier polymer does not have a carbon backbone.

Examples of polymers which have a carbon backbone include, but are not limited to, vinyl polymers and phenol-formaldehyde polymers.

In one embodiment, the carrier polymer is not a vinyl polymer.

Examples of vinyl polymers include, e.g., poly(vinyl chloride)s, poly(ethylene)s, poly(propylene)s, poly(styrene)s, poly(acrylic acid)s, poly(acrylate)s, poly(acrylamide)s, poly(acrylonitrile)s, and co-polymers thereof.

Examples of polymers which are not vinyl polymers include, but are not limited to, polyamides, polyesters, polyethers, polysulfides, and polysulfones.

In one embodiment, the carrier polymer does not have monomer units derived from a vinyl compound.

Examples of vinyl compounds include, but are not limited to, vinyl chloride, ethylene, propylene, styrene, acrylic acid, acrylate, acrylamide, and acrylonitrile.

Carrier Polymer: Backbone: Amide Groups

In one embodiment, the carrier polymer has a backbone having amide linkages. In one embodiment, the backbone also has other linkages.

An amide linkage is —C(═O)—NR—. The group R may be a monodentate group, or it may be a polydentate (e.g., bidentate) group which forms part of a cyclic group (e.g., as in proline).

In one embodiment, the carrier polymer is a polyamide, that is, a polymer having amide linkages. In one embodiment, the carrier polymer also has other linkages.

In one embodiment, the carrier polymer has a backbone having peptide linkages. In one embodiment, the backbone also has amide linkages. In one embodiment, the backbone also has other linkages.

A peptide linkage is an amide linkage which is formed by reaction of a α-amino group of one α-amino acid and an α-carboxylic acid group of another α-amino acid.

In one embodiment, the carrier polymer is a polypeptide (protein), that is a polymer having peptide linkages. In one embodiment, the carrier polymer also has amide linkages. In one embodiment, the carrier polymer also has other linkages.

In one embodiment, the carrier polymer has a backbone having pseudo-peptide linkages. In one embodiment, the backbone also has peptide linkages. In one embodiment, the backbone also has peptide and/or amide linkages. In one embodiment, the backbone also has other linkages.

A pseudo-peptide linkage is an amide linkage which is not formed by reaction of a α-amino group of one α-amino acid and an α-carboxylic acid group of another α-amino acid.

Pseudo-peptide linkages may be formed, for example, by reaction of side chain amino groups reacting with α-carboxylic acid groups; sidechain-carboxylic acid groups reacting with α-amino groups; side chain amino groups reacting with side chain carboxylic acid groups; etc. The resulting pseudo-peptide linkages may, or may not, give rise to a polymer branch point.

In one embodiment, the carrier polymer is a pseudo-polypeptide (pseudo-protein), that is, a polymer having pseudo-peptide linkages. In one embodiment, the carrier polymer also has peptide linkages. In one embodiment, the carrier polymer also has peptide and/or amide linkages. In one embodiment, the carrier polymer also has other linkages.

Without wishing to be bound to any particular theory, it is believed that by employing such amide bonds, the resulting carrier polymer has improved biocompatibility and/or biodegradability.

Carrier Polymer: Backbone: Ester Groups

In one embodiment, the carrier polymer has a backbone having ester linkages. In one embodiment, the backbone also has other linkages.

An ester linkage is —C(═O)—O—.

In one embodiment, the carrier polymer is a polyester, that is, a polymer having ester linkages. In one embodiment, the carrier polymer also has other linkages.

In one embodiment, the carrier polymer has a backbone having ester linkages and amide linkages. In one embodiment, the backbone also has other linkages.

In one embodiment, the carrier polymer is a poly ester amide, that is, a polymer having ester linkages and amide linkages. In one embodiment, the carrier polymer also has other linkages.

For example, a polymer formed by polymerisation of tyrosine or hydroxytryptophan, has ester linkages (e.g., by reaction of the phenolic hydroxy group with the carboxylic acid group) and amide linkages (by reaction of the alpha amino group with the carboxylic acid group).

Carrier Polymer: Metabolic Components

In one embodiment, the carrier polymer comprises a substantial fraction of metabolic components (e.g., monomer units), for example, components derived from amino acids, acids from the Krebs cycle, and components of food additives.

In one embodiment, the carrier polymer comprises a substantial fraction of components (e.g., monomer units) derived from one or more of the following: lysine; hydroxylysine; ornithine; diaminopimelic acid; diaminobutyric acid; diaminopropionic acid; diaminosuccinic acid; serine; 5-hydroxytryptophan; hyroxyhistidine; m-tyrosine; p-tyrosine; o-tyrosine; cysteine; cystine; citric acid; citraconic acid; aconitic acid; isocitric acid; oxalosuccinic acid; ketoglutaric acid; succinic acid; fumaric acid; malic acid; oxalolactic acid; glutaric acid; glutamic acid; aspartic acid; malonic acid, and alkyl and aryl derivatives thereof; phenylphosphonic diacid; alanine; valine; leucine; phenylalanine; phenylglycine; glycine; tryptophan; histidine; isovaline; isoleucine; norvaline; norleucine; adipic acid.

In one embodiment, the carrier polymer comprises at least about 50% by weight of such components.

In one embodiment, the amount is at least about 60%; at least about 70%;

at least about 80%; at least about 90%.

In one embodiment, the carrier polymer and the payload (that is, the carrier polymer which incorporates the payload, or the carrier polymer and the otherwise associated payload) comprises at least about 50% by weight of such components.

In one embodiment, the amount is at least about 60%; at least about 70%; at least about 80%; at least about 90%.

Such components may form part of the polymer backbone and/or polymer side chains.

For example, such components may be used to form aryl esters (e.g., by reaction of a pendant carboxylic acid group with the phenolic hydroxyl group of tyrosine); to form thioamides (e.g., by reaction of a pendant amino group with the thiol group of cysteine), to form esters (e.g., by reaction of a pendant carboxylic acid group with the hydroxyl group of serine), etc.

Without wishing to be bound to any particular theory, it is believed that by employing such components, the resulting carrier polymer has improved biocompatibility and/or biodegradability.

In one embodiment, the carrier polymer is synthetic, that is, it is not naturally occurring, for example, it is not a naturally occurring peptide or protein.

Carrier Polymer: Amphiphilic

The carrier polymer is amphiphilic (also referred to as amphipathic).

Note that all hypercoiling polymers are amphiphilic. But note that not all amphiphilic polymers are hypercoiling polymers.

More particularly, the carrier polymer which incorporates the payload, or the carrier polymer when otherwise associated with the payload, is amphiphilic.

The term “amphiphilic” (and “amphipathic”), as used herein, pertains to a polymer which has both hydrophobic regions and hydrophilic regions.

Some or all of these hydrophobic regions and hydrophilic regions participate in the hypercoiling transition of the carrier polymer.

Carrier Polymer: Hydrophobic Regions

The carrier polymer has hydrophobic regions. These hydrophobic regions derive their hydrophobic character from hydrophobic moieties. Separate hydrophobic regions may be identical or different. Each hydrophobic region may comprise exactly one hydrophobic moiety; one or more hydrophobic moieties, which may be identical or different; or more than one hydrophobic moieties, which may be identical or different.

Hydrophobic moieties/regions may be introduced by, or incorporated by, or result from, the use of a monomer providing a hydrophobic moiety, or a monomer which has been modified to provide a hydrophobic moiety.

Carrier Polymer: Hydrophilic Regions

The carrier polymer has hydrophilic regions. These hydrophobic regions derive their hydrophilic character from hydrophilic moieties. Separate hydrophilic regions may be identical or different. Each hydrophilic region may comprise exactly one hydrophilic moiety; one or more hydrophilic moieties, which may be identical or different; or more than one hydrophilic moieties, which may be identical or different.

Hydrophilic moieties/regions may be introduced by, or incorporated by, or result from, the use of a monomer providing a hydrophilic moiety.

As discussed below, unless otherwise specified, a reference to a hydrophilic region or moiety is intended to be a reference to a “pH responsive” hydrophilic moiety or region, that is, a hydrophilic moiety or region which contributes to the pH responsive nature of the carrier polymer of which it forms a part (see below).

Carrier Polymer: Hydrophilic Regions and Moieties: Relative Positioning

In one embodiment, the hydrophobic regions and hydrophilic regions alternate along the length of the backbone of the carrier polymer.

In one embodiment, the hydrophobic moieties and hydrophilic moieties alternate along the length of the backbone of the carrier polymer.

Carrier Polymer: Hydrophilic Regions and Moieties: Numbers

In one embodiment, the carrier polymer has from 10 to 500 hydrophilic regions; from 50 to 400 hydrophilic regions; from 100 to 300 hydrophilic regions.

In one embodiment, the carrier polymer has from 10 to 500 hydrophilic moieties; from 50 to 400 hydrophilic moieties; from 100 to 300 hydrophilic moieties.

Carrier Polymer: Hydrophobic Regions and Moieties: Numbers

In one embodiment, the carrier polymer has from 10 to 500 hydrophobic regions; from 50 to 400 hydrophobic regions; from 100 to 300 hydrophobic regions.

In one embodiment, the carrier polymer has from 10 to 500 hydrophobic moieties; from 50 to 400 hydrophobic moieties; from 100 to 300 hydrophobic moieties.

Carrier Polymer: Hydrophobic and Hydrophilic Regions: Number Ratios

In one embodiment, the ratio of hydrophilic regions to hydrophobic regions, by number, for the carrier polymer is from about 0.2 (1:5) to about 5 (5:1).

In one embodiment, the ratio is from about 0.4 (2:5) to about 2.5 (5:2).

In one embodiment, the ratio is from about 0.4 (2:5) to about 0.6 (3:5).

In one embodiment, the ratio is about 0.5 (1:2).

In one embodiment, the ratio is from about 0.8 (8:10) to about 1.2 (12:10).

In one embodiment, the ratio is about 1 (1:1).

In one embodiment, the ratio is from about 1.8 (18:10) to about 2.2 (22:10).

In one embodiment, the ratio is about 2 (2:1).

Carrier Polymer: Hydrophobic and Hydrophilic Moieties: Number Ratios

In one embodiment, the ratio of hydrophilic moieties to hydrophobic moieties, by number, for the carrier polymer is from about 0.2 (1:5) to about 5 (5:1).

In one embodiment, the ratio is from about 0.4 (2:5) to about 2.5 (5:2).

In one embodiment, the ratio is from about 0.4 (2:5) to about 0.6 (3:5).

In one embodiment, the ratio is about 0.5 (1:2).

In one embodiment, the ratio is from about 0.8 (8:10) to about 1.2 (12:10).

In one embodiment, the ratio is about 1 (1:1).

In one embodiment, the ratio is from about 1.8 (18:10) to about 2.2 (22:10).

In one embodiment, the ratio is about 2 (2:1).

Carrier Polymer: Hydrophobic Regions: Content by Weight

In one embodiment, the hydrophobic regions account for about 1-90% by weight of the carrier polymer. In one embodiment, the amount is about 5-70%; about 10-60%; about 20-50%; about 20-40%; about 20-30%.

In one embodiment, the hydrophobic regions account for about 0.01-90% by weight of the carrier polymer and the payload (that is, the carrier polymer which incorporates the payload, or the carrier polymer and the otherwise associated payload).

In embodiments where the total payload is relatively large (e.g., more than about 10⁶ Da), the amount is about 0.01-5%. In one embodiment, the amount is about 0.05-5%; about 0.1-5%.

In embodiments where the total payload is not relatively large (e.g., less than about 10⁶ Da), the amount is about 1-90%. In one embodiment, the amount is about 5-70%. about 10-60%; about 20-50%; about 20-40%; about 20-30%.

Carrier Polymer Hydrophobic Moieties: Content by Weight

In one embodiment, the hydrophobic moieties account for about 1-90% by weight of the carrier polymer. In one embodiment, the amount is about 5-70%; about 10-60%; about 20-50%; about 20-40%; about 20-30%.

In one embodiment, the hydrophobic moieties account for about 0.01-90% by weight of the carrier polymer and the payload (that is, the carrier polymer which incorporates the payload, or the carrier polymer and the otherwise associated payload).

In embodiments where the total payload is relatively large (e.g., more than about 10⁶ Da), the amount is about 0.01-5%; about 0.05-5%; about 0.1-5%.

In embodiments where the total payload is not relatively large (e.g., less than about 10⁶ Da), the amount is about 1-90%; about 5-70%; about 10-60%; about 20-50%; about 20-40%; about 20-30%.

For example, a poly(lysine-iso-phthalamide) polymer, having alternating lysine and iso-phthalic acid units (each pair has MW 276), has about 41% by weight carboxylic acid-substituted pentylene groups (derived from lysine, —(CH₂)₄CH(COOH)—, MW 114) and about 27% by weight phenylene groups (derived from iso-phthalic acid, —C₆H₄—, MW 76).

Carrier Polymer: Hydrophilic Regions: Content by Weight

In one embodiment, the hydrophilic regions account for about 1-90% by weight of the carrier polymer. In one embodiment, the amount is about 5-70%; about 10-60%; about 20-50%; about 20-40%; about 20-30%.

In one embodiment, the hydrophilic regions account for about 0.01-90% by weight of the carrier polymer and the payload (that is, the carrier polymer which incorporates the payload, or the carrier polymer and the otherwise associated payload).

In embodiments where the total payload is relatively large (e.g., more than about 10⁶ Da), the amount is about 0.01-5%; about 0.05-5%; about 0.1-5%.

In embodiments where the total payload is not relatively large (e.g., less than about 10⁶ Da), the amount is about 1-90%. In one embodiment, the amount is about 5-70%; about 10-60%; about 20-50%; about 20-40%; about 20-30%.

Carrier Polymer: Hydrophilic Moieties: Content by Weight

In one embodiment, the hydrophilic moieties account for about 1-90% by weight of the carrier polymer. In one embodiment, the amount is about 5-70%; about 10-60%; about 20-50%; about 20-40%; about 20-30%.

In one embodiment, the hydrophilic moieties account for about 0.01-90% by weight of the carrier polymer and the payload (that is, the carrier polymer which incorporates the payload, or the carrier polymer and the otherwise associated payload).

In embodiments where the total payload is relatively large (e.g., more than about 10⁶ Da), the amount is about 0.01-5%; about 0.05-5%; about 0.1-5%.

In embodiments where the total payload is not relatively large (e.g., less than about 10⁶ Da), the amount is about 1-90%. In, one embodiment, the amount is about 5-70%; about 10-60%; about 20-50%; about 20-40%; about 20-30%.

Carrier Polymer: Hydrophobic and Hydrophilic Regions and Moieties: Weight Ratios

In one embodiment, the ratio of (all of the) hydrophilic regions to (all of the) hydrophobic regions, by weight, for the carrier polymer is from about 0.1 to about 5. In one embodiment, the ratio is from about 0.1 to about 4; from about 0.2 to about 3; from about 0.4 to about 2.5; from about 0.5 to about 2.

In one embodiment, the ratio of (all of the) hydrophilic regions to (all of the) hydrophobic regions, by weight, for the carrier polymer and the payload (that is, the carrier polymer which incorporates the payload, or the carrier polymer and the otherwise associated payload) is from about 0.1 to about 5. In one embodiment, the ratio is from about 0.1 to about 4; from about 0.2 to about 3; from about 0.4 to about 2.5; from about 0.5 to about 2.

Carrier Polymer: Hydrophobic Regions and Moieties: Molecular Weight

In one embodiment, each hydrophobic region has a gram molecular weight of from about 14 to about 1000.

In one embodiment, each hydrophobic moiety has a gram molecular weight of from about 14 to about 1000.

In one embodiment, the range is from about 14 to about 700; from about 14 to about 500; from about 14 to about 300; from about 14 to about 200.

In one embodiment, the range is from about 28 to about 1000; from about 28 to about 700; from about 28 to about 500; from about 28 to about 300; from about 28 to about 200.

In one embodiment, the range is from about 76 to about 1000; from about 76 to about 700; from about 76 to about 500; from about 76 to about 300; from about 76 to about 200.

For example, a methylene group, —CH₂—, has a gram molecular weight of 14;

an ethylene group, —CH₂CH₂—, has a gram molecular weight of 28; and,

a phenylene group, —C₆H₄—, has a gram molecular weight of 76.

Note that the iso-phthalic acid repeating unit in poly(lysine iso-phthalamide), —C(═O)—C₆H₄—C(═O)—, has a gram molecular weight of 132.

Carrier Polymer: Hydrophilic Regions and Moieties: Molecular Weight

In one embodiment, each hydrophilic region has a gram molecular weight of from about 16 to about 1000.

In one embodiment, each hydrophilic moiety has a gram molecular weight of from about 16 to about 1000.

In one embodiment, the range is from about 16 to about 700; from about 16 to about 500; from about 16 to about 300; from about 16 to about 200.

In one embodiment, the range is from about 44 to about 1000; from about 44 to about 700; from about 44 to about 500; from about 44 to about 300; from about 44 to about 200.

In one embodiment, the range is from about 57 to about 1000; from about 57 to about 700; from about 57 to about 500; from about 57 to about 300; from about 57 to about 200.

For example, an amino group, —NH₂, has a gram molecular weight of 16;

a dimethylamino group, —NMe₂, has a gram molecular weight of 44;

a carboxylate group, —COO⁻, has a gram molecular weight of 44; and,

a methylene carboxylate group, —CH(COO⁻)—, has a gram molecular weight of 57.

Note that the lysine repeating unit in poly(lysine iso-phthalamide), —NH—(CH₂)₄—CH(COOH)NH—, has a gram molecular weight of 144.

Carrier Polymer: Molecular Weight

The carrier polymer has a molecular weight of less than about 1 MDa; less than about 500 kDa; less than about 200 kDa; less than about 100 kDa; less than about 75 kDa; less than about 50 kDa; less than about 25 kDa.

In one embodiment, the carrier polymer has a molecular weight of from about 1 kDa to about 1 MDa.

In one embodiment, the range is from about 1 kDa to about 500 kDa; from about 1 kDa to about 200 kDa; from about 1 kDa to about 100 kDa; from about 1 kDa to about 75 kDa; from about 1 kDa to about 50 kDa; from about 1 kDa to about 25 kDa.

In one embodiment, the range is from about 10 kDa to about 1 MDa; from about 10 kDa to about 500 kDa; from about 10 kDa to about 200 kDa; from about 10 kDa to about 100 kDa; from about 10 kDa to about 75 kDa; from about 10 kDa to about 50 kDa; from about 10 kDa to about 25 kDa.

In one embodiment, the range is from about 100 kDa to about 1 MDa; from about 100 kDa to about 500 kDa; from about 100 kDa to about 200 kDa; from about 75 kDa to about 100 kDa; from about 50 kDa to about 100 kDa; from about 50 kDa to about 75 kDa; from about 25 kDa to about 75 kDa; from about 25 kDa to about 50 kDa.

In one embodiment, the carrier polymer and the payload (that is, the carrier polymer which incorporates the payload, or the carrier polymer and the otherwise associated payload) have a combined molecular weight of less than about 1 MDa.

In one embodiment, the weight is less than about 500 kDa; less than about 200 kDa; less than about 100 kDa; less than about 75 kDa; less than about 50 kDa; less than about 25 kDa.

In one embodiment, the carrier polymer and the payload have a combined molecular weight of from about 1 kDa to about 1 MDa.

In one embodiment, the range is from about 1 kDa to about 500 kDa; from about 1 kDa to about 200 kDa; from about 1 kDa to about 100 kDa; from about 1 kDa to about 75 kDa; from about 1 kDa to about 50 kDa; from about 1 kDa to about 25 kDa.

In one embodiment, the range is from about 10 kDa to about 1 MDa; from about 10 kDa to about 500 kDa; from about 10 kDa to about 200 kDa; from about 10 kDa to about 100 kDa; from about 10 kDa to about 75 kDa; from about 10 kDa to about 50 kDa; from about 10 kDa to about 25 kDa.

In one embodiment, the range is from about 100 kDa to about 1 MDa; from about 100 kDa to about 500 kDa; from about 100 kDa to about 200 kDa; from about 75 kDa to about 100 kDa; from about 50 kDa to about 100 kDa; from about 50 kDa to about 75 kDa; from about 25 kDa to about 75 kDa; from about 25 kDa to about 50 kDa.

Without wishing to be bound to any particular theory, it is believed that the upper bound is determined by translocation through or across nuclear membrane.

Note that modification of the polymer to include other components (e.g., PEG, etc.; see below) may substantially increase the molecular weight, e.g., towards the high end of the ranges recited above.

Carrier Polymer: Some Preferred Hydrophobic Moieties

In one embodiment, exactly one, one or more, more than one, or all, of the hydrophobic moieties are selected from moieties derived from:

(a) an alkane, having from 1 to 20 carbon atoms;

(b) an alkene or an alkyne having from 2 to 20 carbon atoms;

(c) a cycloalkane, a cylcoalkene, or a cycloalkyne, having from 3 to 20 carbon atoms;

(d) a carboarene having from 6 to 20 ring carbon atoms;

(e) a heteroarene having from 5 to 20 ring atoms;

(f) a heterocycle having from 5 to 20 ring atoms;

(g) an alkane, an alkene, an alkyne, a cycloalkane, a cylcoalkene, or a cycloalkyne, as defined above, attached to a carboarene as defined above;

(h) an alkane, an alkene, an alkyne, a cycloalkane, a cylcoalkene, or a cycloalkyne, as defined above, attached to a heteroarene as defined above; and;

(i) an alkane, an alkene, an alkyne, a cycloalkane, a cylcoalkene, or a cycloalkyne, as defined above, attached to a heterocycle as defined above.

Note that the term “derived from,” as used herein, indicates that the specified group may be derived from the specified source, and not that it is necessarily derived from the specified source.

Note that the term “all,” as used herein; indicated-substantially all, for example, more than 95%, preferably more than 99%, by number.

In one embodiment, the selection is from moieties derived from: (a) and (b).

In one embodiment, the selection is from moieties derived from: (d) and (e).

In one embodiment, the selection is from moieties derived from: (g), (h), and (i).

In one embodiment, the selection is from moieties derived from: (c), (d), (e), (f), (g), (h), and (i).

In one embodiment, the selection is from moieties derived from: (c), (d), (e), and (f).

In one embodiment, the selection is from moieties derived from: (d) and (g).

In one embodiment, the selection is from moieties derived from: (e) and (h).

In one embodiment, the selection is from moieties derived from: (f) and (i).

In one embodiment, the selection is from moieties derived from: (a).

In one embodiment, the selection is from moieties derived from: (b).

In one embodiment, the selection is from moieties derived from: (c).

In one embodiment, the selection is from moieties derived from: (d).

In one embodiment, the selection is from moieties derived from: (e).

In one embodiment, the selection is from moieties derived from: (f).

In one embodiment, the selection is from moieties derived from: (g).

In one embodiment, the selection is from moieties derived from: (h).

In one embodiment, the selection is from moieties derived from: (i).

In one embodiment, exactly one of the hydrophobic moieties is so selected.

In one embodiment, one or more of the hydrophobic moieties is so selected.

In one embodiment, more than one of the hydrophobic moieties are so selected.

In one embodiment, all of the hydrophobic moieties are so selected.

Examples of alkanes include, but are not limited to, methane (C₁), ethane (C₂), propane (C₃), butane (C₄), pentane (C₅), hexane (C₆), heptane (C₇), octane (C₈), nonane (C₉), decane (C₁₀), undecane (C₁₁), dodecane (C₁₂), tridecane (C₁₃), tetradecane (C₁₄), pentadecane (C₁₅), eicodecane (C₂₀).

Examples of linear alkanes include, but are not limited to, methane (C₁), ethane (C₂), n-propane (C₃), n-butane (C₄), n-pentane (amane) (C₅), n-hexane (C₆) n-heptane (C₇).

Examples of branches alkanes include, but are not limited to, iso-propane (C₃), iso-butane (C₄), sec-butane (C₄), tert-butane (C₄), iso-pentane (C₅), neo-pentane (C₆).

Examples of alkenes include, but are not limited to, ethene (C₂), propene (C₃), butene (C₄), pentene (C₅), hexene (C₆).

Examples of alkynes include, but are not limited to, ethine (acetylene) (C₂), propyne (C₃), butyne (C₄), pentyne (C₅), hexyne (C₆).

Examples of (monocyclic) cycloalkanes include, but are not limited to, cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇), methylcyclohexane (C₇), dimethylcyclohexane (C₈), menthane (C₁₀).

Examples of (polycyclic)cycloalkanes include, but are not limited to, thujane (C₁₀), carane (C₁₀), pinane (C₁₀), bornane (C₁₀), norcarane (C₇), norpinane (C₇), norbornane (C₇), adamantane (C₁₀), decalin (decahydronaphthalene) (C₁₀).

Examples of (monocyclic) cycloalkenes include, but are not limited to, cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇), methylcyclohexene (C₇), dimethylcyclohexene (C₈).

Examples of (polycyclic) cycloalkenes include, but are not limited to, camphene (C₁₀), limonene (C₁₀), pinene (C₁₀).

Examples of carboarenes include, but are not limited to, benzene (C₆), naphthalene (C₁₀), azulene (C₁₀), anthracene (C₁₄), phenanthrene (C₁₄), naphthacene (C₁₈), pyrene (C₁₆).

Examples of carboarenes which comprise fused rings, at least one of which is an aromatic ring, include, but are not limited to, indane (e.g., 2,3-dihydro-1H-indene) (C₉), indene (C₉), isoindene (C₉), tetraline (1,2,3,4-tetrahydronaphthalene (C₁₀), acenaphthene (C₁₂), fluorene (C₁₃), phenalene (C₁₃); acephenanthrene (C₁₅), aceanthrene (C₁₆), cholanthrene (C₂₀).

Examples of (monocyclic) heterarenes include, but are not limited to:

N₁: pyrrole (azole) (C₅), pyridine (azine) (C₆);

O₁: furan (oxole) (C₅);

S₁: thiophene (thiole) (C₅);

N₁O₁: oxazole (C₅), isoxazole (C₅), isoxazine (C₆);

N₂O₁: oxadiazole (furazan) (C₅);

N₃O₁: oxatriazole (C₅);

N₁S₁: thiazole (C₅), isothiazole (C₅);

N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);

N₃: triazole (C₅), triazine (C₆); and,

N₄: tetrazole (C₅).

Examples of heterocycles (some of which are also heteroarenes) which comprise fused rings, include, but are not limited to:

C₉heterocycles group (with 2 fused rings): benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);

C₁₀heterocycles (with 2 fused rings): chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);

C₁₁heterocyles (with 2 fused rings): benzodiazepine (N₂);

C₁₃heterocycles (with 3 fused rings): carbazole (N₁), dibenzofuran (O₁), dibenzothiophene (S₁), carboline (N₂), perimidine (N₂), pyridoindole (N₂);

C₁₄heterocycles (with 3 fused rings): acridine (N₁), xanthene (O₁), thioxanthene (S₁), oxanthrene (O₂), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂).

Heterocycles (including heteroarenes) which have a nitrogen ring atom in the form of an —NH— group may be N-substituted, that is, as —NR—. For example, pyrrole may be N-methyl substituted, to give N-methylpyrrole. Examples of N-substitutents include, but are not limited to C₁₋₇alkyl, C₃₋₂₀heterocyclyl, C₅₋₂₀aryl, and acyl groups.

Heterocycles (including heteroarenes) which have a nitrogen ring atom in the form of an —N═ group may be substituted in the form of an N-oxide, that is, as —N(→O)═ (also denoted —N⁺(→O⁻)═). For example, quinoline may be substituted to give quinoline N-oxide; pyridine to give pyridine N-oxide; benzofurazan to give benzofurazan N-oxide (also known as benzofuroxan).

Cyclic compounds may additionally bear one or more oxo (═O) groups on ring carbon atoms.

Monocyclic examples of such compounds include, but are not limited to:

C₅: cyclopentanone, cyclopentenone, cyclopentadienone;

C₆: cyclohexanone, cyclohexenone, cyclohexadienone;

O₁: furanone (C₅), pyrone (C₆);

N₁: pyrrolidone (pyrrolidinone) (C₅), piperidinone (piperidone) (C₆), piperidinedione (C₆);

N₂: imidazolidone (imidazolidinone) (C₅), pyrazolone (pyrazolinone) (C₅), piperazinone (C₆), piperazinedione (C₆), pyridazinone (C₆), pyrimidinone (C₆) (e.g., cytosine), pyrimidinedione (C₆) (e.g., thymine, uracil), barbituric acid (C₆);

N₁S₁: thiazolone (C₅), isothiazolone (C₅);

N₁O₁: oxazolinone (C₅).

Polycyclic examples of such compounds include, but are not limited to:

C₉: indenedione;

C₁₀: tetralone, decalone;

C₁₄: anthrone, phenanthrone;

N₁: oxindole (C₉);

O₁: benzopyrone (e.g., coumarin, isocoumarin, chromone) (C₁₀);

N₁O₁: benzoxazolinone (C₉), benzoxazolinone (C₁₀);

N₂: quinazolinedione (C₁₀); benzodiazepinone (C₁₁); benzodiazepinedione (C₁₁);

N₄: purinone (C₉) (e.g., guanine).

Still more examples of cyclic compounds which bear one or more oxo (═O) groups on ring carbon atoms include, but are not limited to, those derived from:

cyclic anhydrides (—C(═O)—O—C(═O)— in a ring), including but not limited to maleic anhydride (C₅), succinic anhydride (C₅), and glutaric anhydride (C₆);

cyclic carbonates (—O—C(═O)—O— in a ring), such as ethylene carbonate (C₅) and 1,2-propylene carbonate (C₅);

imides (—C(═O)—NR—C(═O)— in a ring), including but not limited to, succinimide (C₅), maleimide (C₅), phthalamide, and glutarimide (C₆);

lactones (cyclic esters, —O—C(═O)— in a ring), including, but not limited to, β-propiolactone, γ-butyrolactone, δ-valerolactone (2-piperidone), and ε-caprolactone;

lactams (cyclic amides, —NR—C(═O)— in a ring), including, but not limited to, β-propiolactam (C₄), γ-butyrolactam (2-pyrrolidone) (C₅), δ-valerolactam (C₆), and ε-caprolactam (C₇);

cyclic carbamates (—O—C(═O)—NR— in a ring), such as 2-oxazolidone (C₅);

cyclic ureas (—NR—C(═O)—NR— in a ring), such as 2-imidazolidone (C₅) and pyrimidine-2,4-dione (e.g., thymine, uracil) (C₆).

Carrier Polymer: Some Preferred Hydrophobic Moieties: Monofunctional

In one embodiment, exactly one, one or more, more than one, or all, of the hydrophobic moieties are selected from moieties derived from compounds which have a hydrophobic group, and bear exactly one, one or more, or more than one reactive functional group(s).

In one embodiment, exactly one, one or more, more than one, or all, of the hydrophobic moieties are selected from moieties derived from compounds of the formula:
Q-G¹
wherein: G¹ is a hydrophobic group; and Q is independently a reactive functional group.

In one embodiment, exactly one of the hydrophobic moieties is so selected.

In one embodiment, one or more of the hydrophobic moieties is so selected.

In one embodiment, more than one of the hydrophobic moieties are so selected.

In one embodiment, all of the hydrophobic moieties are so selected.

The hydrophobic group, G¹, is derived, for example, from a compound as described above for hydrophobic moieties.

Such compounds are useful, for example, when the hydrophobic group is to be appended to, or pendant from, the carrier polymer, e.g., the backbone of the carrier polymer.

Carrier Polymer: Some Preferred Hydrophobic Moieties: Bifunctional

In one embodiment, exactly one, one or more, more than one, or all, of the hydrophobic moieties are selected from moieties derived from compounds which have a hydrophobic group, and bear exactly two, two or more, or more than two reactive functional groups.

In one embodiment, exactly one, one or more, more than one, or all, of the hydrophobic moieties are selected from moieties derived from compounds of the formula:
Q-G²-Q
wherein: G² is a hydrophobic group; and each Q is independently a reactive functional group.

In one embodiment, exactly one of the hydrophobic moieties is so selected.

In one embodiment, one or more of the hydrophobic moieties is so selected.

In one embodiment, more than one of the hydrophobic moieties are so selected.

In one embodiment, all of the hydrophobic moieties are so selected.

The hydrophobic group, G², is derived, for example, from a compound as described above for hydrophobic moieties.

Such compounds are useful, for example, when the hydrophobic group is to be part of, or form part of, the backbone of the carrier polymer; or where the hydrophobic group is to be appended to, or pendant from, the carrier polymer, e.g., the backbone of the carrier polymer, but is to be further modified.

Carrier Polymer: Some Preferred Hydrophobic Moieties:

Reactive Functional Groups, Q

In one embodiment, each reactive functional group, Q, is independently selected from:

(i) reactive acyl groups;

(ii) hydroxy groups (i.e., —OH); and,

(iii) amino groups (e.g., —NH₂, —NHR, —NR₂, etc.).

In one embodiment, each reactive functional group, Q, is independently reactive acyl group, denoted herein as —C(═O)X.

Examples of reactive acyl groups include, but are not limited to:

(a) carboxylic acid, where X is —H;

(b) acyl halides, where X is halogen (e.g., Cl, Br, I);

(c) acid anhydrides, where X is —OC(═O)R^{AN}, wherein R^{AN} is an acid anhydride substituent, and —C(═O)OC(═O)R^{AN} is an acid anhydride group suitable for reaction, for example, with an amino group;

(d) esters, where X is —OR^{E}, wherein R^{E} is an ester substituent, and C(O)OR^{E} is an ester group, for example, an activated ester group suitable for reaction, for example, with an amino group.

In one embodiment, each Q is independently selected from: (a).

In one embodiment, each Q is independently selected from: (b).

In one embodiment, each Q is independently selected from: (c).

In one embodiment, each Q is independently selected from: (d).

Carrier Polymer: Some Preferred Hydrophobic Moieties: Reactive Acyl Groups

In one embodiment, exactly one, one or more, more than one, or all, of the hydrophobic moieties are selected from moieties derived from compounds of the formula: 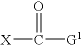
wherein: G¹ is hydrophobic group, as defined herein; and —C(═O)X is independently a reactive functional group, as defined herein.

In one embodiment, exactly one of the hydrophobic moieties is so selected.

In one embodiment, one or more of the hydrophobic moieties is so selected.

In one embodiment, more than one of the hydrophobic moieties are so selected.

In one embodiment, all of the hydrophobic moieties are so selected.

In one embodiment, exactly one, one or more, more than one, or all, of the hydrophobic moieties are selected from moieties derived from compounds of the formula: 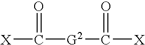
wherein: G² is hydrophobic group, as defined herein; and each —C(═O)X is independently a reactive functional group, as defined herein.

In one embodiment, exactly one of the hydrophobic moieties is so selected.

In one embodiment, one or more of the hydrophobic moieties is so selected.

In one embodiment, more than one of the hydrophobic moieties are so selected.

In one embodiment, all of the hydrophobic moieties are so selected.

Carrier Polymer: Some Preferred Hydrophobic Moieties:

Hydrophobic Groups: Carboarenes

In one embodiment, G¹ and/or G² is a hydrophobic group derived from a carboarene having from 6 to 20 ring carbon atoms.

In one embodiment, G¹ and/or G² is a hydrophobic group derived from benzene (C₆).

In one embodiment, G² is selected from: 1,2-phenylene, 1,3-phenylene, and 1,4-phenylene.

In one embodiment, G² is 1,3-phenylene.

In one embodiment, exactly one, one or more, more than one, or all, of the hydrophobic moieties are selected from those derived from the following compounds: 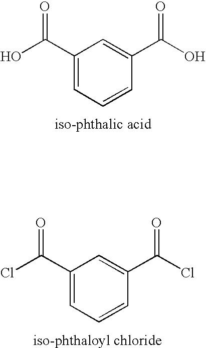
Carrier Polymer: Some Preferred Hydrophobic Moieties:
Hydrophobic Groups: Alkanes and the Like

In one embodiment, G¹ and/or G² is a hydrophobic group derived from:

(a) an alkane, having from 1 to 20 carbon atoms;

(b) an alkene or an alkyne having from 2 to 20 carbon atoms; or,

(c) a cycloalkane, a cylcoalkene, or a cycloalkyne, having from 3 to 20 carbon atoms.

In one embodiment, G¹ and/or G² is a hydrophobic group derived from:

(a) an alkane, having from 1 to 20 carbon atoms; or,

(b) an alkene or an alkyne having from 2 to 20 carbon atoms.

In one embodiment, G¹ and/or G² is a hydrophobic group derived from an alkane having from 1 to 20 carbon atoms.

In one embodiment, G¹ and/or G² is a hydrophobic group derived from an alkane having from 1 to 10 carbon atoms.

In one embodiment, G¹ and/or G² is a hydrophobic group derived from a linear alkane having from 1 to 20 carbon atoms.

In one embodiment, G¹ and/or G² is a hydrophobic group derived from a linear alkane having from 1 to 10 carbon atoms.

In one embodiment, G² is —(CH₂)ₚ—, wherein p is an integer from 1 to 10.

In one embodiment, G² is —(CH₂)ₚ—, wherein p is an integer from 1 to 6.

In one embodiment, G² is —(CH₂)ᵣ—. In one embodiment, G² is —(CH₂)₅—.

In one embodiment, G² is —(CH₂)₄—. In one embodiment, G² is —(CH₂)₃—.

In one embodiment, G² is —(CH₂)₂—. In one embodiment, G² is —(CH₂)—.

In one embodiment, exactly one, one or more, more than one, or all, of the hydrophobic moieties are selected from those derived from the following compounds, wherein p is as defined above (e.g., malonic acid, succinic acid, glutaric acid, adipic acid, pimelic acid, and suberic acid; and dihalides thereof (e.g., dichlorides thereof)): 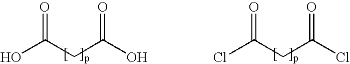
Carrier Polymer: Some Preferred Hydrophobic Moieties:
Hydrophobic Groups: Heteroarenes

In one embodiment, G¹ and/or G² is a hydrophobic group derived from:

(g) an alkane, an alkene, an alkyne, a cycloalkane, a cylcoalkene, or a cycloalkyne, as defined above, attached to a carboarene as defined above;

(h) an alkane, an alkene, an alkyne, a cycloalkane, a cylcoalkene, or a cycloalkyne, as defined above, attached to a heteroarene as defined above; or,

(i) an alkane, an alkene, an alkyne, a cycloalkane, a cylcoalkene, or a cycloalkyne, as defined above, attached to a heterocycle as defined above.

In one embodiment, G¹ and/or G² is a hydrophobic group derived from:

(h) an alkane, an alkene, an alkyne, a cycloalkane, a cylcoalkene, or a cycloalkyne, as defined above, attached to a heteroarene as defined above.

In one embodiment, G¹ and/or G² is a hydrophobic group derived from:

an alkane, an alkene, or an alkyne, as defined above, attached to a heteroarene as defined above.

In one embodiment, G¹ and/or G² is a hydrophobic group derived from tryptophan and tryptophan analogs, including but not limited to, 5-hydroxy-tryptophan, tryptamine, desaminotryptophan.

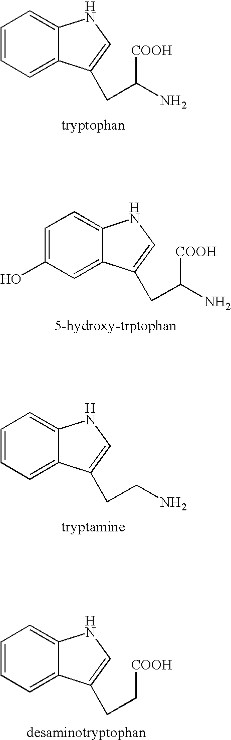

In one embodiment, G¹ and/or G² is a hydrophobic group derived from:

(g) an alkane, an alkene, an alkyne, a cycloalkane, a cylcoalkene, or a cycloalkyne, as defined above, attached to a carboarene as defined above.

In one embodiment, G¹ and/or G² is a hydrophobic group derived from:

an alkane, an alkene, or an alkyne, as defined above, attached to a carboarene as defined above.

In one embodiment, G¹ and/or G² is a hydrophobic group derived from tyrosine and tyrosine analogs, including but not limited to, meta-tyrosine, ortho-tyrosine, desaminotyrosine, tyramine. 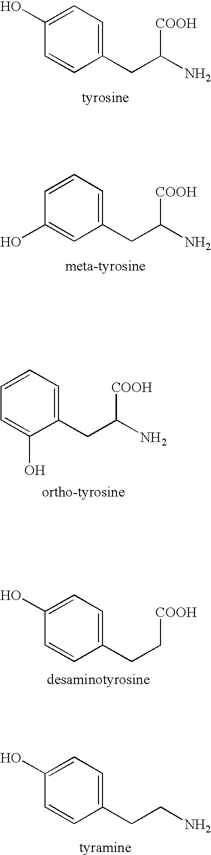
Carrier Polymer: Hydrophilic Moieties: Ionic

In general, ionic moieties are more hydrophilic than the corresponding non-ionic species. In general, when a moiety changes from a neutral species to a charged species, its hydrophilicity is increased; conversely, when a moiety changes from a charged species to a neutral species, its hydrophilicity is decreased. This effect (i.e., a change in hydrophilicity upon charge formation/charge neutralization) often contributes to the pH responsive properties of the carrier polymer.

Thus, unless otherwise specified, a reference to a hydrophilic moiety or region is intended to be a reference to a “pH responsive” hydrophilic moiety or region, that is, a hydrophilic moiety or region which contributes to the pH responsive nature of the carrier polymer of which it forms a part.

For example, the carrier polymer may comprise other moieties, regions, etc. (see below) which have hydrophilic character, but which are not pH-responsive, that is, do not contribute to the pH responsive nature of the carrier polymer of which it forms a part. For example, water-solubilizing groups (see below), such as PEG, are hydrophilic, but do not contribute to the pH responsive nature of the carrier polymer of which it forms a part.

In one embodiment, exactly one, one or more, more than one, or all, of the hydrophilic moieties is ionic (e.g., anionic, cationic, zwitterionic), that is, bears a charge, or is capable of bearing a charge, in an aqueous environment, and wherein that charge is neutralized above a predetermined pH, or below a predetermined pH, which predetermined pH falls in the range of about pH 4 to about pH 9.

In one embodiment, the charge is anionic.

In one embodiment, the charge is cationic.

In one embodiment, exactly one, one or more, more than one, or all, of the hydrophilic moieties bears a chargeable group (that is, a group which bears a charge, or is capable of bearing a charge, in an aqueous environment), or a salt thereof, wherein the pH, at which the chargeable group exists in equilibrium with equal amounts (e.g., concentration) of the electrically neutral form and the ionic form, is in the range of about pH 4 to about pH 9.

In one embodiment, the chargeable group, when charged, is anionic.

In one embodiment, the chargeable group, when charged, is cationic.

In one embodiment, exactly one, one or more, more than one, or all, of the hydrophilic moieties is a weak Bronsted acid or a weak Bronsted base.

Bronsted acids donate protons (e.g., HA→A⁻+H⁺). Bronsted bases accept protons (e.g., B+H⁺→BH⁺). In aqueous solution, Bronsted acids (HA) and Bronsted bases (B) exist in equilibrium with their conjugate bases (A⁻) and conjugate acids (BH⁺), respectively. These aqueous equilibria are characterised by equilibrium constants, denoted Kₐ and K_{b}, respectively, which are often reported as pKₐ and pK_{b} values, typically for a temperature of 25° C. Kₐ values are often referred to as “dissociation constants.” Weak Bronsted acids which are useful in the present invention are characterized by pKₐ values in the range of about 3 to about 8; about 3 to about 7; about 4.5 to about 6.7; about 5.0 to about 6.5.

In one embodiment, the weak Bronsted acid is a carboxylic acid.

Weak Bronsted bases which are useful in the present invention are characterized by pKₐ values in the range of about 5 to about 12. In one embodiment, the range is about 6 to about 9; about 7 to about 8; about 6.5 to about 7.5

Note that the zwitterionic nature of amino acids causes the pKₐ of the carboxylic acid group to be relatively low, e.g., about 2 (the pKₐ for the carboxylic acid group of free lysine, at 25° C., is about 2.18; see, e.g., Pine, 1988, Organic Chemistry, 5th Edition, publisher: McGraw-Hill), as compared to about 4-5 for free carboxylic acids (e.g., the pKₐ of formic acid, at 25° C., is about 4.76). When amino acids are polymerized, the zwitterionic nature is lost, since the carboxylic acid group and amino group are converted to an amide. Also, the pKₐ of the carboxylic acid groups within a polymer are usually higher than for the free monomer, due to an increase in electrostatic potential between carboxylate ions as the degree is ionization is increased. For example citric acid has three carboxylic acid groups with increasing pKₐ values; each pKₐ is distinct. In the case of a polymer with hundreds or thousands of carboxylic acid groups, they “blend” to give a broad band of pKₐ values.

Note that, sulfonic acids (R—S(═O)₂OH) and phosphonic acids (R—P(═O)(OH)₂) fall outside the ranges which are useful in the present invention.

Carrier Polymer: Hydrophilic Moieties: Carboxylic Acids

In one embodiment, exactly one, one or more, more than one, or all, of the hydrophilic moieties bears a carboxylic acid group (—C(═O)OH) or a salt thereof.

In one embodiment, exactly one, one or more, more than one, or all, of the hydrophilic moieties bears a carboxylic acid group (—C(═O)OH) or a salt thereof, wherein the pH at which the carboxylic acid group exists in equilibrium with equal amounts (e.g., concentration) of the neutral acid form (—C(═O)OH) and the anionic base from (—C(═O)O⁻) is in the range of about pH 3 to about pH 8. In one embodiment, the range is about 3 to about 7; about 4.5 to about 6.7; about 5.0 to about 6.5.

Carrier Polymer: Hydrophilic Moieties: Amines

In one embodiment, exactly one, one or more, more than one, or all, of the hydrophilic moieties bears an amino base group (that is, an amino group that is a Bronsted base) selected from:

a primary amino group (—NH₂);

a pendant secondary amino group (—NHR);

a non-pendant non-cyclic secondary amino group (—NH—);

a cyclic secondary amino group (—NH—) (e.g., which forms part of a cyclic structure, as in piperidine);

a pendant tertiary amino group (—NR₂);

a non-pendant non-cyclic tertiary amino group (—NR— or —N═);

a cyclic tertiary amino group (—NR— or —N═) (e.g., which forms part of a cyclic structure, as in pyridine);

or a salt thereof.

In one embodiment, the above selection is from: a primary amino group (—NH₂), a pendant secondary amino group (—NHR), a pendant tertiary amino group (—NR₂), or a salt thereof.

In one embodiment, exactly one, one or more, more than one, or all, of the hydrophilic moieties bears an amino base group (as defined above) or a salt thereof, wherein the pH at which the amino base group exists in equilibrium with equal amounts (e.g., concentration) of the neutral base form (e.g., —NH₂) and the cationic acid from (e.g., —NH₃⁺) is in the range of about pH 4 to about pH 9. In one embodiment, the range is about pH 5.0 to about pH 9.0; about pH 6.0 to about pH 9.0; about pH 7.0 to about pH 9.0.

For example, the pKₐ for the amino group (—N═) of pyridine, at 25° C., is about 5.25.

For example, pyridyl groups having are relatively hydrophobic when electrically neutral (in base form), but are relatively hydrophilic when cationic (in acid conjugate form).

Carrier Polymer: Some Preferred Hydrophilic Moieties: Carboxylic Acids

In one embodiment, exactly one, one or more, more than one, or all, of the hydrophilic moieties are selected from moieties of the formula: 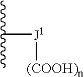
wherein: J¹ is core group; and n is an integer from 1 to 4.

In one embodiment, exactly one, one or more, more than one, or all, of the hydrophilic moieties are selected from moieties of the formula: 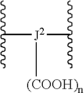
wherein: J² is core group; and n is an integer from 1 to 4.

In one embodiment, n is 1, 2, 3, or 4; n is 1, 2, or 3; n is 1 or 2; n is 4; n is 3; n is 2; n is 1.

The core groups, J¹ and J², are independently derived, for example, from a compound as described above under the heading “Carrier Polymer: Some Preferred Hydrophobic Moieties.”

In one embodiment, exactly one of the hydrophilic moieties is so selected.

In one embodiment, one or more of the hydrophilic moieties is so selected.

In one embodiment, more than one of the hydrophilic moieties are so selected.

In one embodiment, all of the hydrophilic moieties are so selected.

Carrier Polymer: Some Preferred Hydrophilic Moieties: Carboxylic Acids: Monofunctional

In one embodiment, exactly one, one or more, more than one, or all, of the hydrophilic moieties are selected from moieties derived from compounds of the formula: 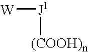
wherein: J¹ and n are as defined above; and W is independently a reactive functional group.

In one embodiment, exactly one of the hydrophilic moieties is so selected.

In one embodiment, one or more of the hydrophilic moieties is so selected.

In one embodiment, more than one of the hydrophilic moieties are so selected.

In one embodiment, all of the hydrophilic moieties are so selected.

Such compounds are useful, for example, when the hydrophilic group is to be appended to, or pendant from, the carrier polymer, e.g., the backbone of the carrier polymer.

Carrier Polymer: Some Preferred Hydrophilic Moieties: Carboxylic Acids: Bifunctional

In one embodiment, exactly one, one or more, more than one, or all, of the hydrophilic moieties are selected from moieties derived from compounds which have one, one or more, or more than one carboxylic acids group(s), —C(═O)OH, or a salt thereof, and additionally bear exactly two, two or more, or more than two reactive functional group(s).

In one embodiment, exactly one, one or more, more than one, or all, of the hydrophilic moieties are selected from moieties derived from compounds of the formula: 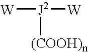
wherein: J² and n are as defined above; and each W is independently a reactive functional group.

In one embodiment, exactly one of the hydrophilic moieties is so selected.

In one embodiment, one or more of the hydrophilic moieties is so selected.

In one embodiment, more than one of the hydrophilic moieties are so selected.

In one embodiment, all of the hydrophilic moieties are so selected.

Such compounds are useful, for example, when the hydrophilic group is to be part of, or form part of, the backbone of the carrier polymer; or where the hydrophilic group is to be appended to, or pendant from, the carrier polymer, e.g., the backbone of the carrier polymer, but is to be further modified.

Carrier Polymer: Some Preferred Hydrophilic Moieties:

Reactive Functional Groups, W

In one embodiment, each reactive functional group, W, is independently selected from:

(i) reactive acyl groups (as defined above);

(ii) hydroxy (i.e., —OH); and,

(iii) amino groups (e.g., —NH₂, —NHR, —NR₂, etc.).

In one embodiment, each W is independently selected from: (ii) and (iii).

In one embodiment, each W is independently selected from: (ii).

In one embodiment, each W is independently selected from: (iii).

In one embodiment, each W is independently selected from: —OH and —NH₂.

In one embodiment, each W is independently —OH.

In one embodiment, each W is independently —NH₂.

In one embodiment, wherein there are two reactive functional groups, W: one is —OH and one is —NH₂.

Carrier Polymer: Some Preferred Hydrophilic Moieties:

Core Groups: Alkanes and the Like

In one embodiment, J¹ and/or J² is independently a core group derived from:

(a) an alkane, having from 1 to 20 carbon atoms;

(b) an alkene or an alkyne having from 2 to 20 carbon atoms; or,

(c) a cycloalkane, a cylcoalkene, or a cycloalkyne, having from 3 to 20 carbon atoms.

In one embodiment, J¹ and/or J² is independently a core group derived from:

(a) an alkane, having from 1 to 20 carbon atoms; or,

(b) an alkene or an alkyne having from 2 to 20 carbon atoms.

In one embodiment, J¹ and/or J² is independently a core group derived from an alkane having from 1 to 20 carbon atoms; from 1 to 10 carbon atoms.

In one embodiment, J¹ and/or J² is independently a core group derived from a linear alkane having from 1 to 20 carbon atoms, from 1 to 10 carbon atoms; from 2 to 7 carbon atoms; from 3 to 6 carbon atoms.

In one embodiment, n is 1 and J² is independently selected from:

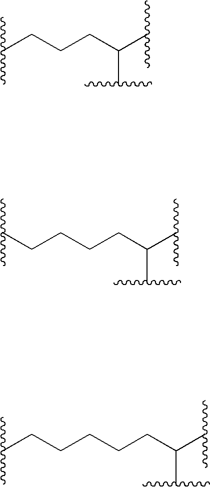

In one embodiment, n is 2 and J² is independently selected from:

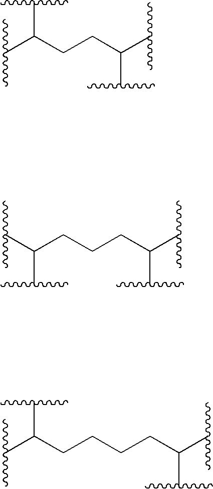

In one embodiment, exactly one, one or more, more than one, or all, of the hydrophilic moieties are selected from moieties of the formula:

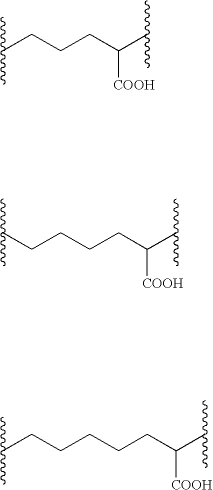

In one embodiment, exactly one, one or more, more than one, or all, of the hydrophilic moieties are selected from moieties of the formula:

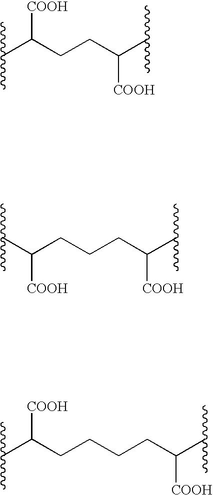

In one embodiment, exactly one, one or more, more than one, or all, of the hydrophilic moieties are independently selected from those derived from amino acids, for example, α-amino acids.

In one embodiment, exactly one, one or more, more than one, or all, of the hydrophilic moieties are independently selected from those derived from di-amino acids, for example, α,ω-di-amino acids.

In one embodiment, exactly one, one or more, more than one, or all, of the hydrophilic moieties are independently selected from those derived from the following compounds: 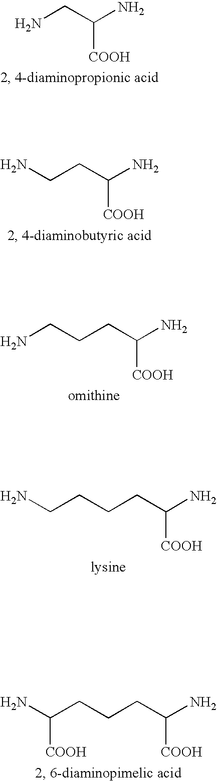
Carrier Polymer: Some Preferred Hydrophilic Moieties:
Core Groups: Arenes and the Like

In one embodiment, J¹ and/or J² is independently a core group derived from:

(g) an alkane, an alkene, an alkyne, a cycloalkane, a cylcoalkene, or a cycloalkyne, as defined above, attached to a carboarene as defined above; or

(h) an alkane, an alkene, an alkyne, a cycloalkane, a cylcoalkene, or a cycloalkyne, as defined above, attached to a heteroarene as defined above.

In one embodiment, the selection is from moieties derived from: (g).

In one embodiment, the selection is from moieties derived from: (h).

In one embodiment, J¹ and/or J² is independently a core group derived from:

an alkane, an alkene, or an alkyne, as defined above, attached to a carboarene as defined above; or

an alkane, an alkene, or an alkyne, as defined above, attached to a heteroarene as defined above.

In one embodiment, J¹ and/or J² is independently a core group derived from:

an alkane, as defined above, attached to a carboarene as defined above; or

an alkane, as defined above, attached to a heteroarene as defined above.

In one embodiment, J¹ and/or J² is independently a core group derived from: an alkane, as defined above, attached to a carboarene as defined above.

In one embodiment, J¹ and/or J² is independently a core group derived from: an alkane, as defined above, attached to a heteroarene as defined above.

In one embodiment, n is 1 and J² is independently selected from:

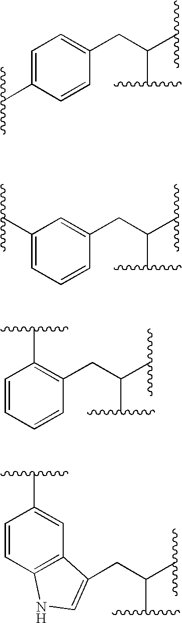

In one embodiment, exactly one, one or more, more than one, or all, of the hydrophilic moieties are selected from moieties of the formula:

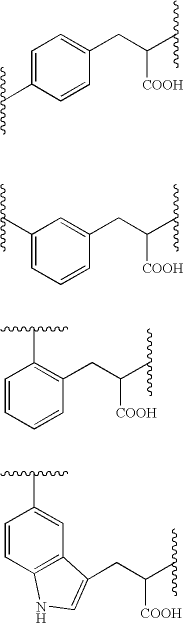

In one embodiment, exactly one, one or more, more than one, or all, of the hydrophilic moieties are independently selected from those derived from the following compounds: 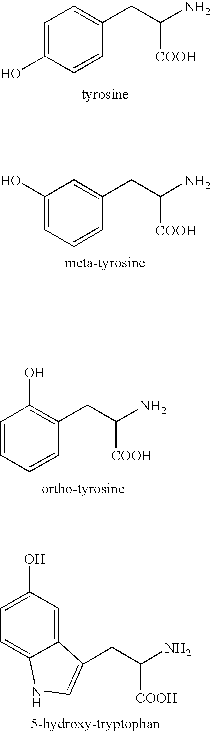
Carrier Polymer: Some Preferred Carrier Polymers: Co-Polymers

In one embodiment, the carrier polymer is a co-polymer of: (a) a monomer providing a hydrophobic moiety (e.g., as defined herein); and (b) a monomer providing a hydrophilic moiety (e.g., as defined herein).

Each compatible combination of monomers providing a hydrophobic moiety (e.g., as defined herein) and monomers providing a hydrophilic moiety (e.g., as defined herein) is included as if it was explicitly recited.

For example, in one embodiment, the carrier polymer is a co-polymer of:

(a) a monomer selected from iso-phthalic acid and iso-phthaloyl chloride; and

(b) a monomer selected from 2,4-diaminopropionic acid; 2,4-diaminobutyric acid; ornithine; lysine; or 2,6-diaminopimelic acid.

For example, in one embodiment, the carrier polymer is poly(lysine iso-phthalamide).

Carrier Polymer: Modification of a Homopolymer

In one embodiment, hydrophobic and hydrophilic regions may be formed from the same, or similar, monomer components.

For example, in a lysine polymer or co-polymer, lysine units (linked via the amino groups) may be left unmodified, having a pendant carboxylic acid group (—COOH), which has hydrophilic properties, or it may be “hydrophobically-modified” so as to have, for example, a pendant ester group (—COOR) or a pendant amide group (—CONR₂), wherein the R group (or one or both of each of the two R groups) is selected to provide hydrophobic properties. Examples of such modifications are shown below.

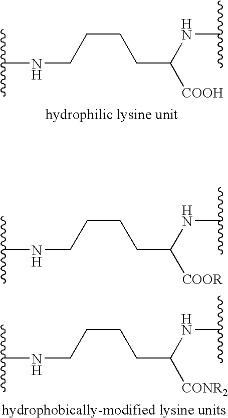

For example, some lysine units may be converted to amides by reaction with suitable amines, such as, for example, phenylalanine and norleucine. Examples of such modifications are shown below.

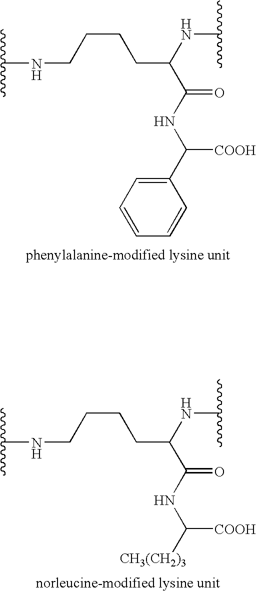

Examples of other polymer units which may be modified in this way include, but are not limited to, β-aspartic acid units and malic acid units. Examples of such modifications are shown below.

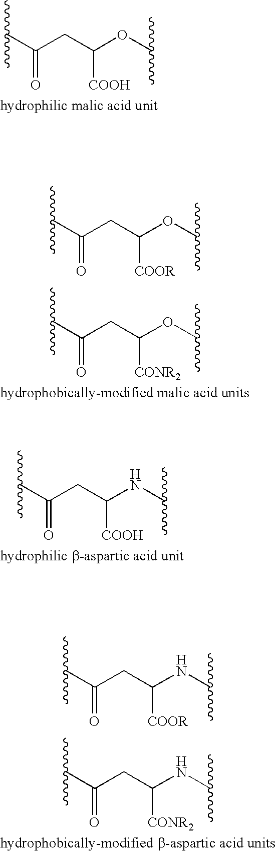

In one embodiment, exactly one, one or more, more than one, or all, of the hydrophobic moieties of the carrier polymer are independently a hydrophobically-modified hydrophilic moiety.

In one embodiment, the hydrophilic moiety of the hydrophobically-modified hydrophilic moiety bears a pendant carboxylic acid group, for example, as described above (e.g., as in lysine, β-aspartic acid, malic acid, and the like) that has been derivatized to bear a hydrophobic group.

In one embodiment, the hydrophilic moiety of the hydrophobically-modified hydrophilic moiety bears a pendant carboxylic acid group, for example, as described above (e.g., as in lysine, β-aspartic acid, malic acid, and the like) that has been derivatized to bear a hydrophobic group, for example, by reaction with a hydrophobic modifier, e.g., a hydrophobic amino acid (e.g., alanine, valine, norvaline, leucine, isoleucine, norleucine, phenylalanine, phenylglycine, tyrosine, tryptophan) to form a hydrophobic pendant amide of the hydrophobically-modified hydrophilic moiety.

In one embodiment, the hydrophilic moiety of the hydrophobically-modified hydrophilic moiety is selected from lysine, β-aspartic acid, and malic acid.

In one embodiment, the hydrophobically-modified hydrophilic moiety is selected from: alanine-, valine-, norvaline-, leucine-, isoleucine-, norleucine-, phenylalanine-, phenylglycine-, tyrosine-, and tryptophan-modified lysine, β-aspartic acid, and malic acid.

In one embodiment, exactly one, one or more, more than one, or all, of the hydrophilic moieties (of the carrier polymer) correspond to (is the same as) exactly one, one or more, more than one, or all, of the hydrophilic moieties of the hydrophobically-modified hydrophilic moieties (which are hydrophobic moieties of the carrier polymer).

For example, as in hydrophobically-modified polylysine, all of the hydrophilic moieties (lysine) correspond to all of the hydrophilic moieties (lysine) of the hydrophobically-modified hydrophilic moieties (hydrophobically-modified lysine).

For example, as in hydrophobically-modified poly(aspartic acid), all of the hydrophilic moieties (aspartic acid) correspond to all of the hydrophilic moieties (aspartic acid) of the hydrophobically-modified hydrophilic moieties (hydrophobically-modified aspartic acid).

In one embodiment, exactly one, one or more, more than one, or all, of the hydrophilic moieties (of the carrier polymer) is selected from lysine, β-aspartic acid, and malic acid, and exactly one, one or more, more than one, or all, of the hydrophilic moieties of the hydrophobically-modified hydrophilic moieties (which are hydrophobic moieties of the carrier polymer) is selected from hydrophobically-modified lysine, β-aspartic acid, and malic acid.

In one embodiment, exactly one, one or more, more than one, or all, of the hydrophilic moieties (of the carrier polymer) is selected from lysine, and exactly one, one or more, more than one, or all, of the hydrophilic moieties of the hydrophobically-modified hydrophilic moieties (which are hydrophobic moieties of the carrier polymer) is selected from hydrophobically-modified lysine.

In one embodiment, exactly one, one or more, more than one, or all, of the hydrophilic moieties (of the carrier polymer) is selected from β-aspartic acid, and exactly one, one or more, more than one, or all, of the hydrophilic moieties of the hydrophobically-modified hydrophilic moieties (which are hydrophobic moieties of the carrier polymer) is selected from hydrophobically-modified β-aspartic acid.

In one embodiment, exactly one, one or more, more than one, or all, of the hydrophilic moieties (of the carrier polymer) is selected from malic acid, and exactly one, one or more, more than one, or all, of the hydrophilic moieties of the hydrophobically-modified hydrophilic moieties (which are hydrophobic moieties of the carrier polymer) is selected from hydrophobically-modified malic acid

In one embodiment, the ratio of the hydrophilic moieties (of the carrier polymer) to the corresponding hydrophobically-modified hydrophilic moieties (e.g., the ratio of lysine to norleucine-modified lysine), by number, for the carrier polymer is from about 0.2 (1:5) to about 5 (5:1).

In one embodiment, the ratio is from about 0.4 (2:5) to about 2.5 (5:2).

In one embodiment, the ratio is from about 0.4 (2:5) to about 0.6 (3:5).

In one embodiment, the ratio is about 0.5 (1:2).

In one embodiment, the ratio is from about 0.8 (8:10) to about 1.2 (12:10).

In one embodiment, the ratio is about 1 (1:1).

In one embodiment, the ratio is from about 1.8 (18:10) to about 2.2 (22:10).

In one embodiment, the ratio is about 2 (2:1).

Carrier Polymer: Some Preferred Carrier Polymers: Modified Homopolymers

In one embodiment, the carrier polymer is a hydrophobically-modified polymer of a monomer providing a hydrophilic moiety (e.g., as defined herein), which polymer has been modified to have hydrophobically-modified monomer units.

Each compatible combination of monomers providing a hydrophilic moiety (e.g., as defined herein) and hydrophobic modifiers (e.g., as defined herein) is included as if it was explicitly recited.

For example, in one embodiment, the carrier polymer is hydrophobically-modified poly(lysine), poly(β-aspartic acid), or poly(malic acid), which polymer has been modified to have hydrophobically-modified monomer units selected from alanine-, valine-, norvaline-, leucine-, isoleucine-, norleucine-, phenylalanine-, phenylglycine-, tyrosine-, and tryptophan-modified lysine, β-aspartic acid, and malic acid, respectively.

For example, in one embodiment, the carrier polymer is hydrophobically-modified poly(lysine), which polymer has been modified to have phenylalanine-modified lysine monomer units.

Carrier Polymer: Other Components

As discussed above, the carrier polymer has hydrophobic regions (comprising hydrophobic moieties) and hydrophilic regions (comprising hydrophilic moieties), and incorporates, or is otherwise associated with, a payload (comprising payload moieties).

Also as discussed above, these moieties may be connected though chemical linkages, such as amide linkages.

In addition, the carrier polymer may further comprise other regions and/or moieties, including but not limited to, spacer groups, water solubilizing groups (such as polyethylene glycol (PEG), poly ethylene oxide (PEO), polyvinyl alcohol (PVA), hydroxylpropylmethyl alcohol (HPMA), and dextran groups), targeting ligands (such as folic acid and galactose), and the like.

For example, some fraction (e.g., 5%) of the pendant carboxylic acid groups of a poly(lysine-iso-phthalic acid) polymer may be modified with PEG (e.g., 5000 MW PEG) to form pendant esters.

Some of these additional regions and/or moieties may have hydrophobic or hydrophilic character; however, they may or may not be pH responsive, that is, they may or may not contribute to the pH responsive nature of the carrier polymer.

Payload

The term “payload,” as used herein, pertains to chemical moieties which are to be delivered, for example, into a living cell, or into the nucleus of a living cell.

The payload may have therapeutic value, for example, as a biologically active agent (therapeutic), or as a species which gives rise, directly or indirectly, to a biologically active agent (therapeutic), which is useful in therapy or treatment (see below).

The payload may have diagnostic value, for example, as a detectable label or as a species which gives rise, directly or indirectly, to a detectable label.

The payload may have both therapeutic value and diagnostic value (e.g., a labelled drug, e.g., a peptide having a radioactive-iodine-labelled tyrosine residue).

The payload may have other value, as an alternative to, or in addition to diagnostic and/or therapeutic value.

Payload Moieties

The carrier polymer incorporates, or is otherwise associated with, a payload.

The payload consists of exactly one, one or more, or more than one (i.e., a plurality of), payload moieties.

In one embodiment, the payload consists of exactly one payload moiety.

In one embodiment, the payload consists of a plurality of payload moieties.

The payload may be homogenous (that is, only one type of payload moiety is present, e.g., a single drug, fluorophore, etc.). Thus, in one embodiment, the plurality of payload moieties are identical.

The payload may be heterogeneous (that is, more than one type of payload moiety is present, e.g., two or more drugs, two members of a FRET pair, etc.). Thus, in one embodiment, the plurality of payload moieties are of two types.

In one embodiment, exactly one, one or more, more than one, or all of the payload moieties may also serve as hydrophobic or hydrophilic moieties. For example, a pendant drug payload moiety may be relatively hydrophobic, and it make serve as, or take the place of, exactly one, one or more, more than one, or all of the hydrophobic moieties of the carrier polymer.

Payload: Payload Moieties: Number of

In one embodiment, the payload consists of exactly one payload moiety.

In one embodiment, the payload consists of from 1 to 10 payload moieties; from 1 to 20 payload moieties; from 1 to 50 payload moieties; from 1 to 100 payload moieties; from 1 to 1000 payload moieties.

In one embodiment, the payload consists of from 2 to 10 payload moieties; from 2 to 20 payload moieties; from 2 to 50 payload moieties; from 2 to 100 payload moieties; from 2 to 1000 payload moieties.

In one embodiment, the payload consists of from 5 to 10 payload moieties; from 5 to 20 payload moieties; from 5 to 50 payload moieties; from 5 to 100 payload moieties; from 5 to 1000 payload moieties.

Payload: Payload Moieties: Number Ratios: Carrier Polymer

In one embodiment, each carrier polymer molecule incorporates, or is otherwise associated with, exactly one, one or more, or more than one payload moieties.

In one embodiment, each carrier polymer molecule incorporates, or is otherwise associated with exactly one payload moiety. This may be suitable, for example, where the payload is relatively large.

In one embodiment, each carrier polymer molecule incorporates, or is otherwise associated with, a plurality of payload moieties. This may be suitable, for example, where the payload is relatively small.

Payload: Payload Moieties: Number Ratios: Carrier Polymer: ≧1

In one embodiment, the ratio of payload moieties to carrier polymer molecules, by number, is about 1 (1:1).

In one embodiment, the ratio of payload moieties to carrier polymer molecules, by number, is more than about 1 (1:1).

In one embodiment, the ratio is from about 1 (1:1) to about 10 (10:1); from about 1 (1:1) to about 100 (100:1); from about 1 (1:1) to about 1000 (1000:1).

Payload: Payload Moieties: Number Ratios: Carrier Polymer: <1

In one embodiment, the ratio of payload moieties to carrier polymer molecules, by number, is less than about 1 (1:1).

In one embodiment, the ratio is from about 0.1 (1:10) to about 1 (1:1); from about 0.01 (1:100) to about 1 (1:1); from about 0.001 (1:1000) to about 1 (1:1); from about 0.0001 (1:10000) to about 1 (1:1).

Payload: Payload Moieties: Molecular Weight

In one embodiment, each payload moiety has a gram molecular weight of from about 50 (e.g., for low molecular weight drugs and the like) to about 10⁴ (e.g., for peptides and the like) to about 10⁸ (e.g., for liposomes and DNA and the like).

In one embodiment, the range is from about 50 to about 1,000; from about 50 to about 5,000; from about 50 to about 10,000; from about 50 to about 20,000; from about 50 to about 30,000.

In one embodiment, the range is from about 100 to about 1,000; from about 100 to about 5,000; from about 100 to about 10,000; from about 100 to about 20,000; from about 100 to about 30,000.

In one embodiment, the range is from about 500 to about 2,000; from about 500 to about 5,000; from about 500 to about 10,000; from about 500 to about 20,000; from about 500 to about 30,000.

In one embodiment, the range is from about 1,000 to about 5,000; from about 1,000 to about 10,000; from about 1,000 to about 20,000; from about 1,000 to about 30,000.

In one embodiment, the range is from about 5,000 to about 10,000; from about 5,000 to about 20,000; from about 5,000 to about 30,000.

In one embodiment, the range is from about 10⁴ to about 10⁶; from about 10⁴ to about 10⁵; from about 10⁵ to about 10⁶.

In one embodiment, the range is from about 10⁵ to about 10⁸; from about 10⁶ to about 10⁸.

Payload: Content by Weight

In one embodiment, the total payload (the combined weight of all payload moieties) accounts for about 0.01% to about 99% by weight of the carrier polymer and the payload (that is, the carrier polymer which incorporates the payload, or the carrier polymer and the otherwise associated payload).

In embodiments where the total payload is relatively large (e.g., more than about 10⁶ Da), the amount is about 50-99%; about 50-95%; about 50-90%.

In embodiments where the total payload is not relatively large (e.g., less than about 10⁶ Da), the amount is about 0.01-50%; about 0.01-40%; about 0.01-30%; about 0.01-20%; about 0.01-10%; about 0.01-5%; about 0.01-1%.

Payload: Payload Moieties: Hydrophobic/Hydrophilic Regions/Moieties:

Number Ratios

In one embodiment, the ratio of payload moieties to hydrophobic regions, by number, for the carrier polymer and the payload (that is, the carrier polymer which incorporates the payload, or the carrier polymer and the otherwise associated payload), is from about 0.001 (1:1000) to about 0.5 (1:2).

In one embodiment, the ratio of payload moieties to hydrophobic moieties, by number, for the carrier polymer and the payload (that is, the carrier polymer which incorporates the payload, or the carrier polymer and the otherwise associated payload), is from about 0.001 (1:1000) to about 0.5 (1:2).

In one embodiment, the ratio of payload moieties to hydrophilic regions, by number, for the carrier polymer and the payload (that is, the carrier polymer which incorporates the payload, or the carrier polymer and the otherwise associated payload), is from about 0.001 (1:1000) to about 0.5 (1:2).

In one embodiment, the ratio of payload moieties to hydrophilic moieties, by number, for the carrier polymer and the payload (that is, the carrier polymer which incorporates the payload, or the carrier polymer and the otherwise associated payload), is from about 0.001 (1:1000) to about 0.5 (1:2).

In one embodiment, the range is from about 0.001 (1:1000) to about 0.01 (1:100).

In one embodiment, the range is from about 0.01 (1:100) to about 0.1 (1:10).

In one embodiment, the range is from about 0.01 (1:100) to about 0.5 (1:2)

Payload: Payload Moieties: “Incorporated”

As discussed above, in one embodiment, the carrier polymer incorporates the payload.

In one embodiment, the payload forms part of the backbone of the carrier polymer.

In one embodiment, the payload is tethered to (e.g., conjugated to, pendant from) the backbone of the carrier polymer, either directly or via linking groups.

For example, in one embodiment, the carrier polymer is poly(lysine iso-phthalamide), and incorporates the payload, and the payload forms part of the backbone of the carrier polymer.

In such embodiments, the compound(s) from which the payload, more particularly, the payload moieties, are derived, bear, or are derivatized so as to bear, exactly one, one or more, exactly two, or two or more, reactive functional groups which permit incorporation into the carrier polymer.

In one embodiment, the payload is tethered to the backbone of the carrier polymer, and the compound(s) from which the payload moieties are derived bear, or are derivatized so as to bear, exactly one, or more than one, reactive functional groups which permit incorporation into the carrier polymer.

In one embodiment, the payload forms part of the backbone of the carrier polymer, and the compound(s) from which the payload moieties are derived bear, or are derivatized so as to bear, exactly two, or two or more, reactive functional groups which permit incorporation into the carrier polymer.

Examples of reactive functional groups which permit incorporation into the carrier polymer include, but are not limited to, for example, amino, hydroxy (including, e.g., hydroxyphenyl), carboxylic acid, esters (e.g., activated esters), halides, acyl halides, chloroformates, isocyanates, and hydrazines.

In one embodiment, the reactive functional groups which permit incorporation into the carrier polymer are selected from: amino, carboxylic acid, esters (e.g., activated esters), and acyl halides.

In one embodiment, the reactive functional groups which permit incorporation into the carrier polymer are selected so that the linkage formed upon incorporation into the carrier polymer is an amide linkage.

In one embodiment, the reactive functional groups which permit incorporation into the carrier polymer are selected from: amino, carboxylic acid, esters (e.g., activated esters), acyl halides, and the linkage formed upon incorporation into the carrier polymer is an amide linkage.

In one embodiment, the reactive functional groups which permit incorporation into the carrier polymer are amino groups, and the linkage formed upon incorporation into the carrier polymer is an amide linkage.

In one embodiment, the reactive functional groups which permit incorporation into the carrier polymer are selected from: carboxylic acid, esters (e.g., activated esters), and acylhalides, and the linkage formed upon incorporation into the carrier polymer is an amide linkage.

Payload: Biologically Active Agents: Examples

In one embodiment, exactly one, one or more, more than one, or all of the payload moieties are, or comprise, biologically active agents (therapeutics) selected from:

(a) drugs, prodrugs, chemo-therapeutics, radio-therapeutics, neutron capture agents, and the like;

(b) peptides; proteins, antibodies, antibody fragments, enzymes, transcription factors, signalling proteins, antisense peptides, zinc fingers, peptide vaccines, and the like; and,

(c) nucleotides, oligonucleotides, plasmids, nucleic acids, and the like.

In one embodiment, the selection is from (a).

In one embodiment, the selection is from (a) and (b).

In one embodiment, the selection is from (b).

In one embodiment, the selection is from (b) and (c).

In one embodiment, the selection is from (c).

For example, in one embodiment, the payload is a neutron capture agent (such boron-containing compound/moiety) which, for example, treats a condition (e.g., cancer) by emitting alpha particles upon irradiation with neutrons.

For example, in one embodiment, the payload is a near-infrared absorbing or emitting chromophore or fluorophore which, for example, treats a condition by causing a local heating effect (hyperthermia), which may itself have a therapeutic effect, or may enhancing the therapeutic effect (e.g., efficacy) of a co-administered drug.

For example, in one embodiment, the payload is a therapeutic or prophylactic peptide, protein, oligonucleotide, or plasmid, which, for example, treats a condition (e.g., cancer).

Payload: Detectable Labels: Examples

In one embodiment, exactly one, one or more, more than one, or all of the payload moieties are, or comprise, detectable labels selected from:

(a) fluorophores (e.g., near-infrared fluorescence (NIRF) labels, such as polymethine dyes, e.g., cyanine dyes; fluorescence resonance energy transfer (FRET) pairs, such as Cy3 and Cy5; Cy5 and Cy7);

(b) chromophores (e.g., methylene blue);

(c) isotopically enriched species (e.g., ¹³C enriched groups);

(d) paramagnetic species (e.g., gadolinium(III), iron(III), manganese(II));

(e) radioactive species (e.g., ⁹⁹Tc);

(f) scintillents and phosphors (e.g., 2,5-diphenyloxazole).

Payload: Payload Moieties: Specific Examples: Fluorophores: Dyes

Examples of payloads which have diagnostic value, by virtue of possessing a fluorophore, include dyes and derivatives thereof, including, but not limited to, cyanine dyes and derivatives thereof. See, for example, Reddington, 1998.

In one embodiment, exactly one, one or more, more than one, or all of the payload moieties is a cyanine dye or a derivative thereof.

Examples of suitable cyanine dye and derivatives thereof include:

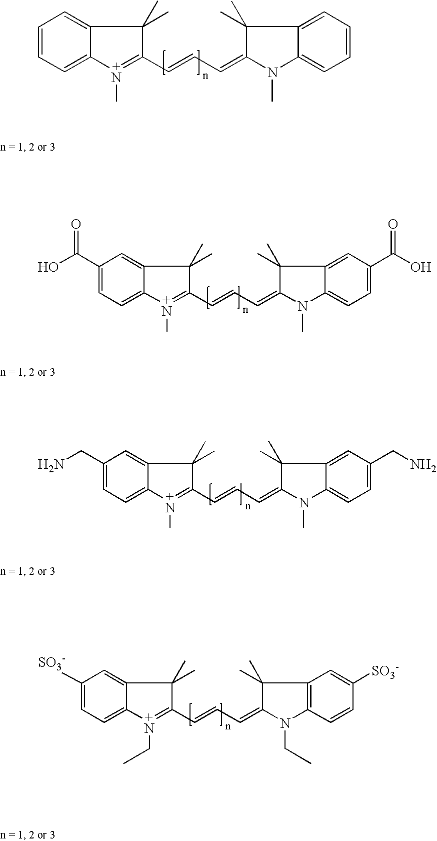

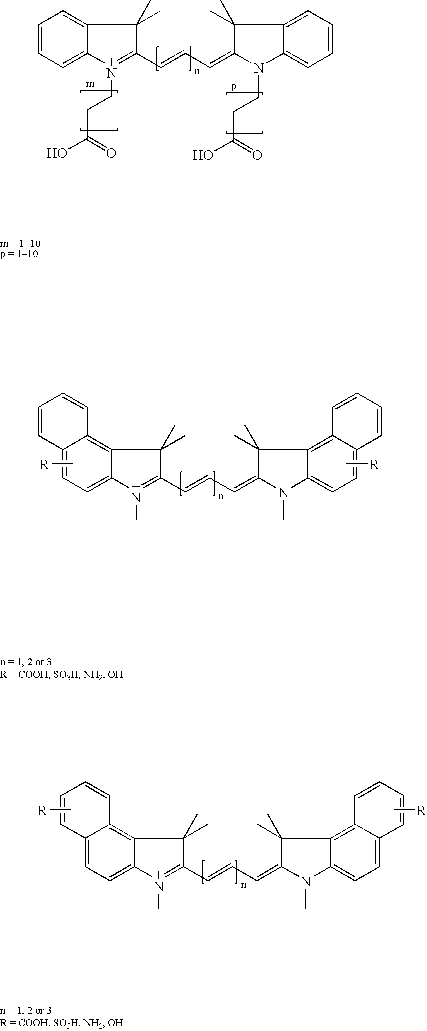

A specific example of a suitable cyanine dye derivative (denoted herein as “bis-amino Cy3”) is:

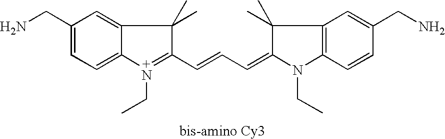

In one embodiment, exactly one, one or more, more than one, or all of the payload moieties is bis-amino Cy3 or a derivative thereof.

Payload: Payload Moieties: Specific Examples: Chelating Agents

Examples of payloads which have diagnostic value, by virtue of being, or comprising, a chelating group capable of complexing with a detectable label (e.g., a radioactive metal ion, a paramagnetic atom, etc.), include chelating agents and derivatives thereof.

In one embodiment, exactly one, one or more, more than one, or all of the payload moieties is, or comprises, a chelating group capable of complexing with a detectable label.

Examples of suitable chelating agents include poly-carboxylic acids and porphyrins, include:

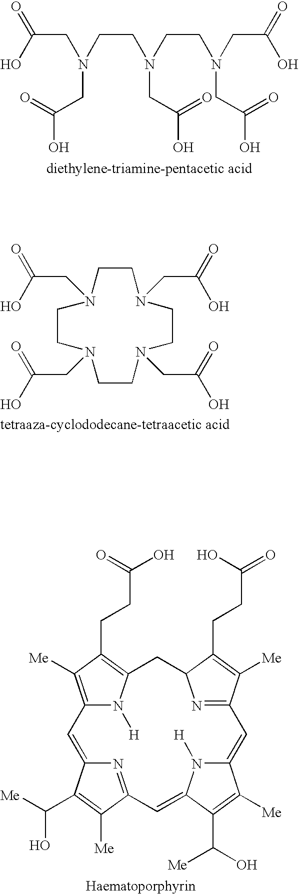

Such payloads may be incorporated into a carrier polymer via, for example, by reaction at one or more carboxylic acids groups, for example, as acid anhydrides, acid halides, esters (e.g., activated esters), and the like.

Such payloads chelate with, for example, paramagnetic ions for use as magnetic resonance imaging (MRI) contrast agents.

Payload: Payload Moieties: Specific Examples: Drugs

Examples of payloads which have therapeutic value, by virtue of being, or comprising, a drug, include compounds such as doxorubicin and fluorouracil.

In one embodiment, exactly one, one or more, more than one, or all of the payload moieties is, or comprises, a drug.

Examples of suitable drugs include:

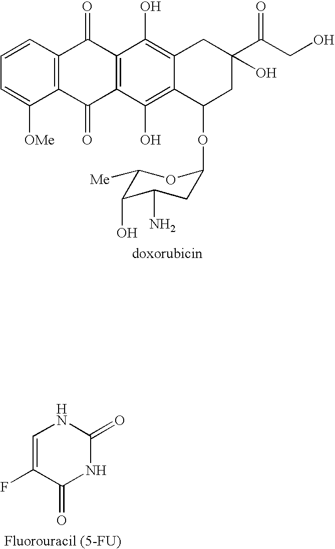

Such payloads may be incorporated into a carrier polymer via, for example, by reaction at one or more primary amino groups (—NH₂), cyclic secondary amino groups (—NH—), hydroxy groups (—OH), or ketone groups (—C(═O)R). For example, doxorubicin may be conjugated to a carrier polymer via the ketone-hydrazine reaction. See, for example, Rihova et al., 2001

Payload: Payload Moieties: Specific Examples: Boronic Acid Derivatives

In one embodiment, exactly one, one or more, more than one, or all of the payload moieties is, or comprises, a boron-containing moiety, for example, a boronic acid derivative.

Examples of suitable boronic acid derivatives include:

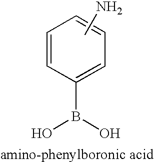

Such payloads may be incorporated into a carrier polymer via, for example, by reaction at the amino group, for example, to give an amide linkage.

Such payloads are useful, for example, as neutron capture agents, and have therapeutic applications.

Payload: Payload Moieties: Specific Examples: Peptides

In one embodiment, exactly one, one or more, more than one, or all of the payload moieties is, or comprises, a peptide.

Examples of peptides include antibodies, antibody fragments, enzymes, transcription factors, signalling proteins, antisense peptides, zinc fingers, peptide vaccines, and the like.

Examples of peptides include, but are not limited to, therapeutic peptides, for example, such as leuprolide, somatostatin and triptorelin, which are used in cancer treatment; zinc-finger transcription factors, which are used in cancer gene switching; and peptide vaccines.

Payload: Payload Moieties: “Otherwise Associated With”: Specific Examples

As discussed above, in one embodiment, the carrier polymer is otherwise associated with the payload. That is, the payload is not incorporated into the carrier polymer.

For example, when the payload is a nucleic acid (e.g., a plasmid), it may be formulated with polycationic species (e.g., polylysine) in less than molar equivalent ratios so that the overall complex possesses a net positive charge. This cationic complex is then mixed with an anionic hypercoiling carrier polymer, to yield a neutral or net anionic polyplex. In this way the hypercoiling polymer would be associated with the polyplex by electrostatic charge. (Alternatively, the cationic complex is chemically conjugated to an anionic hypercoiling carrier polymer (that is, the carrier polymer incorporates the payload), for example, via free amino groups, to yield a neutral or net anionic species.)

In one embodiment, exactly one, one or more, more than one, or all of the payload moieties is, or comprises, a nucleic acid (e.g., DNA, RNA, etc.).

In one embodiment, exactly one, one or more, more than one, or all of the payload moieties is, or comprises, a cationic nucleic acid complex.

Combinations of Features

Each compatible combination of the above features is also included as if it was explicitly recited.

Payload Delivery: Trafficking Time and Delivery Amounts

In one embodiment, the carrier polymer which incorporates the payload, or the carrier polymer and the otherwise associated payload, enters (or is capable of entering) living cells (or the nuclei of living cells) rapidly.

That is, a fraction of the carrier polymer which incorporates the payload, or the carrier polymer and the otherwise associated payload, when placed in contact with living cells, enters (or is capable of entering) the cells (or the nuclei of the cells) within an entry time which is relatively short.

In one embodiment, the fraction is a detectable fraction.

In one embodiment, the fraction is at least about 1% by weight of the carrier polymer which incorporates the payload, or the carrier polymer and the otherwise associated payload, which is placed in contact with cells.

In one embodiment, the fraction is at least about 2%; at least about 5%; at least about 10%; at least about 20%; at least about 50%.

In one embodiment, the fraction is at least about 0.01 ng of the carrier polymer which incorporates the payload, or the carrier polymer and the otherwise associated payload, per cell.

In one embodiment, the fraction is at least about 0.05 ng/cell; at least about 0.1 ng/cell; at least about 0.5 ng/cell; at least about 1 ng/cell; at least about 5 ng/cell; at least about 10 ng/cell; at least about 50 ng/cell.

The entry time is determined using conventional cell lines and conventional incubation methods.

In one embodiment, the entry time is less than about 6 hours; less than about 4 hours; less than about 3 hours; less than about 2 hours; less than about 1 hour; less than about 45 minutes; less than about 30 minutes.

Methods for determining whether or not a detectable fraction of the carrier polymer which incorporates the payload, or the carrier polymer and the otherwise associated payload, has entered the cells are well known in the art and/or are described herein.

Suitable methods include Laser Scanning Confocal Microscopy (LSCM) methods, such as those described in the Examples below (see the sections headed “Interaction of PD20 with CHO cells”).

Suitable methods are selected according to the particular carrier polymer and/or payload. For example, the carrier polymer and/or the payload may be selected to be, or to have, a detectable label, e.g., a fluorophore, which permits direct observation. In the Examples below, the payload was selected to be bis-amino Cy3, a cyanine dye; entry into cells (and the nuclei of cells) was determined and monitored using confocal microscopy.

Payload Delivery: Mechanism

In one embodiment, the payload is delivered into the living cell, or into the nucleus of a living cell, by a mechanism which involves endosomes, e.g., endosomal escape.

In one embodiment, the carrier polymer and the payload (that is, the carrier polymer which incorporates payload; or the carrier polymer and the otherwise associated payload) is capable of endosomal escape.

In one embodiment, the carrier polymer and the payload (that is, the carrier polymer which incorporates the payload, or the carrier polymer and the otherwise associated payload) is endosomolytic.

Without wishing to be bound by any particular theory, it is believed that upregulated endosomal update in rapidly proliferating cells increases the rate of delivery of the carrier polymer and the payload into such cells.

In one embodiment, the payload is delivered into the living cell, or into the nucleus of a living cell, by a mechanism which involves lipid bilayer disruption.

In one embodiment, the carrier polymer and the payload (that is, the carrier polymer which incorporates the payload, or the carrier polymer and the otherwise associated payload) is capable of lipid bilayer disruption.

In one embodiment, the carrier polymer and the payload (that is, the carrier polymer which incorporates the payload, or the carrier polymer and the otherwise associated payload) is lipid bilayer membrane-disrupting.

Without wishing to be bound by any particular theory, it is believed that ability to disrupt lipid bilayers increases the rate of delivery into the cell (or the nucleus of the cell).

Illustrative Examples

Several examples of carrier polymers, and carrier polymers incorporating payloads, are shown below.

One example of a carrier polymer is poly(lysine iso-phthalamide), shown below.

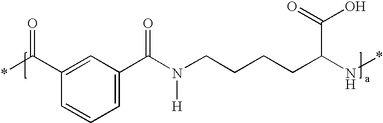

One example of a carrier polymer is hydrophobically-modified poly(β-aspartic acid) (where —NH—CHR—COOH is derived from a hydrophobic amino acid), shown below.

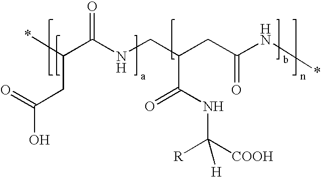

One example of a carrier polymer which incorporates (within the polymer backbone) a payload (a cyanine dye fluorophore) is poly(lysine iso-phthalamide-co-bis-amino-Cy3 iso-phthalamide), shown below.

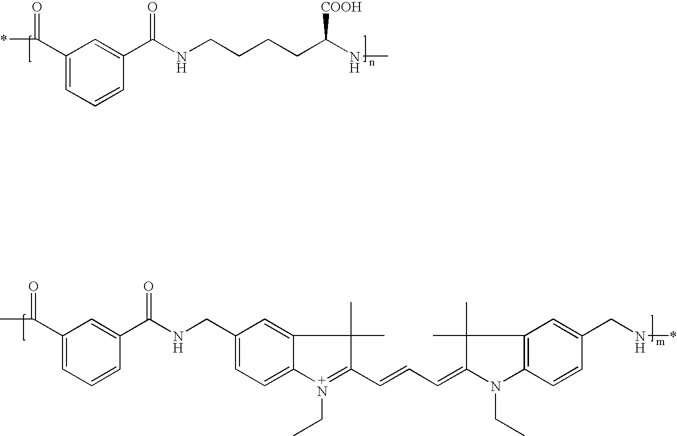

One example of a carrier polymer which incorporates (as a pendant group) a payload (a doxorubicin-derived moiety) is poly(lysine iso-phthalamide-co-lysine DOX iso-phthalamide), shown below.

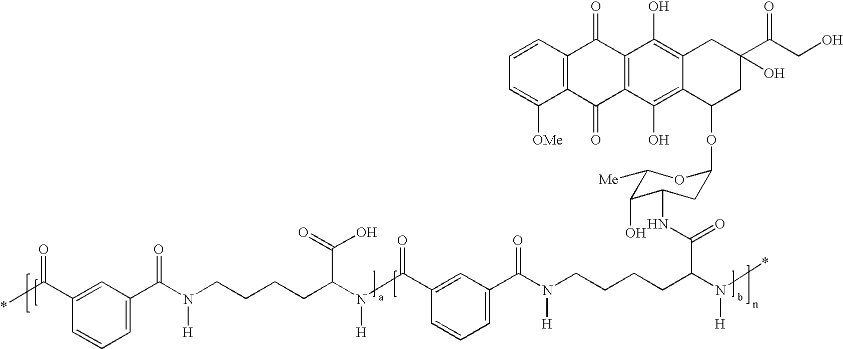

One example of a carrier polymer which incorporates (as a pendant group) a payload (a doxorubicin-derived moiety), and which is further modified to have PEG groups (where R is —O—, to give an ester linkage, or —NH— to give an amide linkage), is PEGylated poly(lysine iso-phthalamide-co-lysine DOX iso-phthalamide), shown below. 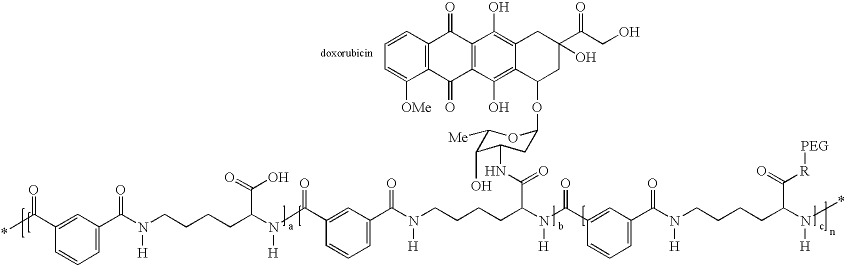
Methods of Synthesis

Several methods for the chemical synthesis of carrier polymers, which incorporate, or are otherwise associated with, a payload, are described herein. These and/or other well known methods may be modified and/or adapted in known ways in order to facilitate the synthesis of additional carrier polymer within the scope of the present invention.

As described above, in one embodiment, the polymer has a backbone having amide linkages (—C(═O)—NR—). Such linkages may be formed by reaction of various combinations of functional groups, using well known methods.

In one approach, monomers having either two amino groups (H₂N-M¹-NH₂), or two acyl halide (e.g., acyl chloride) groups (ClC(═O)M²C(═O)Cl) are employed. In this way, an alternating polymer may be formed. An example of this approach is illustrated in the following scheme.

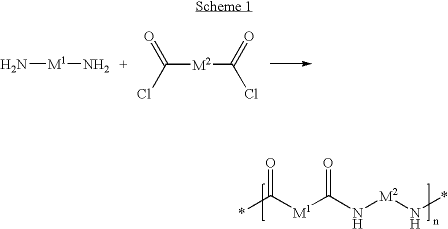

The groups M¹ and M² may be selected to yield the desired polymer. For example, M¹ may be selected for its hydrophobic properties, and M² may be selected for its hydrophilic properties.

For example, a first monomer may be selected to be lysine (R═H) (or a lysine ester, e.g., R=Et) (to provide-hydrophilic moieties) and a second monomer (ClC(═O)M²C(═O)Cl) may be selected to be iso-phthaloyl chloride (to provide hydrophobic moieties). The resulting polymer is poly(lysine iso-phthalamide), as illustrated in the following scheme.

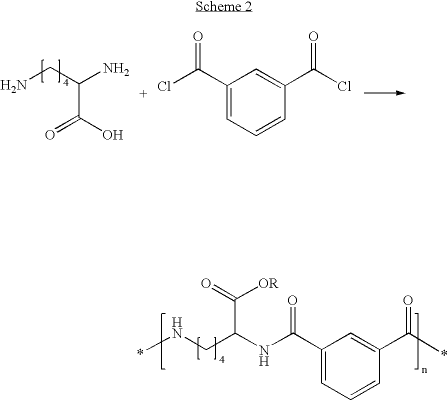

If it is desired to synthesize a carrier polymer which incorporates the payload, then the synthesis method may be adapted accordingly. For example, the payload may be selected to have, or the payload may be derivatized to have, suitable reactive functional groups.

In one approach, monomers having either two amino groups (H₂N-M¹-NH₂), or two acyl halide (e.g., acyl chloride) groups (ClC(═O)M²C(═O)Cl) are employed, along with a payload also having two amino groups (H₂N-PL-NH₂). In this way, an alternating polymer may be formed. An example of this approach is illustrated in the following scheme.

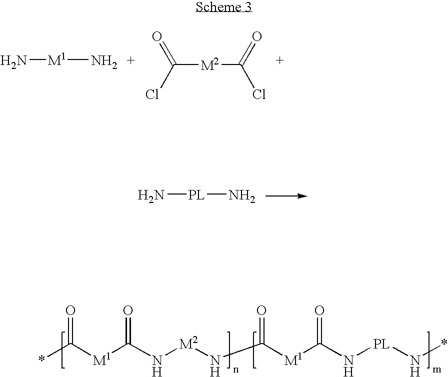

For example, a first monomer may be selected to be lysine (R═H) (or a lysine ester, e.g., R=Et) (to provide hydrophilic moieties), a second monomer (ClC(═O)M²C(═O)Cl) may be selected to be iso-phthaloyl chloride (to provide hydrophobic moieties), and the payload may be selected to be bis-amino Cy3 (denoted here as NH₂CH₂-Z-CH₂NH₂), a cyanine dye having two amino groups. The resulting polymer is poly(lysine iso-phthalamide)-copoly(lysine-bis-amino-Cy3), as illustrated in the following scheme.

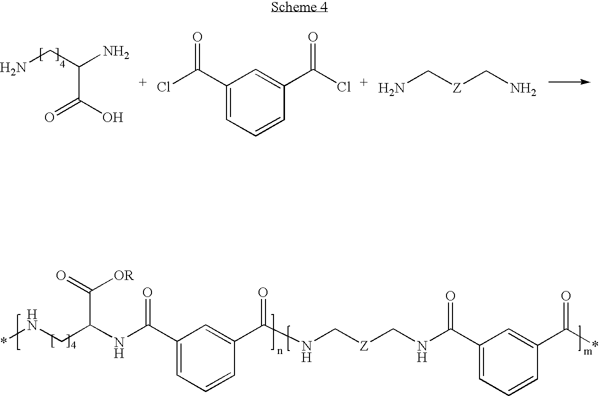

In another approach, polyesters may be prepared, for example, from benzyl malolactonate, having pendant carboxylic acid groups, which can be further reacted with, for example an amino compound, for example, an amino acid, bearing a hydrophobic group, to form pendant amides groups: An example of this approach is illustrated in the following scheme. Examples of suitable amino acids, R—CH(NH₂)COOH include, but are not limited to, alanine, valine, norvaline, leucine, isoleucine, norleucine, phenylalanine, phenylglycine, tyrosine, tryptophan.

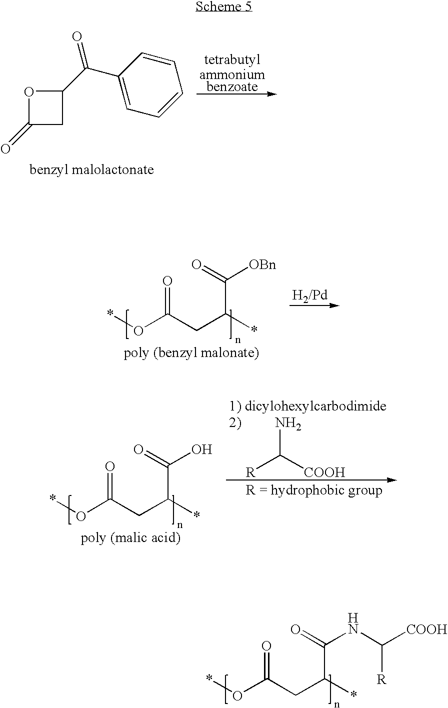

In another approach, polyamides may be prepared, for example, from aspartic acid, having pendant carboxylic acid groups, some or all of which can be further reacted with, for example an amino compound, for example, an amino acid, bearing a hydrophobic group, to form pendant amides groups. An example of this approach is illustrated in the following scheme. Examples of suitable amino acids, R—CH(NH₂)COOH include, but are not limited to, alanine, valine, norvaline, leucine, isoleucine, norleucine, phenylalanine, phenylglycine, tyrosine, tryptophan. 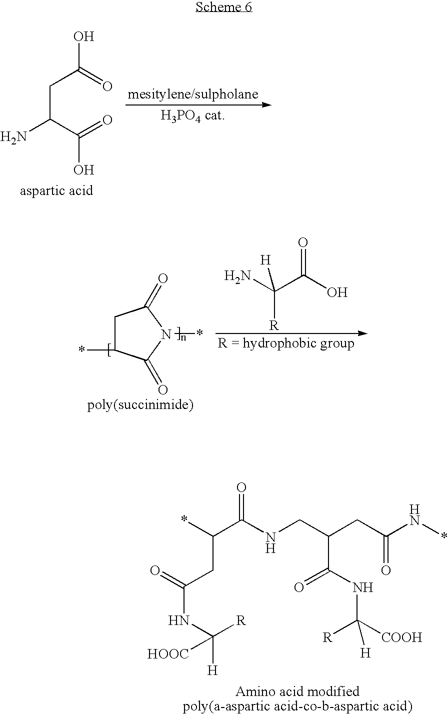
Uses: Methods of Delivery

As described above, the present invention pertains to methods of delivery payloads into a living cell, or into the nucleus of a living cell.

Delivery of a payload into a living cell, or into the nucleus of a living cell may form part of another method, for example, a therapeutic method, a diagnostic method, a method of imaging, etc.

Uses: Therapeutic Uses

One aspect of the present invention pertains to a hypercoiling carrier polymer which incorporates a payload, or which is otherwise associated with a payload, as described herein, for use in a method of treatment of the human or animal body by therapy.

One aspect of the present invention pertains to use of a hypercoiling carrier polymer which incorporates a payload, or which is otherwise associated with a payload, as described herein, for the preparation of a medicament for the treatment of a condition (e.g., a pathology) which is treatable by said payload.

One aspect of the present invention pertains to a method of treatment of a condition (e.g., a pathology) comprising administering to a patient suffering from said condition a therapeutically-effective amount of a hypercoiling carrier polymer which incorporates a payload, or which is otherwise associated with a payload, as described herein, wherein said payload is a drug which treats said condition.

Uses: Diagnostic Uses

One aspect of the present invention pertains to a hypercoiling carrier polymer which incorporates a payload, or which is otherwise associated with a payload, as described herein, for use in a method of diagnosis practiced on the human or animal body.

One aspect of the present invention pertains to a method of diagnosis of a condition (e.g., a pathology) comprising:

(a) administering to a patient an effective amount of a hypercoiling carrier polymer which incorporates a payload, or which is otherwise associated with a payload, as described herein, wherein said payload is, or comprises, a detectable label;

(b) detecting the presence and/or location of said detectable label; and

(c) correlating said presence and/or location with said condition.

The phrase “correlating with said condition” includes, for example, correlating with the presence, absence, degree, progress, amelioration, regression, cure, etc., of the condition.

Uses: Methods of Imaging

One aspect of the present invention pertains to a method of imaging a cell comprising:

(a) contacting a living cell with a hypercoiling carrier polymer which incorporates a payload, or which is otherwise associated with a payload, as described herein, further wherein said payload is, or comprises, a detectable label; and

(b) detecting the presence and/or location of said detectable label.

In one embodiment, the method further comprises the step of:

(c) forming an image of said cell using said presence and/or location data.

One aspect of the present invention pertains to a method of imaging a patient, or a portion thereof, comprising:

(a) administering to said patient an effective amount of a hypercoiling carrier polymer which incorporates a payload, or which is otherwise associated with a payload, as described herein, further wherein said payload is, or comprises, a detectable label; and

(b) detecting the presence and/or location of said detectable label;

In one embodiment, the method further comprises the step of:

(c) forming an image of said patient, or portion thereof using said presence and/or location data.

In one embodiment, the image is a 2-dimensional image.

In one embodiment, the image is a 3-dimensional image.

In one embodiment, there is a period of time (e.g., a waiting time) between steps (a) and (b). The waiting time may be, for example, 1 minute, 10 minutes, 30 minutes, 1 hour, 2 hours, 6 hours, 12 hours, 1 day, 2 days, etc.

In one embodiment, step (b) is repeated at (e.g., regular) time intervals.

One aspect of the present invention pertains to a method of imaging, as described herein, at exactly two, two or more, or more than two, times. For example, a plurality of images may be obtained, for example, at intervals of 1 second, 10 seconds, 30 seconds, 1 minute, 5 minutes, 15 minutes, 30 minutes, 1 hour, 2 hours, 6 hours, etc. Such methods may be used to generate dynamic “motion pictures” or “movies” based upon the images, for example, to show the progress of cell (or nuclear) delivery over time.

Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis) is also included.

The term “therapeutically-effective amount,” as used herein, pertains to that amount of an active compound, or a material, composition or dosage form comprising an active compound, which is effective for producing some desired therapeutic effect, commensurate with a reasonable benefit/risk ratio.

The term “treatment” includes combination treatments and therapies, in which two or more treatments or therapies are combined, for example, sequentially or simultaneously. Examples of treatments and therapies include, but are not limited to, chemotherapy (the administration of active agents, including, e.g., drugs, antibodies (e.g., as in immunotherapy), prodrugs (e.g., as in photodynamic therapy, GDEPT, ADEPT, etc.); surgery; radiation therapy; and gene therapy.

Cells

The cell, that is, the living cell into which the payload is delivered, may be of any type.

For example, the cell may prokaryotic (e.g., bacteria) or eukaryotic (e.g., protoctista, fungi, plants, animals).

For example, the cell may be a cell of, or derived from, a patient, as described below. Such a cell may be, for example, in situ (e.g., in vivo), or may be removed from its source (e.g., ex vivo, cultured, etc.). Thus, the methods described herein may be performed, e.g., in vivo or ex vivo.

Patients

The patient may be an animal, a chordate, a vertebrate, a mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human. Furthermore, the patient may be any of its forms of development, for example, an egg, a foetus.

Routes of Administration

Administration may be by any convenient route, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

Dosage

It will be appreciated by one of skill in the art that appropriate dosages can vary from patient to patient. Determining the optimal dosage will generally involve the balancing of the level of therapeutic/diagnostic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy/diagnosis, the purpose of the therapy/diagnosis, the target cell(s) being treated, and the subject being treated. Single or multiple administrations can be carried out with the dose level and pattern being selected by the treating physician, veterinarian, or clinician.

In general, a suitable dose of the active compound is in the range of about 100 μg to about 250 mg per kilogram body weight of the subject per day.

Kits

One aspect of the invention pertains to a kit comprising (a) a carrier polymer, or a carrier polymer and a payload, as described herein, preferably provided in a suitable container and/or with suitable packaging; and (b) instructions for use, for example, written instructions on how to perform methods of diagnosis, imaging, treatment, etc., as described above.

Optionally, the kit may further comprise appropriate reagents (e.g., buffers, solvents) and/or devices (e.g., tubes, syringes) for assembly and/or use (e.g., administration).

### EXAMPLES

The following are examples are provided solely to illustrate the present invention and are not intended to limit the scope of the invention, as described herein.

### Example 1

### Naked Polymer: Poly(L-lysine iso-phthlamide)

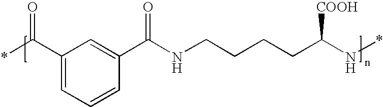

L-Lysine (14.621 g, MW 146.21, 100 mM) (Aldrich chemical company. Gillingham, Dorset, UK) and potassium carbonate (41.463 g, MW 138.21, 300 mM) were dissolved in 500 mL deionised water and cooled to 0° C. iso-phthaloyl chloride (20.3 g, 203.02, 100 mM) (Aldrich) was dissolved in 500 mL acetone, pre-cooled to 0° C. The organic solution was added to aqueous phase stirred in a 2 litre Waring commercial laboratory blender (Fisher Scientific, Loughborough, Leicestershire, UK) at full speed, and stirring was maintained for 30 minutes.

The acetone was then removed under vacuum at 30° C. The aqueous phase was dialysed using a vivaflow50™ ultrafiltration unit (Vivascience, Hannover, Germany) containing a poly(ether sulphone) diafiltration membrane with a molecular weight cut-off of 5 kDa with 2 litres of deionised water to remove inorganic salts, low molecular weight oligomers, and residual organic solvent.

The resulting solution was then lyophilised in a Virtis Advantage freeze drier (Virtis, 815 Route 208, Gardiner, N.Y., USA) to give 11 g of a white solid, which was stored at 4° C.

### Example 2

### Naked Polymer: Poly(L-lysine iso-phthlamide)

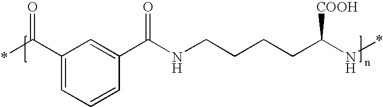

L-Lysine ethyl ester dihydrochloride (24.714 g, MW 247.14, 100 mM) (Aldrich) and potassium carbonate (55.284 g, 138.21, 400 mM) were dissolved in 500 mL deionised water and cooled to 0° C. iso-Phthaloyl chloride (20.3 g, 203.02, 100 mM) (Aldrich) was dissolved in 500 mL acetone, pre-cooled to 0° C. The organic solution was added to the rapidly stirred aqueous phase (Waring blender, full speed), and stirring was maintained for 30 minutes.

The resulting poly(L-lysine ethyl ester iso-phthlamide) was removed and washed with 200 mL deionized water, and then dissolved in 200 mL of dimethyl sulphoxide (DMSO) in a one litre beaker. To this solution, a solution of 20 g sodium hydroxide dissolved in 400 mL absolute ethanol was added over 5 minutes while stirring with a carousel hotplate (Radleys Discovery Technologies Ltd, Saffron Walden, Essex, UK). The poly(L-lysine iso-phthlamide) thus formed was removed by vacuum filtration, then stirred in suspension in 200 mL hot absolute alcohol for 30 minutes, and then re-filtered. The ethanol rinse step was repeated four times, and then the polymer (20 g) was dried in a vacuum oven at 40° C. overnight, and stored at 4° C.

### Example 3

### Polydye 20: Poly(L-lysine-co-Cy3-iso-phthalamide)

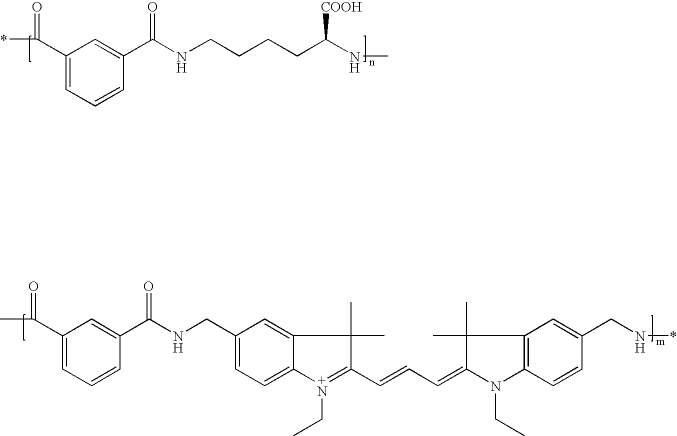

Fluorophore labelled polymers (“Polydyes” or PD's hereafter) were prepared by copolymerising L-lysine and a bis-amine Cy3 cyanine fluorophore derivative with iso-phthaloyl chloride. The polymerisation technique was similar to that for the naked polymer, poly(L-lysine iso-phthlamide).

L-Lysine (1.462 g, MW 146.21, 10 mM) (Aldrich), bis-amino Cy3 (0.21 g, MW 558, 0.5 mM) (Nycomed Amersham, Cardiff, Wales), and potassium carbonate (4.29 g, MW 138.21, 31 mM) were dissolved in 50 mL deionised water and cooled to 0° C. iso-Phthaloyl chloride (2.132 g, MW 203.02, 10.5 mM) (Aldrich) was dissolved in 50 mL acetone, pre-cooled to 0° C. The organic solution was added to the rapidly stirred aqueous phase (Waring blender, full speed), and stirring was maintained for 30 minutes.

The acetone was then removed under vacuum. The aqueous phase was dialysed using a vivaflow50™ ultrafiltration unit (Vivascience, Hannover, Germany) containing a poly(ether sulphone) diafiltration membrane with a molecular weight cut-off of 5 kDa with 200 mL of deionised water to remove inorganic salts, low molecular weight oligomers, and residual organic solvent.

The resulting solution was then lyophilised in a Virtis AdVantage freeze drier (Virtis, 815 Route 208, Gardiner, N.Y., USA) to give 1.36 g of a deeply purple solid, which was stored at 4° C.

### Examples 4, 5, 6, 7, 8

### Polydyes: Poly(L-lysine-co-Cy3-iso-phthalamide)

Polymers with higher molar ratios of L-Lysine to bis-amino Cy3 were prepared by similar methods. The Polydye number (e.g., PD20) denotes the molar ratio of L-lysine to bis-amino Cy3 in the reaction mixture.

| | | Reagents (mM) | | |
|---|---|---|---|---|
| Ex# | Polymer | L-Lysine | bis-amino Cy3 | iso-Phthaloyl chloride |
| 4 | PD20 | 10 | 0.500 | 10.500 |
| 5 | PD30 | 10 | 0.333 | 10.333 |
| 6 | PD40 | 10 | 0.250 | 10.250 |
| 7 | PD60 | 10 | 0.166 | 10.166 |
| 8 | PD80 | 10 | 0.125 | 10.125 |

### Example 9

### Poly(L-lysine-co-L-lysine ethyl ester iso-phthalamide)

L-Lysine (11.697 g, MW 146.21, 80 mM) (Aldrich Chemical Company, Gillingham, Dorset, UK), L-lysine ethyl ester dihydrochloride (4.943 g, MW 247.14, 20 mM) and potassium carbonate (44.227 g, MW 138.21, 320 mM) were dissolved in 500 mL deionised water and cooled to 0° C. iso-Phthaloyl chloride (20.3 g, 203.02, 100 mM) (Aldrich Chemical Company) was dissolved in 500 mL acetone, pre-cooled to 0° C. The organic solution was added to aqueous phase stirred in a 2 litre Waring commercial laboratory blender (Fisher Scientific, Loughborough, Leicestershire, UK) at full speed, and stirring was maintained for 30 minutes.

The acetone was then removed under vacuum at 30° C. The aqueous phase was dialysed using a Vivaflow50™ ultrafiltration unit (Vivascience, Hannover, Germany) containing a poly(ether sulphone) diafiltration membrane with a molecular weight cut-off of 5 kDa with 2 litres of deionised water to remove inorganic salts, low molecular weight oligomers, and residual organic solvent.

The resulting solution was then lyophilised in a Virtis Advantage freeze drier (Virtis, 815 Route 208, Gardiner, N.Y., USA) to give 10.5 g of a white solid, which was stored at 4° C.

### Example 10

### Poly(L-lysine dodecanamide)

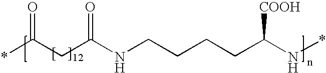

L-Lysine monohydrochloride (18.265 g, MW 182.65, 100 mM) (Aldrich Chemical Company, Gillingham, Dorset, UK) and potassium carbonate (110.568 g, MW 138.21, 800 mM) were dissolved in 500 mL deionised water and cooled to 0° C. Dodecandioyldichloride (26.7 g, MW 267.20, 100 mM) (Aldrich Chemical Company) was dissolved in 500 mL chloroform, pre-cooled to 0° C. The organic solution was added to aqueous phase stirred in a 2 litre Waring commercial laboratory blender (Fisher Scientific, Loughborough, Leicestershire, UK) at full speed, and stirring was maintained for 30 minutes.

The aqueous phase was separated for the organic phase in a separating funnel and dialysed using a Vivaflow50™ ultrafiltration unit (Vivascience, Hannover, Germany) containing a poly(ether sulphone) diafiltration membrane with a molecular weight cut-off of 5 kDa with 2 litres of deionised water to remove inorganic salts, low molecular weight oligomers, and residual organic solvent.

The resulting solution was then lyophilised in a Virtis Advantage freeze drier (Virtis, 815 Route 208, Gardiner, N.Y., USA) to give 18.5 g of a white solid, which was stored at 4° C.

### Example 11

### Poly(L-lysine butylmalonamide)

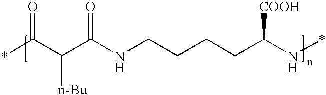

Butylmalonyl chloride (19.72 g, MW 197.17, 100 mmol) (Aldrich Chemical Company) was generated in-situ by the dropwise addition of oxaloyl chloride (27 mL, MW 126.93, 300 mmol) to butylmalonic acid (16.02 g, MW 160.17, 100 mmol) dissolved in 250 mL anhydrous chloroform with a drop of dimethylformamide (DMF), pre-cooled to 0° C. After two hours, the solvent was removed on a rotary evaporator at 45° C. followed by heating to 65° C. to remove unreacted oxaloyl chloride. The remaining yellow oil was dissolved in 500 mL anhydrous chloroform. L-Lysine ethyl ester dihydrochloride (24.714 g, MW 247.14, 100 mmol) (Aldrich Chemical Company, Gillingham, Dorset, UK) and potassium carbonate (110.568 g, MW 138.21, 800 mmol) were dissolved in 500 mL deionised water and cooled to 0° C. The organic phase was added to aqueous phase stirred in a 2 litre Waring commercial laboratory blender (Fisher Scientific, Loughborough, Leicestershire, UK) at full speed, and stirring was maintained for 30 minutes.

The polymer was recovered as a gummy precipitate at the end of the reaction and washed with 200 mL deionised water and dissolved in 200 mL of dimethyl sulphoxide (DMSO) in a one litre beaker. To this solution, a solution of 20 g sodium hydroxide dissolved in 400 mL absolute ethanol was added over 5 minutes while stirring with a carousel hotplate (Radleys Discovery Technologies Ltd, Saffron Walden, Essex, UK). The poly(L-lysine butylmalonamide) thus formed was removed by vacuum filtration, then stirred in suspension in 200 mL hot absolute alcohol for 30 minutes, and then re-filtered. The ethanol rinse step was repeated four times, and then the polymer (18 g) was dried in a vacuum oven at 40° C. overnight, and stored at 4° C.

The aqueous phase was separated for the organic phase in a separating funnel and dialysed using a Vivaflow50™ ultrafiltration unit (Vivascience, Hannover, Germany) containing a poly(ether sulphone) diafiltration membrane with a molecular weight cut-off of 5 kDa with 2 litres of deionised water to remove inorganic salts, low molecular weight oligomers, and residual organic solvent.

The resulting solution was then lyophilised in a Virtis Advantage freeze drier (Virtis, 815 Route 208, Gardiner, N.Y., USA) to give 18.5 g of a white solid, which was stored at 4° C.

IR Analysis

FT-IR analysis (Nicolet 510 FT-IR spectrometer, Thermo Electron Spectroscopy, Cambridge, UK) of the naked polymer precipitates showed characteristic absorptions due to the carboxylic acid C═O stretch (˜1,710 cm⁻¹) with strong absorption at ˜1,640 cm⁻¹ (amide band I) and at ˜1,542 cm⁻¹ (amide band II).

FT-IR analysis of the polydyes (PDs) showed absorptions identical to those of the naked polymer.

NMR Analysis

¹H NMR analysis (Bruker 300 MHz, Bruker BioSpin Limited, Coventry, West Midlands, UK) of the polydye samples was attempted to determine the dye loading and distribution of fluorophores within the polymer backbone. However, spectral broadening due to sample viscosity, coupled with the low levels of dye incorporation, degraded resolution such that the determination of fluorophore to polymer ratios and the relative distribution of the fluorophores was not possible.

¹H and ¹³C NMR analysis was used to confirm the pseudo-polypeptide structure of poly(L-lysine iso-phthalamide) and poly(L-lysine ethyl ester iso-phthalamide).

¹H and ¹³C NMR analysis was also used to confirm the complete hydrolysis of the ethyl ester groups of poly(L-lysine ethyl ester iso-phthalamide) by treatment of the DMSO solution of the polymer with ethanolic sodium hydroxide, as described above.

Gel Permeation Chromatography (GPC)

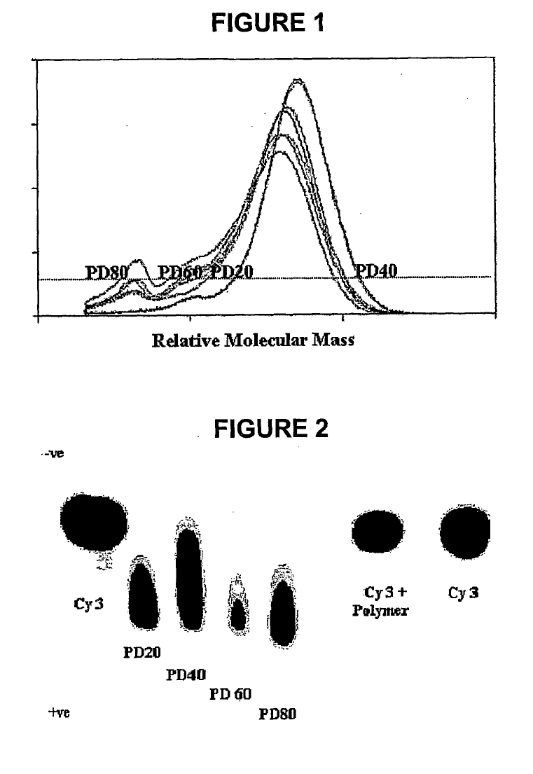

The lyophilised polycarboxylate salts were insoluble in dimethylformamide (DMF) and so aqueous gel permeation chromatograms were obtained for samples of PD20, PD40, PD60, and PD80 using two 30 cm Viscotek GMPW columns and a 0.2 M sodium nitrate, 0.01 M sodium dihydrogenphosphate buffer adjusted to pH 7.0 as eluent. The chromatograms are shown in FIG. 1.

Since the rate of Cy3 bis-amino monomer incorporation into the polymer might be different from that of lysine, the possibility exists that the relative molecular weights of the fluorophore-incorporated copolymers might vary with fluorophore loading. Furthermore, the distribution of fluorophores in the polymers might be non-random, with local incorporation of multiple fluorophores leading to fluorescence quenching even in the extended polymer chains. To reduce the probability of such effects extremely low levels of fluorophore incorporation were used such that the maximum mean level of dye incorporation in the fluorescent copolymers reported here corresponded to just one bis-amino Cy3 monomer per 20.6 kDa of polymer (PD20).

In order to examine whether the relative molecular weights of fluorophore-incorporated copolymers vary with degree of fluorophore loading, gel permeation chromatograms were obtained, as described above. With the exception of PD40, which is of slightly higher mean molecular weight of the high degree of superposition of gel permeation chromatograms for each of the polydye samples indicates little difference in relative molecular mass or polydispersity for the different fluorophore loadings.

Under these conditions, the relative molecular weights in aqueous solution expressed as PEG/PEO equivalents were apparently low (<6000 Da) and their validity was questioned since solvation of the charged Polydyes at pH 7.0 would be expected to differ considerably from that of the PEG/PEO calibrants, resulting in different hydrodynamic radii for equivalent absolute molecular weights (see, e.g., Strying et al., 1989). All of the Polydye samples chromatographed similarly with the exception of the Polydye 40 sample, which showed a slightly higher retention time. See FIG. 1. Relatively low equivalent molecular weights were obtained possibly due to peak broadening caused by retention of material on the columns and structural differences between the analysed samples and the calibrants.

To investigate this further, the Polydye 40 sample was also analysed by a commercial analyst (Viscotek Europe, Chapel House, Kingsclere Road, Basingstoke) under similar conditions and also using 0.1 M sodium nitrate/15% methanol as eluent. A triple detector system employing low angle light scattering, refractive index and viscometry detection was used to obtain as much structural information on the polymer as possible. With this system the light scattering detector can be used to calculate the polymer molecular weight rather than relying on relative values derived from standards. When 0.1 M sodium nitrate/0.01 M sodium dihydrogen phosphate was used as the eluent, tailing was observed using the light scattering detector. Light scattering detectors have increased sensitivity towards high molecular weight compounds, and a late eluting tail is characteristic of bound high molecular weight material eluting from the column. When the eluent was replaced with 0.1 M sodium nitrate/15% methanol, the elution profile of the injected material was much improved. The molecular weight calculated from the light scattering data is shown in Table 1. The sample was run twice at a concentration of 6.14 mg/mL with excellent reproducibility. A Mark-Houwink “a” value, indicative of chain stiffness, of 1.15 was obtained. This compares to a value of 0.7 for polystyrene in THF, a classic random coil, indicating increased chain stiffness, a result of the electrostatic repulsion along the polymer backbone.

TABLE 1

| Sam- | MW | MN | MZ | | IV | Conc. | DN/DC |
|---|---|---|---|---|---|---|---|
| ple | (D) | (D) | (D) | PD | (DL/g) | (mg/mL) | (mg/g) |
| P40 | 100900 | 89360 | 118800 | 1.13 | 0.110 | 6.14 | 0.183 |
| P40 | 103000 | 90260 | 123000 | 1.14 | 0.110 | 6.14 | 0.183 |

Gel Filtration (GF)

Gel filtration analysis was conducted to confirm the conjugation of the fluorophore within the poly(L-lysine iso-phthalamide) backbone.

Specifically, the elution behaviour of PD20 and unconjugated bis-amino Cy3 from a Superdex-75 gel permeation column were separately studied.

For the gel filtration analyses, 1 litre of 0.1 M phosphate buffered saline was run through a Superdex 75 column (Hiload 16/60, Amersham Biosciences UK Limited, Little Chalfont, Buckinghamshire, UK). 1 mL of a 1 mg/mL solution of PD20 in 0.1 M PBS was loaded onto the column and then eluted with 0.1 M phosphate buffered saline at a flow rate of 1 mL/min. Samples were collected at 2 minute intervals and their fluorescence was measured (Cytofluor™ plate reader, λₑₓ ˜530/25 nm, λₑₘ ˜590/20 nm). This procedure was repeated for unpolymerised bis-amino Cy3 and fluorescence measurements were taken in the same manner.

It was observed that bis-amino Cy3 eluted at approximately 100 mL (fraction 50) whilst PD20 eluted in a broad band from 30 to 90 mL (fraction 15 to 45) with no peak evident at 100 mL (fraction 50) (data not shown).

Poly Acrylamide Gel Electrophoresis (PAGE)

Poly acrylamide gel electrophoresis (PAGE) was performed on each of unpolymerised bis-amino Cy3; PD20; PD40; PD60; PD80; and a mixture of poly(lysine iso-phthalamide) and bis-amino Cy3; using a Mini-PROTEAN II gel electrophoresis kit with a 12% Bio-Rad Tris-HCl pre-cast polyacrylamide gel (Bio-Rad Laboratories Ltd., Hemel Hempstead, Hertfordshire, UK). Prior to each run, the sample wells were washed first with distilled water followed by the running buffer (Tris/Tricine). 10 μL samples of each of the polydyes (1 g/l in deionized water) were mixed with an equal volume of loading buffer (40% aqueous sucrose) and loaded into the wells of the gel. Bis-amino Cy3 and a mixture of the free bis-amino Cy3 with poly(L-lysine iso-phthalamide) were run as controls. The gel was then developed at 200 V in a Tris/Tricine buffer (1.0 M Tris.HCl) at pH 7.4. The gels were imaged using a LEADseeker™ (Nycomed-Amersham, Cardiff, Wales) high definition fluorescence CCD image acquisition unit (λₑₓ 535 nm, λₑₘ 595 nm, 10 nm band widths on excitation filter).

A fluorescence image of the gel, indicating the relative electrophoretic mobilities, is shown in FIG. 2.

All the Polydye samples displayed smeared bands but a clear distinction was made between these samples, that moved to the anode, and the bis-amino Cy3 controls that moved to the cathode both in the presence and absence of poly(lysine iso-phthalamide). No evidence of association of free bis-amino Cy3 to poly(lysine iso-phthalamide) was observed in the mixed sample during electrophoresis. The electrophoresis bands for PD20, PD60 and PD80 were similar, as anticipated from the GPC data and there was no evidence of residual non-polymerised bis-amino Cy3 in these samples. For PD40, the longer tail indicates a broader range of molecular weight that may arise from incomplete dialysis.

Excitation and Emission Spectra

Excitation and emission spectra of all of the polydyes were similar to those of the unpolymerised bis-amino Cy3, although the absorption and emission maxima were shifted slightly to higher wavelengths (λₑₓ from 550 nm to 553 nm, λₑₘ from 570 nm to 574 nm), consistent with their reduced mobility within the polymer backbone.

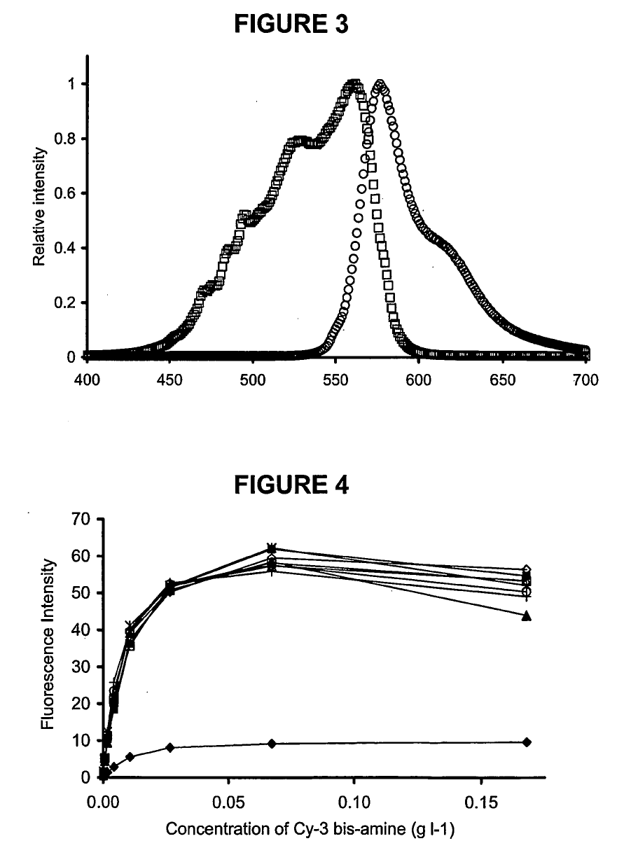

Normalised excitation and emission spectra for PD20 measured in aqueous solution (0.01 mg/mL using an Aminco luminescence spectrofluorimeter (λₑₓ 540 nm, λₑₘ 595 nm) are shown in FIG. 3.

Absorption Spectroscopy

Absorption spectroscopy was used to determine the fluorophore content of the polydye samples. Beer-Lambert plots were determined for each of the polydye samples at 553 nm using a Perkin-Elmer UV-Vis spectrophotometer (Perkin Elmer Instruments, Beaconsfield, Buckinghamshire, UK). The fluorescence output of the labelled polymers was determined using either a Cytofluor™ 4000 fluorescent multi well plate reader (λₑₓ 530/25 nm λₑₘ 580/50 nm) (Applied Biosystems, Cheshire, UK) or an Aminco SPF-125 spectrofluorimeter (λₑₓ 550 nm λₑₘ 575 nm) (Thermo Spectronic, Rochester, N.Y., United States). The fluorimeter was equipped with a flow-cell for spectrophotometric titrations and 50 mL solutions of the fluorescent probe or solutions of the base polymer in the presence of free fluorophore were titrated with 1.0 M HCl using a Radiometer TIM 900 autotitrator (Radiometer Limited, Crawley, West Sussex, UK) equipped with a 5 mL burette. The polydye solution from the titration vessel was circulated through the flow cell using a Masterflex 7518-00 peristaltic pump (Cole-Palmer Instrument company, Bishop's Stortford, Herfordshire, UK).

Relative Fluorescence Intensity

In an effort to establish the interaction between naked polymer (poly(lysine iso-phthalamide)) and bis-amino Cy3 and its influence upon fluorescence, the relative fluorescence intensity of a range of concentrations of bis-amino Cy3 (0-0.168 g/L) with different concentrations of poly(lysine iso-phthalamide), was measured using a Cytofluor plate reader (λₑₓ 535 nm and λₑₘ 570 nm) in PBS at pH 7.4. The data are shown in FIG. 4.

Under such conditions the cationic amino groups interact electrostatically with the anionic carboxyl groups. The relative fluorescence intensity of the bis-amino Cy3 was enhanced upon binding to the polymer, presumably due to the reduced mobility of the bound fluorophore (see, e.g., Reddington, 1998). Such fluorescence was strongly dependent upon the bis-amino Cy3 concentration and varied little above a critical polymer concentration.

Relative Fluorescence as a Function of Concentration

The concentration dependence of the relative fluorescence intensities of each of the polydyes was determined in phosphate buffer at pH 7.0.

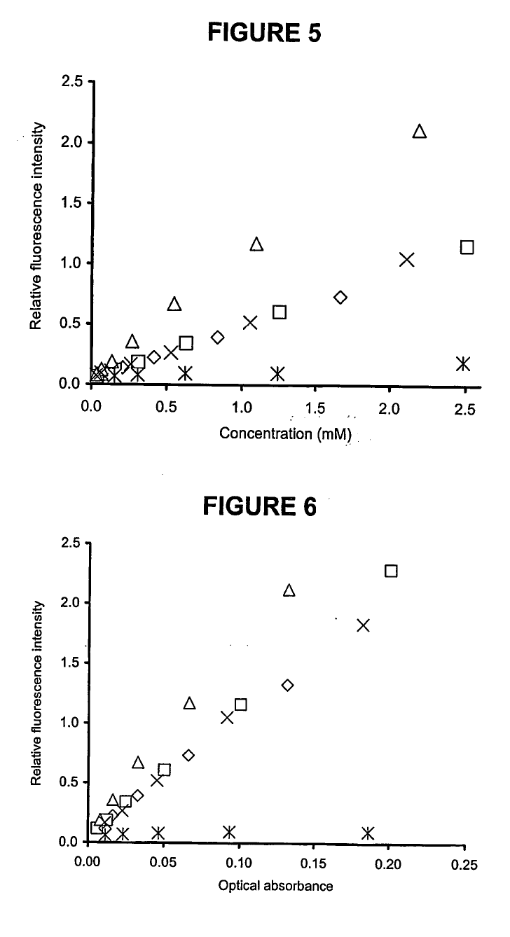

First, the concentration of single fluorophore-containing polymer segments was estimated from the relative concentrations of monomers (L-lysine and bis-amino Cy3) in the reaction mixture. The data are shown in FIG. 5.

On this basis there was little difference in fluorescence between PD20, PD60, and PD80, though PD40 showed a higher fluorescence output. However, in view of the marked difference in chemical structure and size of the L-lysine and bis-amino Cy3 monomers the implicit assumption of stoichiometric incorporation of each monomer into the polymer may well be invalid.

Second, the fluorophore contents of the probes were also estimated from Beer-Lambert calibrations using a constant extinction coefficient of 150 000 l mol⁻¹ cm⁻¹ for the polymerised fluorophore, the same as that for the free Cy3 fluorophore (see, e.g., Ballou et al., 1997). The Beer-Lambert plots for each probe were expressed as absorption verses concentration, from which the average molecular weight of a fluorophore-containing polymer segment was estimated. These average molecular weights are compared with those estimated for stoichiometric fluorophore incorporation in Table 2.

Shown in Table 2 is a comparison of average molecular weights of fluorophore containing polymer segments and average relative ratios of fluorophores in polydyes; (a) calculated on the basis of stoichiometric incorporation of bis-amino Cy3 in Polydyes and (b) calculated from an extinction co-efficient of the polymerised fluorophores equivalent to that of unmodified Cy3 in aqueous solution (150,000 L mol⁻¹ cm⁻¹).

TABLE 2

| | Relative molecular | | | |
|---|---|---|---|---|
| | mass of fluorophore | | Relative ratio | |
| | containing Polydye segments | | of Fluorophores | |
| | (a) assuming | (b) inferred | (a) assuming | (b) inferred |
| | stoichiometric | from UV | stoichiometric | from UV |
| | incorporation of | absorption | incorporation of | absorption |
| Polydye | bis-amino Cy3 | measurements | bis-amino Cy3 | measurements |
| 20 | 6 069 | 20 589 | 3.73 | 4.15 |
| 40 | 11 591 | 28 380 | 1.95 | 3.01 |
| 60 | 17 116 | 63 597 | 1.32 | 1.34 |
| 80 | 22 640 | 85 499 | 1.0 | 1.0 |

The results suggest that in each case the relative amount of fluorophore incorporated in the final polymer was lower than that initially present in the reaction mixture.

With the exception of PD40, which apparently contains relatively more fluorophores per polymer molecule than the other Polydyes, the ratio of molecular weights (based on extinction coefficients) in the Polydye series (20:40:60:80) correlate well with those calculated from the concentrations of fluorophores in the initial reaction mixtures.

The fluorescence efficiency of fluorophores is known to change with their environment and may be reduced by fluorescence quenching at high concentrations. The possibility exists that non-random bis-amino Cy3 incorporation might result in block copolymer formation with high local concentrations of dye at certain regions of the polymer chain. Were this to occur with the Polydyes the fluorescence efficiency of PD20 would be lower than that of PD80 in view of the 4-fold higher concentration of fluorophore monomers in the former and hence the greater potential for block copolymer formation.

Relative Fluorescence as a Function of Optical Density

Relative fluorescence intensity was measured for different polydyes and the bis-amino Cy3 monomer, as a function of optical density. The data are shown in FIG. 6.

The identical behaviour of PD20 and PD80 demonstrates that there is no variation in fluorescence efficiency of these dyes over this range of low bis-amino Cy3 incorporation and supports the view that the fluorescence behaviour of these materials is not influenced by dye association as would occur with non-random polymers.

Spectrophotometric Titrations

Simple and reproducible techniques for characterising the molecular conformation of hydrophobically modified polyelectrolytes of the type proposed are known. It has been established previously from potentiometric titration that poly(L-lysine iso-phthalamide) undergoes a hypercoiling transition between pH 4 and 5 (see, e.g., Eccleston et al., 1999). Such a transition is characterised by the collapse of the polymer conformation into a tight coil that is stabilised by hydrophobic association. Thereby, the environment of an associated or co-polymerised fluorophore is significantly changed and they are brought into closer proximity, effectively increasing their local concentration.

Aqueous solutions of polymers were titrated by with 1.0 N HCl delivered to an Aminco-SDP-125 spectrofluorimeter, equipped with a flow through cell, from a titration vessel, via a peristaltic pump.

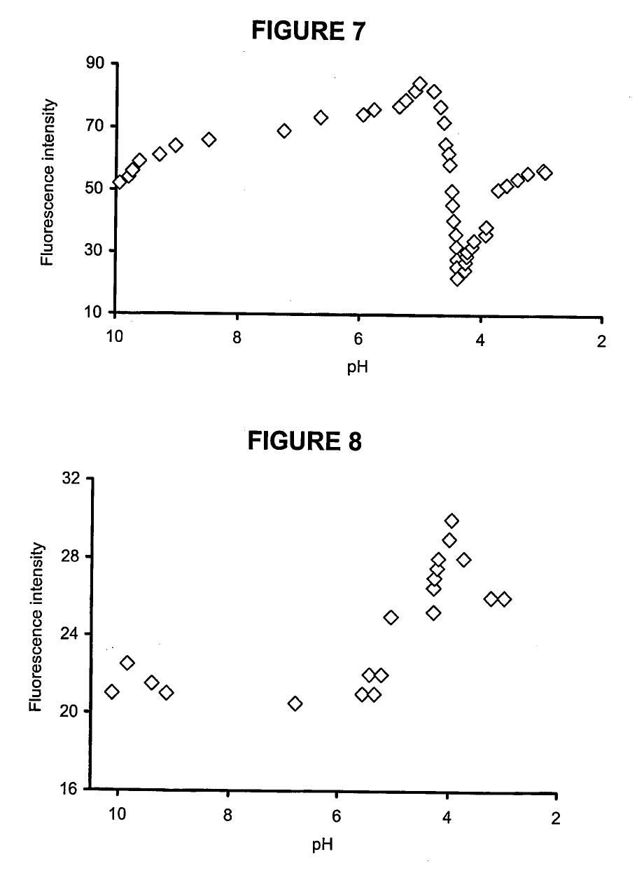

Spectrophotometric titrations were carried out for a mixture of poly(L-lysine iso-phthalamide) at a concentration of 0.5 g/l and free bis-amino Cy3 at a fluorophore concentration of 0.26 mM (0.1 g/l), which yields the maximum emission intensity for this polymer/fluorophore combination. The results are shown in FIG. 7.

A small initial rise in relative fluorescence intensity of the non-polymerised fluorophore, bis-amino Cy3, was observed as the pH fell below the pKₐ of the bis-amino groups (9.5), enabling increasing association of cationic fluorophore to the anionic polymer. There was a gradual increase in fluorescence output over the pH range 9.5 to 5.0 followed by a rapid decrease in the relative fluorescence intensity in the pH range 5.25-4.3 which corresponds to the pH range over which poly(L-lysine iso-phthalamide) changes conformation. The relative fluorescence intensity of the free fluorophore then increased again following precipitation of the polymer at around pH 4.25.

Spectrophotometric titrations were also carried out for a mixture of poly(L-lysine iso phthalamide) at a concentration of 0.5 g/l and free anionic bis-sulphonic acid Cy3 derivative at a fluorophore concentration of 0.16 mM (2.5 μg/mL). The results are shown in FIG. 8.

In contrast to bis-amino Cy3, spectrophotometric titration of the anionic bis-sulphonic acid Cy3 derivative in the presence of poly(L-lysine iso-phthalamide) showed no similar increase in relative fluorescence intensity with pH until pH 5, when loss of charge on the polymer enables hydrophobic dye-polymer association. In this case, there was also no marked reduction in relative fluorescence intensity when the polymer conformation collapses.

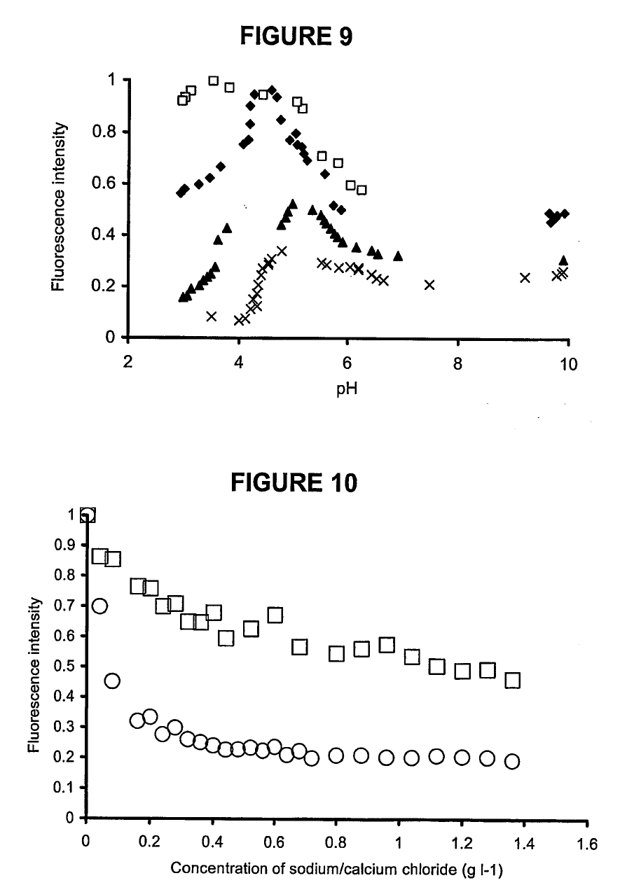

Spectrophotometric titrations were also carried out for aqueous solutions of the various polydyes (PD20, PD40, PD60, PD80), using 1.0 N HCl delivered to an Aminco-SDP-125 spectrofluorimeter (Thermo Spectronic, Rochester, N.Y., United States), equipped with a flow through cell, from a titration vessel via a peristaltic pump. Relative intensities are normalised to PD80 with a fluorophore concentration of 0.58×10⁻⁵ M. The results are shown in FIG. 9.

When the bis-amino Cy3 fluorophore was covalently bound within the backbone of the poly(L-lysine co-bis-amino-Cy3 iso-phthalamide), broad similarities were observed in the variation of relative fluorescent intensity with solution pH. No increase in relative fluorescence intensity was observed around the pKₐ of the free fluorophore since the amine groups were converted to amide linkages. Similarly, there is little increase in the relative fluorescence intensity observed on precipitation of the polymer since the fluorophores cannot be released into solution. These differences result from the conjugation of the fluorophore within the polymer.

The spectrophotometric titrations of the polydyes reveal a trend. As the amount of fluorophore is decreased the magnitude of the reduction in relative fluorescence intensity following polymer collapse decreases and is virtually absent in the case of PD80. This observation is consistent with the reduction in relative fluorescence intensity arising from the association of fluorophores on or within the polymer as it collapses. At lower degrees of substitution, the occurrence of multiply labelled polymers is diminished and the chance of intra-molecular fluorophore aggregation is thereby reduced. The spectrophotometric titration of PD80 is similar to that of the unlabelled poly(lysine iso-phthalamide) in the presence of the bis-sulphonic acid Cy3 derivative, displaying only a small decrease in relative fluorescence intensity prior to precipitation.

These observations are consistent with those of Rutkaite et al., 2001, who noted similar pH dependent variations in the fluorescence behaviour of amphiphilic carbazolyl-containing polymethacrylate copolymers containing a range of fluorophores. Polymers with low fluorophore content (1% and less) showed increased fluorescence at low pH due to reduced exposure of the fluorophore to external quenchers in the coiled state. At higher degrees of fluorophore loading (9-54% w/w) this effect was masked by enhanced self-quenching due to hydrophobic association of the fluorophores within the collapsed polymer. If sufficiently high amounts of fluorophore were included (65%), then no effect due to pH was seen, as the fluorophores were self-quenched at all pHs. Differences in the photo-physical properties of the carbazolyl-containing polymethacrylate polymers and the cyanine containing poly(lysine iso-phthalamide) polymers investigated here, indicate that the latter undergo pH mediated self-quenching at much lower fluorophore loadings. For the cyanine containing materials quenching was observed at fluorophore to repeat unit ratios of about 2.1%.

Salt Concentration

Relative fluorescence was measured for an aqueous solution of bis-amino Cy3 (0.26 mM) and poly(lysine iso-phthalamide) (0.5 g/l) using a Cytofluor plate reader (λₑₓ 535 nm and λₑₘ 570 nm) as a function of sodium chloride (□) (0-1.4 g/L) and calcium chloride (◯) concentration (0-1.4 g/L). The results are shown in FIG. 10.

It was observed that the relative fluorescence intensity of the mixed polymer/fluorophore samples were strongly dependent on the concentration of added salts. In the presence of sodium chloride a near linear relationship between relative fluorescence intensity and salt concentration was observed, whereas in the presence of divalent cations such as calcium chloride a rapid drop in the relative fluorescence intensity was seen at low salt concentrations. The decrease in relative fluorescence intensity at higher ionic strength presumably arises from the promotion of polymer collapse by increased charge shielding at higher salt concentrations. See, e.g., Reddington, 1998. In the case of the divalent calcium cation the effect is enhanced by the ability of multivalent ions to coordinate several carboxyl groups.

Biological Methods

Cell Lines

The following cell lines were employed in the biological studies:

C26 (Murine colon adenocarcinoma), obtained from the Cancer Research Centre (CRC) at the University of Birmingham, Birmingham, UK.

A2780 (Human ovarian carcinoma), obtained from the Cancer Research Centre (CRC) at the University of Birmingham, Birmingham, UK.

COS1 (African green monkey kidney), obtained from the Cancer Research Centre (CRC) at the University of Birmingham, Birmingham, UK.

Epstein-Barr virus-transformed B cell line (EBV-transformed lymphoblastoid cells from normal adult donors), obtained from the Cancer Research Centre (CRC) at the University of Birmingham, Birmingham, UK.

CHO (Chinese hamster ovarian), obtained from Amersham Biosciences, Cardiff, UK.

HepG2 (Human hepatocyte carcinoma), obtained from Amersham Biosciences, Cardiff, UK.

Culture Conditions

All tissue culture manipulations were carried out in sterile laminar flow hoods, using sterile disposable plasticware. Anything introduced into the culture vessels was either autoclaved or filter (0.2 μm) sterilised.

C26, COS1 and HepG2 cell lines were grown in Dulbecco's Minimum Eagle medium, (DMEM) (Invitrogen Life Technologies Ltd, Paisley, Scotland, UK) containing 10% foetal bovine serum (FBS) (Sigma, Poole, Dorset, UK).

CHO cells were grown in Nutrient Mixture F-12 Ham medium (Sigma, Poole, Dorset, UK).

A2780 cells were grown in RPMI-1640 medium (Sigma, Poole, Dorset, UK) supplemented with 10% FBS (Sigma, Poole, Dorset, UK).

Serum was supplied heat-inactivated to avoid complement-mediated lysis of cultured cells. All media was supplemented with 100 U/mL penicillin and 100 μg/mL streptomycin (Sigma, Poole, Dorset, UK) to discourage the growth of micro-organisms and 200 mM L-Glutamine (Sigma, Poole, Dorset, UK). The cell lines were maintained in a humidified incubator at 37° C. with 95% air and 5% CO₂.

The suspension cell line, EBV-transformed B cell line, was cultured in RPMI-1640 medium containing 10% FBS (Sigma, Poole, Dorset, UK), with 100 U/mL penicillin and 100 μg/mL streptomycin (Sigma, Poole, Dorset, UK). The cells were subcultured twice weekly by re-suspending the cells by agitation and then replacing half the cell volume with fresh medium.

Adherent cells were grown to confluence in tissue culture grade flasks (75 cm²), and were subcultured by discarding the old medium, leaving the cells adhered to the bottom of the flask. The cells were then washed with 5 mL of Dulbecco's Phosphate Buffered Saline (Sigma, Poole, Dorset, UK), and then incubated at 37° C. with 3 mL of trypsin-EDTA (Sigma, Poole, Dorset, UK) until detached from the bottom of the cell culture flask. 3 mL complete medium (containing 10% FBS, 2 mM L-Glutamine and 100 U/mL penicillin and 100 μg/mL streptomycin) was added to neutralise the trypsin, and typically one tenth of the cells were retained for further sub-culture.

Determination of Cell Concentration

The number of suspension or trypsinised adherent cells was estimated using an improved Neubuaer-haemocytometer (Fison Scientific Equipments, Loughborough, UK). A sample of the cell suspension was introduced onto the haemocytometer covered with a glass coverslip giving a gap of 0.1 mm. The cells within the central 25 squares (1 mm²) were counted using a trans-illuminated Olympus CK2 microscope (Olympus Optical Co., Tokyo, Japan) fitted with a 10× objective lens. The cell count multiplied by 10⁴ gives the number of cells per mL (cm³).

Viable Cell Counts (Trypan Blue Staining)

The trypan blue staining technique was used to distinguish between viable and non-viable cells on the haemocytometer. 1 mL of cell suspension was mixed with 1 mL 0.4% trypan blue stain (Sigma, Poole, Dorset, UK), and applied to a Neubuaer-haemocytometer. The number of cells counted (ignoring blue/dead cells) is multiplied by 2×10⁴ to calculate cells/mL to take into account the dilution factor upon addition stain.

Cell Storage

All cell lines were frozen and stored in liquid nitrogen. 2-3×10⁶ cells were centrifuged at 1000 rpm (180×g) in a Hettich universal 30 RF bench-top centrifuge (Scientific Laboratory Supplies Ltd., Wilford, Nottinghamshire, UK). The supernatant was discarded, and the cell pellet re-suspended in 1 mL of freezing mix (70% medium, 20% FBS, 10% DMSO). The cells were transferred to a cryogenic vial (Nalgene), put into a Nalgene's Mr. Frosty (Fison Scientific Equipments, Loughborough, UK), filled with isopropanol (pre-cooled to 4° C.) and placed in a −70° C. freezer overnight (24 hours). The cryogenic vials were then transferred to liquid nitrogen for long-term storage.

The suspension cell line, EBV-transformed B cell line was pelleted by centrifugation at 1000 rpm (180×g) in an MSE bench-top centrifuge. The supernatant was discarded and the pellet was re-suspended in freezing medium (50% FBS: 40% medium: 10% DMSO).

Frozen cells were thawed in an incubator at 37° C. for four minutes. The cells were then mixed with 20 mL of pre-warmed medium (37° C.) to dilute the DMSO, and centrifuged for 3 minutes at 3000 rpm. The supernatant was discarded, the pellet re-suspended in fresh medium (10 mL), and the cells cultured as usual. The cell culture flask was checked 24 hours later for cell growth and the medium changed if it contained substantial amounts of cell debris. Cells were sub-cultured twice prior to use.

Cytotoxicity: MTT Assay

The cytotoxic effects of test polymers towards a C26 cell line were tested in vitro at physiological pH (7.4) by measuring mitochondrial dehydrogenase activity using 3-{4,5-dimethylthiazol-2-yl}-2,5-diphenyltetrazolium bromide (MTT, Sigma, Poole, Dorset, UK). The assay has gained widespread favour for the quantitation of cell viability. The metabolism of the tetrazolium salt to a dark purple formazan product gives a rapid assessment of cell survival and proliferation. However, the MTT assay cannot always be employed as the method of choice as not all cells have the ability to metabolise MTT and others appear to lose the ability at high densities (Coley et al., 1997).

Aliquots (200 μL) of colon-C26 cells (4×10³ cells/well) were dispensed into 96-well microtitre plates (Corning, Bibby Sterilin Ltd., Staffordshire, UK) and incubated overnight in a humidified incubator at 37° C. (95% air and 5% CO₂). The supernatant was then removed and replaced with 200 μL serial dilutions of test polymer (2-500 μg/mL polymer/medium in quadruplicate). Control wells (cells and medium only (200 μL) were also prepared in quadruplicate.

The plates were incubated for 2 hours then the medium containing the polymer was removed and the plates washed once with phosphate buffered saline (PBS). 200 μL of fresh medium was added to each well and the plates incubated for a further 48 hours. A standard MTT assay was then performed (Sigma, Poole, Dorset, UK). 20 μL of a 5 mg/mL MTT solution (dissolved in PBS and filter sterilised) was added to each well. The plates were then incubated for 5 hours and the supernatant removed using a fine needle, taking care not destroy the MTT crystals. 200 μL of DMSO was added to each well and complete solubilisation of formazan crystals was achieved by repeated pipetting of the solution. The plates were then read using an Anthos 2001 plate reader (Labtech International Ltd, Ringmer, East Sussex, UK) with a 550 nm filter. The cytotoxicity of the test polymer was expressed as the relative cell viability as a percentage of the control according to the following equation:
Relative viability (%)=[(AP−AB)/(AC−AB)]×100%.
wherein AP denotes absorbance measured in the for polymer treated samples, AB denotes background absorbance, and AC denotes absorbance of untreated control samples.

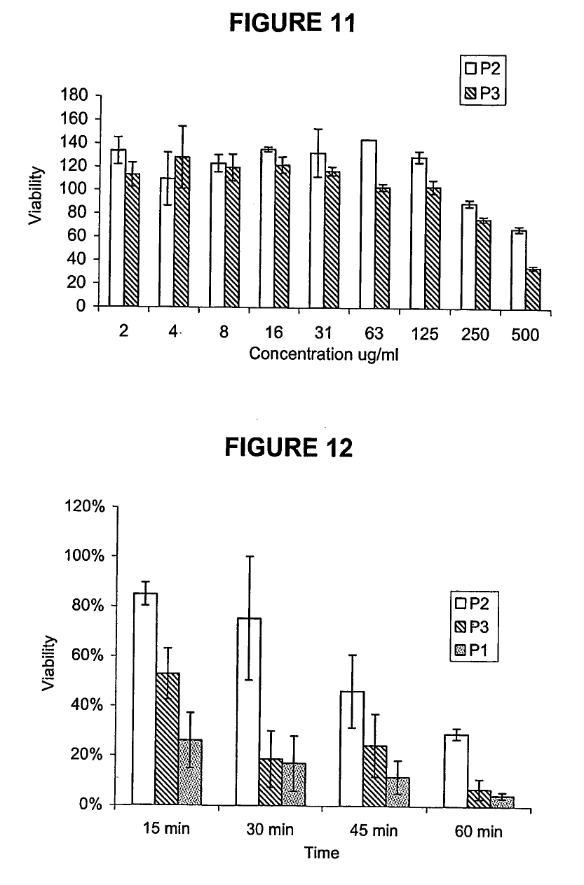

MTT cytotoxicity data for naked polymer (poly(L-lysine iso-phthalamide)) (P3) and poly L-lysine ethyl ester co-L-lysine iso-phthalamide) (P2) are shown in FIG. 11.

Both polymers were well tolerated at physiological pH (pH 7.4) up to 125 μg/mL. There was some indication of increased cell growth in the presence of the polymers. This could be due to cells utilising low molecular weight oligomers, present in the unfractionated samples, as a nutrient source.

At concentrations of 250 and 500 μg/mL, the cell viability dropped by 24% and 65%, respectively, in the presence of poly(L-lysine iso-phthalamide). At concentrations of 250 and 500 μg/mL, the cell viability dropped by 10.2% and 32%, respectively, in the presence of poly(L-lysine ethyl ester-co-L-lysine iso-phthalamide).

Cell Lysing: Lactate Dehydrogenase (LDH) Release Assay

LDH activity was determined using a CytoTox 96® assay kit (Promega Corporation, Hampshire, UK), which quantitatively measures lactate dehydrogenase (LDH), a stable cytosolic enzyme that is released upon cell lysis. LDH released into the culture supernatant is measured with a 30 minute coupled enzymatic assay that converts a tetrazolium salt (INT) into a red formazan product. The optical absorption (measured at 492 nm, and corrected for background absorption) is directly proportional to the number of lysed-cells.

The lactate dehydrogenase (LDH) activity of COS1 and A2780 cells was determined as follows. A2780 and COS1 cell lines were plated onto 96-well plates with varying densities (0-20,000 cells/well) and incubated for 24 hours. The tissue culture medium was removed and replaced with serum-free medium (200 μL/well). The cells were then frozen at −80° C. and slowly thawed at 37° C. to effect cell rupture. 50 μL aliquots from each well were dispensed in mirror fashion into fresh 96-well plates. To each well, 50 μL of the substrate reaction mixture from the CytoTox 96® assay kit was added and the colour reaction allowed to develop for approximately 30 minutes in the dark. 50 μL of stop solution from the CytoTox 96® assay kit was then added to all wells and the absorbance was read within 1 hour at 492 nm using an Anthos 2001 plate reader (Labtech International Ltd, Ringmer, East Sussex, UK).

The absorbance was plotted versus cell density to demonstrate a linear response over the range studied.

Lysing Extracellular Membranes: COS1 Cells

The ability of test polymers to lyse the extracellular membranes of COS1 cells, (a) at pH 7.4, and (b) following acidification of cell culture supernatants to pH 5.5, was tested in vitro using the LDH release assay.

Approximately 5×10³ COS1 cells (100 μL) were incubated with 1-500 μg/mL of test polymer. After one hour, the cells were pelleted by centrifugation in a Heffich universal 30 RF bench-top centrifuge equipped with a 96-well plate accessory (Scientific Laboratory Supplies Ltd., Wilford, Noffinghamshire, UK) at 1000 rpm for 4 minutes, and the amount of LDH released into the medium was measured using the LDH release assay described above, and expressed as a percentage of the control according to the following equation:
Relative viability (%)=[(AP−AB)/(AC−AB)]×100%.
wherein AP denotes absorbance measured in the for polymer treated samples, AB denotes background absorbance, and AC denotes absorbance of untreated control samples. Cytotoxicity was calculated according to the following equation:
Cytotoxicity (%)=100%−Relative viability (%)
No cytotoxicity was observed with any polymer at concentrations up to 100 μg/mL.

Measured cytotoxicities at 500 μg/mL are summarised in the following table; however, the assay wells appeared turbid due to polymer precipitation caused by the acetic acid in the “stop” solution.

TABLE 3
Cytotoxicity at 500 μg/mL

| Polymer | Cytotoxicity |
|---|---|
| poly (L-lysine dodecanamide | 14.95% |
| poly (L-lysine ethyl ester- | 6.7% |
| co-L-lysine iso-phthalamide | |
| poly (L-lysine iso-phthalamide | 13.7% |

A modified version of the LDH release assay was developed to circumvent turbidity problems. In the adapted protocol, the LDH release assay was used to estimate the number of cells left intact at the end of the assay (cell viability), as opposed to the number of cells lysed. By performing the assay in this way turbidity problems were removed from the assay.

In the modified assay, approximately 1×10⁴ cells per well were seeded in a 96-multiwell plate (Corning, Bibby Sterilin Ltd., Staffordshire, UK) and incubated overnight (24 hours) in Dulbecco's Minimum Eagle medium, (DMEM) (Invitrogen Life Technologies Ltd, Paisley, Scotland, UK) containing 10% foetal bovine serum (FBS) (Sigma, Poole, Dorset, UK) at 37° C. in a humidity controlled incubator in an air atmosphere supplemented with 5% CO_{2.} The medium was then removed from the wells and the cells were washed three times with 100 μL Dulbecco's Phosphate Buffered Saline (Sigma, Poole, Dorset, UK), to remove excess serum. 100 μL of fresh Dulbecco's Minimum Eagle medium, (DMEM) (Invitrogen Life Technologies Ltd, Paisley, Scotland, UK) containing 1 mg/mL solution of test polymer was added to the wells in quadruplicate. Control wells containing COS1 cells in 100 μL serum-free medium were also prepared in quadruplicate.

The pH of each well was adjusted immediately to pH 5.5, by titration with 0.1 M HCl (27.5 μL) and the cells incubated for the appropriate time-course (15, 30, 45, or 60 minutes). Following the appropriate incubation periods the medium was removed from each well and replaced with 100 μL serum-free medium (Dulbecco's Minimum Eagle medium, (DMEM) (Invitrogen Life Technologies Ltd, Paisley, Scotland, UK). The plate was then frozen in a −80° C. freezer for 45 minutes to effect cell lysis, and then thawed for 15 minutes at 37° C.

The plate was then centrifuged for 4 minutes (1000 rpm) and 50 μl of supernatant was transferred to each well of a newly prepared 96-multiwell flat-bottomed plate contained fresh serum free medium. 50 μl of the substrate reaction mixture from the CytoTox 96® assay kit was added to each well and the plate wrapped in tin foil for 30 minutes to allow the red coloured product formed. At this point, 50 μl of stop solution from the CytoTox 96® assay kit was added to each well and the plates incubated at 37° C. for 5 minutes to eliminate bubbles. The absorbance of the wells were measured at 490 nm using an Anthos 2001 plate reader (Labtech International Ltd, Ringmer, East Sussex, UK) and the results presented as cell viabilities as a percentage of the control according to the following equation:
Relative viability (%)=[(AP−AB)/(AC−AB)]×100%.
wherein AP denotes absorbance measured in the for polymer treated samples, AB denotes background absorbance, and AC denotes absorbance of untreated control samples.

COS1 relative cell viability data for poly(L-lysine dodecanamide) (P1), poly(L-lysine ethyl ester co-L-lysine iso-phthalamide) (P2), and poly(L-lysine iso-phthalamide) (P3) are shown in FIG. 12.

Upon acidification of the supernatant, the overall COS1 cell viability dropped markedly even in the absence of polymer; however, clear differences were observed between control (no polymer) and in the presence of each of the test polymers. For each test polymer, the viability of cells relative to control fell with time with a minimum viability of only 4% for treatment with poly(L-lysine dodecanamide) after 60 minutes.

Cell viability studies using A2780 cell were performed using analogous methods, with the exception that the A2780 cells were incubated with the polymers for 30 minutes and then re-suspended in 100 μl fresh serum-free medium prior to pH adjustment to 5.5 with 1.0M HCl. The A2780 cells were then incubated for the appropriate time course (15, 30, 45, or 60 minutes) and the remaining live cells quantified using the modified LDH release assay.

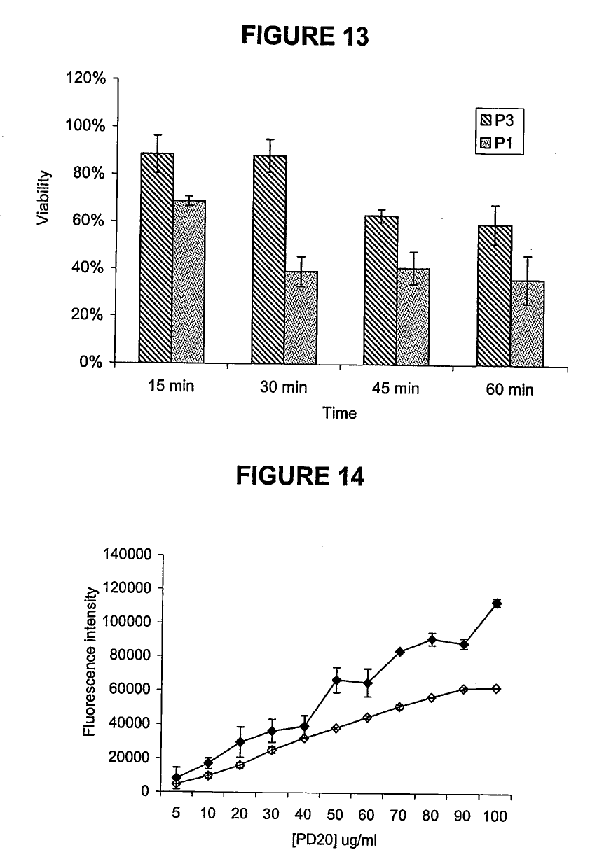

A2780 relative cell viability data for poly(L-lysine dodecanamide) (P1) and poly(L-lysine iso-phthalamide) (P3) are shown in FIG. 13.

The A2780 cells were similarly insensitive to polymer treatment at pH 7.4 but showed a pH dependent reduction in viability in the presence of the test polymers. The minimum viability was higher than for COS1 cells, at 36% after 60 minutes exposure to poly(L-lysine dodecanamide).

Fluorescence Intensity in the Presence of Serum Proteins

The effect of serum proteins on the fluorescence of test polymers was investigated.

Fluorescence intensities were measured for solutions of test polymer in Dulbecco's Phosphate Buffered Saline Sigma, Poole, Dorset, UK) with 10% foetal bovine serum (FBS) (Sigma, Poole, Dorset, UK) and serum free Dulbecco's Phosphate Buffered Saline (Sigma, Poole, Dorset, UK) using a Wallac-Victor 1420 fluorescent plate reader (Perkin Elmer Instruments, Beaconsfield, Buckinghamshire, UK) with λₑₓ 535 nm and λₑₘ 590 nm.

Fluorescence intensity was measured for each of PD20, PD40, PD60 and PD80, at a concentration of 60 μg/mL, as a function of pH (over the range pH 4.0-7.4).

Fluorescence intensity was also measured for PD20, at concentrations ranging from 5-100 μg/mL, at pH 7.4.

The fluorescence intensity of an aqueous solution of PD20, in the presence of serum, is higher over a range of concentrations than in the absence of serum. See FIG. 14.

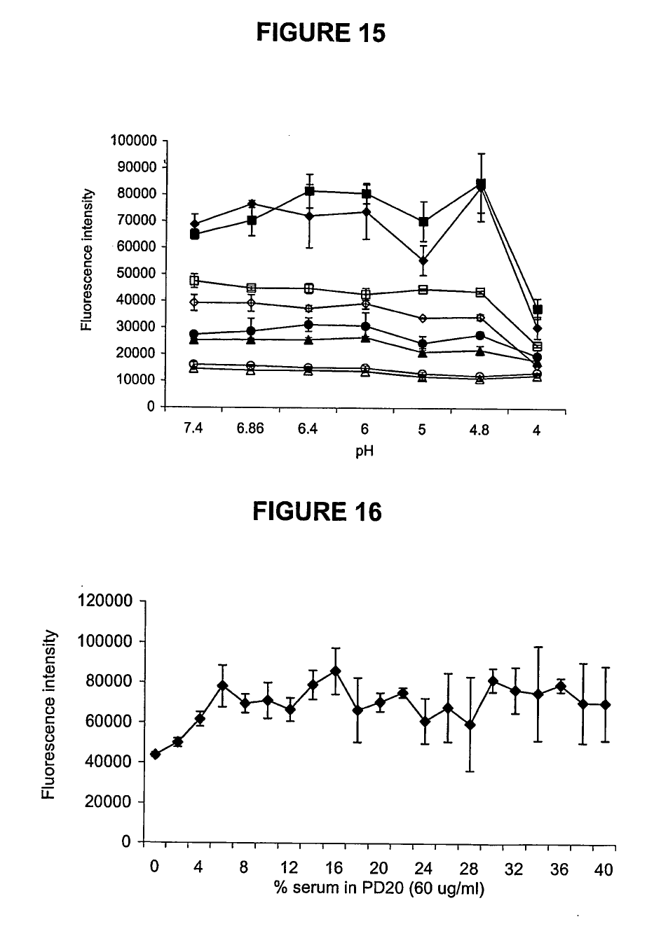

This is due to interaction of the polydye with serum proteins with a consequent “rigidising” effect on the fluorophore. This leads to a reduction in non-radiative loss processes that, in the absence of serum, result in a reduced quantum yield. The enhancement occurs over a broad range of pH until the polymer begins to precipitate from solution, at which point interaction with the serum proteins is reduced and the fluorescence approaches that of the polymer in the absence of serum. See FIG. 15.

It can be seen from FIG. 16 that, at levels of serum proteins above approximately 5%, there is no further enhancement in the fluorescence at this concentration of polydye.

Association with Scintillation Proximity Assay (SPA) Beads

The interaction of test polymers with hydrophobic and hydrophilic scintillation proximity assay (SPA) beads was examined.

Hydrophobic and hydrophilic SPA beads were obtained as a suspension (104 mg/g) in PBS/sodium azide (Hydrophobic Polymer Laboratories, Church Stretton, Shropshire, UK).

10 μL of SPA bead suspension and 500 μL of test polymer at a concentration of 1 mg/mL in PBS titrated to various pHs with 1.0 M HCl were added to independent Willco dishes (Intracel, Royston, UK) in quadruplicate and allowed to settle for 10 minutes. Images of the beads at the various pH values were obtained using a Laser Scanning Confocal Zeiss LSM410 Microscope, λₑₓ 535 nm, λₑₘ 570 nm (Amersham Biosciences, Cardiff, UK) and their fluorescence intensity quantified using Metamorph™ software.

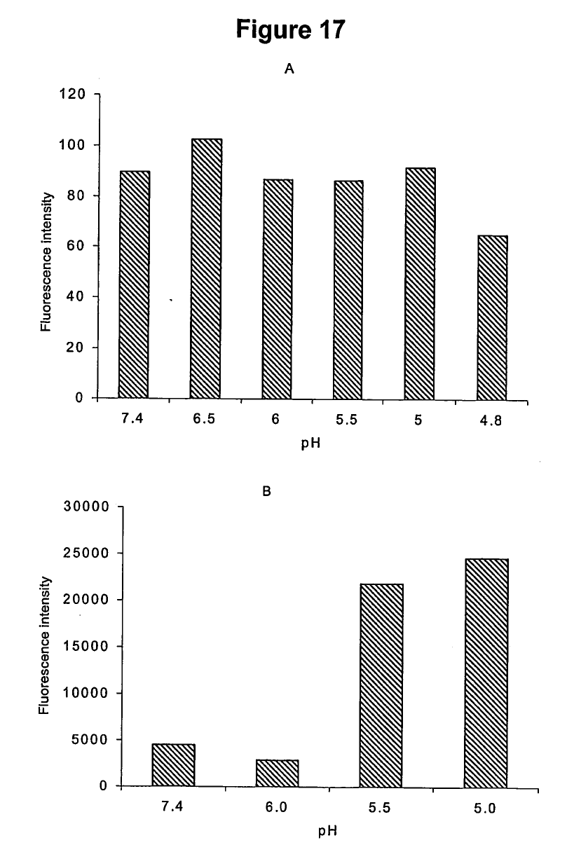

There is very little interaction between a representative polydye hydrophilic state and hydrophilic or hydrophobic SPA beads when the polymer is charged. However, upon loss of charge, the polymer becomes increasingly hydrophobic and interacts strongly with the hydrophobic SPA beads. See FIG. 17.

The pH-dependant behaviour of the polydye with hydrophilic and hydrophobic SPA beads was used as a model for the interaction of the polymeric fluorophore with cellular membranes.

Interaction of PD20 with CHO cells at pH 7.4 and pH 5.5

A 2 mL suspension of CHO cells (10⁵ cells/mL) was seeded into WilCo dishes (Intracel, Royston, UK) and incubated overnight (24 hours) at 37° C. in a humidity controlled incubator in an air atmosphere supplemented with 5% CO₂.

The CHO cells were then washed three times with 100 μl of D-PBS (Sigma, Poole, Dorset, UK) to remove excess serum. 2 mL of PD20 (1 mg/mL) in D-PBS (Sigma, Poole, Dorset, UK) at pH 7.4 or titrated to pH 5.5 with 0.1 M HCl was then added to the Willco dishes.

The dishes were incubated for 30 minutes, the PD20 removed (by aspiration) and the cells washed three times with 2 mL D-PBS (Sigma, Poole, Dorset, UK) adjusted to pH 7.4 or pH 5.5 with 1.0 M HCl.

The CHO cells were then re-suspended in 2 mL of D-PBS at pH 7.4 or titrated to pH 5.5 with 0.1 M HCl and fluorescent photomicrogram images obtained on a Laser Scanning Confocal Zeiss LSM410 microscope (λₑₓ 535 nm, λₑₘ 570 nm) with a ×20 magnification lens (Amersham Biosciences, Cardiff, UK).

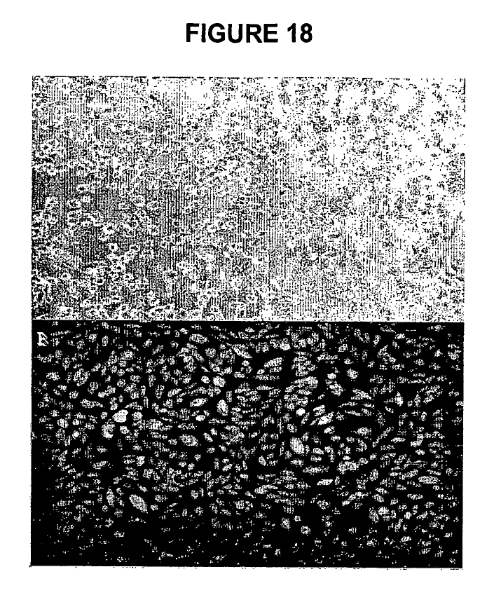

Phase contrast images of the CHO cells were obtained on the same microscope in non-fluorescence mode. The images are shown in FIG. 18.

The fluorescence microscopy images indicated that at pH 7.4, when the polymer is extensively charged (see FIG. 16(A)), PD20 was only weakly adsorbed onto the membrane of CHO cells, which is also negatively charged, following a 30 minute incubation period at 37° C.

By contrast, incubation under the same conditions at pH 5.5, when the polymer begins to display increased hydrophobic characteristics (see FIG. 16(B)), led to extensive adsorption of the polymer onto the extracellular membrane of the CHO cells.

Interaction of PD20 with CHO Cells at pH 7.4, 6.0 or 5.0

CHO cells (80,000/mL) were seeded into WilCo dishes and incubated overnight (24 hours) at 37° C. in a humidity controlled incubator in an air atmosphere supplemented with 5% CO₂.

The CHO cells were then washed three times with 2 mL D-PBS (Sigma, Poole, Dorset, UK) to remove excess serum and incubated with 2 mL PD20 (1 mg/mL) in serum-free medium (F12 Nutrient Ham Mixture, Sigma, Poole, Dorset, UK) titrated to pH 7.4, 6.0, or 5.0 with 0.1 M HCl.

The CHO cells were then incubated for 10 minutes and washed three times with 2 mL D PBS (pH 7.4, 6.0, or 5.0). 2 mL of fresh D-PBS at the required pH (pH 7.4, 6.0, or 5.0) was added to each dish and the CHO cells were then imaged on a Zeiss LSM410 confocal microscope (Amersham Biosciences, Cardiff, UK) for 1 hour at 10 minute intervals (to reduce photobleaching and cell exposure to the laser). The cells were maintained at 37° C. on an integral heater stage.

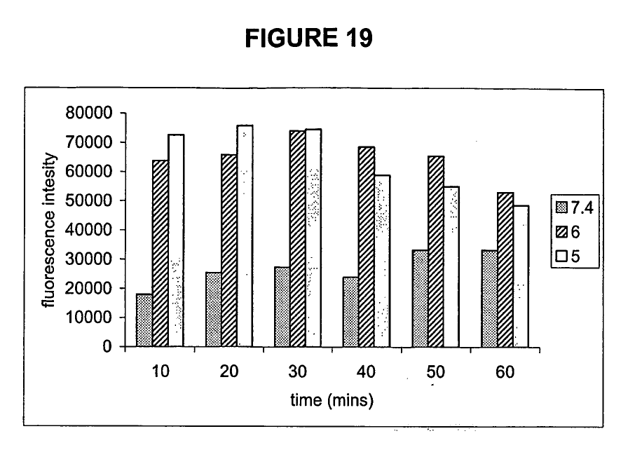

The confocal microscope images were quantified using Metamorph™ software to give the fluorescence intensity. Fluorescence intensity data are shown in FIG. 19.

There was a gradual increase in the relative fluorescence intensity of the CHO cells with time when exposed to PD20 at pH 7.4, whereas at pH 6.0 and pH 5.0 the fluorescence intensity peaked at 30 minutes and 20 minutes respectively, and then began to decrease. The relative fluorescence intensity of the CHO cells at pH 6.0 and pH 5.0 was initially more than three times larger than those exposed to P20 at pH 7.4.

Co-Localisation of PD20 with FITC-Alexa

HepG2 cells (80,000/mL) were seeded into WilCo dishes, and incubated overnight (24 hours) at 37° C. in a humidity controlled incubator in an air atmosphere supplemented with 5% CO₂.

The HepG2 cells were then incubated for a period of 15 minutes with a mixture of PD20 at a concentration of 1 mg/mL and the endosomal labelling fluorophore FITC-Alexa Fluor 488 (Cambridge Biosciences, Cambridge, UK) at a concentration of 50 μg/mL in serum free medium (Dulbecco's Modified Eagle Medium, DMEM) appropriate for HepG2 cells (total volume 2 mL) at pH 7.4.

The supernatant was then removed and the cells washed three times with 2 mL DMEM. 2 mL of fresh D-PBS was added to each dish and the CHO cells were then imaged on a Zeiss LSM410 confocal microscope (λₑₓ 535 nm, λₑₘ 570 nm for PD20, λₑₓ 495 nm, λₑₘ 518 nm for FITC Alexa Fluor 488) (Amersham Biosciences, Cardiff, UK).

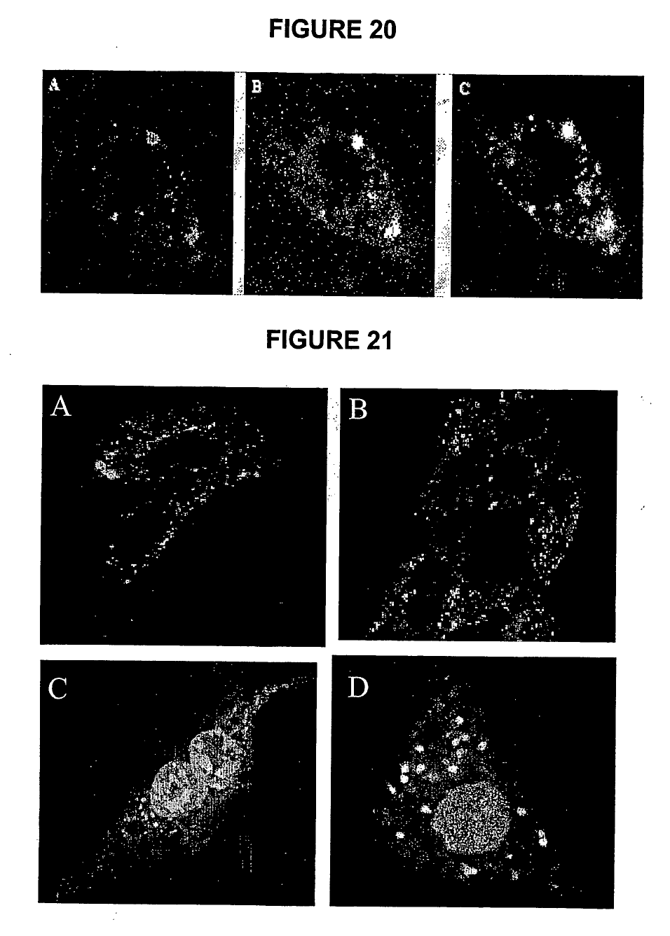

The confocal microscope images are shown in FIG. 20. The first image (A) showed a punctuate staining pattern within the cell wall. The second image (B) showed a similar pattern. When the two images are combined, to give image (C), the staining patterns completely overlap, indicating uptake of the PD20 into the endosomal compartment.

Uptake and Internalisation of PD30 into CHO cells

CHO or HepG2 cells (80,000 cells/mL) were seeded into Willco dishes and incubated overnight (24 hours) at 37° C. in a humidity controlled incubator in an air atmosphere supplemented with 5% CO₂.

The cells were then washed three times with 2 mL D-PBS (Sigma, Poole, Dorset, UK) to remove excess serum. 2 mL of PD30 at a concentration of 1 mg/mL in D-PBS (pH 7.4) was added, and the cells incubated for 10 minutes at 37° C. in a humidity controlled incubator in an air atmosphere supplemented with 5% CO₂.

Controls were prepared similarly, by the addition of 2 mL of bis-amino Cy3 at a concentration of 0.01 mg/mL in D-PBS (pH 7.4), or 2 mL of bis-sulphonic acid Cy3 at a concentration of 0.01 mg/mL in D-PBS (pH 7.4), followed by incubation for 10 minutes at 37° C. in a humidity controlled incubator in an air atmosphere supplemented with 5% CO₂.

Laser Scanning Confocal Microscopy (LSCM) images were then obtained at 10 minute intervals, using an Olympus FV300 LSCM (Department of Chemical Engineering, University of Cambridge, Cambridge, Cambridgeshire, UK). The cells remained on the microscope for the duration of the imaging experiments.

The LSCM images for the CHO cells are shown in FIG. 21.

The first image (A) shows the uptake of the free bis-sulphonic acid Cy3, which is shown to be membrane bound. The second Image (B) shows the uptake of the free bis-amino Cy3, which is shown to be localised in the endosome. The third image (C) and the fourth image (D) show the uptake of PD30, which is shown to be localised in the nucleus.

The images of the cells exposed to the non conjugated dyes both show staining throughout the cytoplasm and endosomes of the cell but not in the nucleus. When exposed to the PD30 however, the cellular staining is confined mainly to the endosomes, with distinct bright spots, and the nucleus is also heavily stained. One of the images shows a brightly stained nucleus splitting into two during the process of mitosis.

The images demonstrate that, in the case of the COS 1 cells, the free dyes (images A and B) are present within the cell and probably within endosomal compartments given the punctuate staining pattern.

In the case of the PD30, however, the staining is clearly associated with the nucleus (images C and D). In image C, the cell is undergoing mitosis. In addition, the cytoplasm is clearly stained indicating endosomal escape. In image D, there are multiple punctuate stained areas possibly indicating lysosomal compartments.

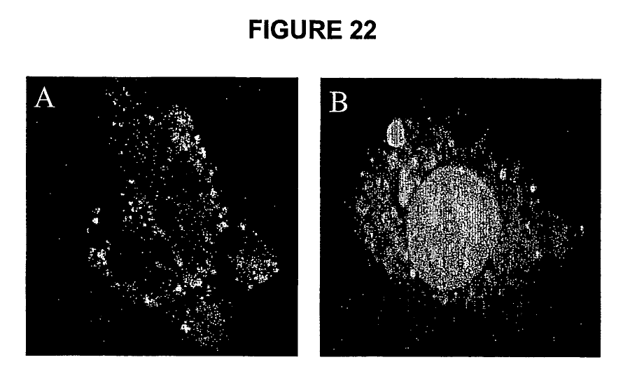

The LSCM images for the HepG2 cells are shown in FIG. 22.

The first image (A) shows the uptake of the free unconjugated fluorophore, bis-amino Cy3, which is shown to be localised in the endosome. The second image (B) shows the uptake of PD30, which is shown to be localised in the nucleus.

The image of the cell exposed to the unconjugated fluorophore (Image A) shows staining throughout the cytoplasm and within the endosomes but with no nuclear staining, whereas the image of the cell exposed to PD30 (Image B) shows heavy staining in the nucleus, even after just 30 minutes.

The images for HepG2 cells are similar to those for CHO cells. No nuclear staining was observed for the free bis-amino Cy3 (image A), and extensive nuclear staining was for PD30 (image B).

Polymer-Conjugated Doxorubicin: Uptake Studies

This study demonstrates the uptake into cells of doxorubicin (as the payload) covalently bonded (via an amide bond) to poly(L-lysine isophthalamide) (as carrier polymer) (see Illustrative Examples, above).

The membrane permeability of human breast cancer (MCF7) and mitoxantrone resistant (MCF7/MXR) cell lines cell lines towards (a) free doxorubicin and (b) poly(L-lysine iso-phthalamide)-conjugated doxorubicin (2% loading of doxorubicin on polymer via amide conjugation) was investigated in the absence and presence of 1% by volume of dimethyl sulphoxide (DMSO) (as a membrane permeation enhancer).

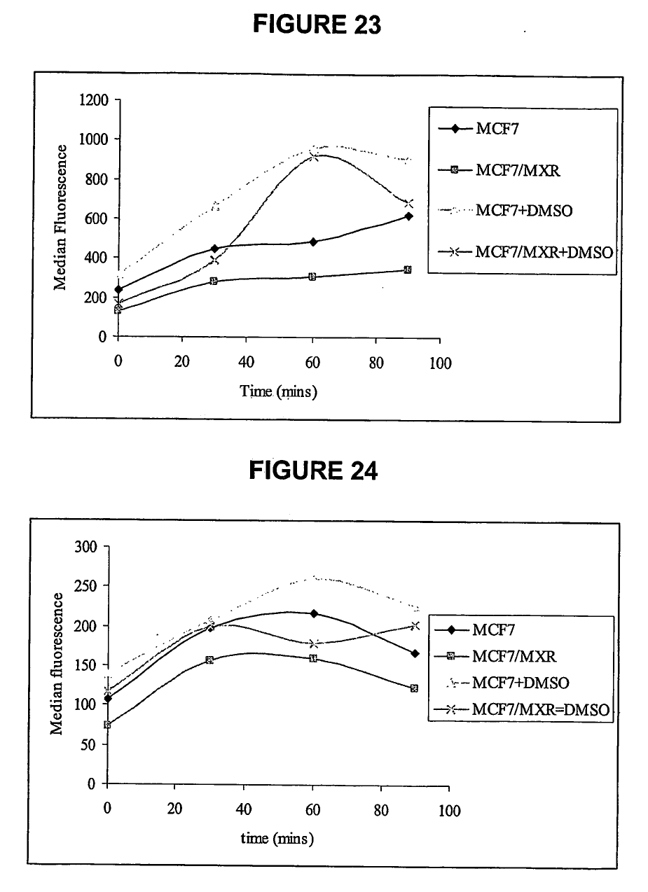

Test solutions were prepared at a concentration of 2 mM doxorubicin (or doxorubicin equivalent, in the case of polymer-conjugated doxorubicin) in foetal calf serum-free medium, with or without 1% by volume of DMSO. Control solutions, containing polymer (with no doxorubicin) and medium alone were also prepared. The polymer had a molecular weight of approximately 18 kDa. Samples (1 mL) of each test solution were placed in Eppindorf centrifuge tubes and placed in an incubator at 37° C. under a 5% CO₂ atmosphere. 2×10⁶ cells of each type were suspended in 300 μL of cell culture media (10% foetal calf serum, 2 mM L-Glutamine, 100 U/mL penicillin and 100 μg/mL streptomycin) and incubated at 37° C. under a 5% CO₂ atmosphere. The study was initiated by adding 10 μL of cells to each test sample. After selected exposure times, the cells were pelleted by centrifuge and placed on ice. The cells were then re-suspended in phosphate buffer and analysed using a flow cytometer gated to count 50 events (approximately 10% of the total cell count). A histogram of fluorescence intensity vs. number of events, and the median value of fluorescence intensity, was recorded for each sample. Background fluorescence for cells alone, or for cells with DMSO, was subtracted from the corresponding test samples. The results are illustrated in FIG. 23 (free doxorubicin) and FIG. 24 (polymer-conjugated doxorubicin).

The data demonstrates that both free doxorubicin and polymer-conjugated doxorubicin are taken up into cells. The presence of DMSO significantly enhances the rate of uptake, in keeping with the mode of entry, that is, diffusion across the cell membrane. Since the polymer-conjugated doxorubicin enters the cells by the process of endocytosis, the presence of DMSO in the tissue culture media has little effect on its uptake.

Polymer-Conjugated Doxorubicin: Cytotoxicity Studies 1

This study demonstrates that the biological activity of doxorubicin (as the payload) covalently bonded to poly(L-lysine iso-phthalamide) (as carrier polymer) is retained.

The cytotoxicity of (a) doxorubicin, (b) poly(L-lysine iso-phthalamide) conjugated doxorubicin and (c) poly(L-lysine iso-phthalamide) was assessed in human breast cancer (MCF7) and mitoxantrone resistant (MCF7/MXR) cell lines.

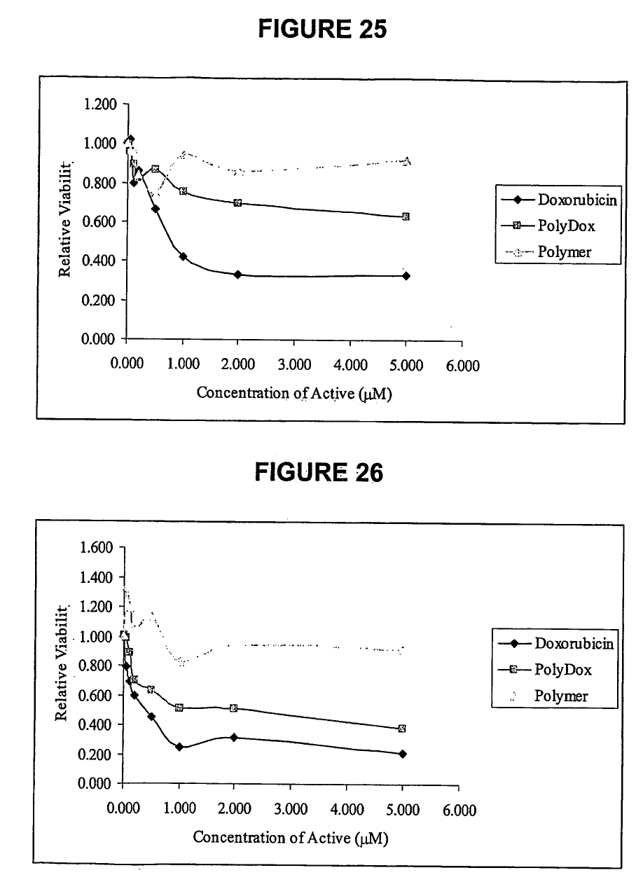

5×10³ cells of each type were seeded in wells of a 96 well plate in 200 μL tissue culture media (10% foetal calf serum, 2 mM L-glutamine, 100 U/mL penicillin, and 100 μg/mL streptomycin) together with doxorubicin, or polymer-conjugated doxorubicin, or polymer alone (in its carboxylate salt form), at particular concentrations, and stored in an incubator at 37° C. under a 5% CO₂ atmosphere for 5 days. The polymer had a molecular weight of approximately 18 kDa. Subsequently, a standard MTT (3-(4,5-dimethylthiazol-2-yl)-2,5-diphenyl-2H-tetrazolium bromide) assay was performed in order to assess cell viability. The results are illustrated in FIG. 25 (for MCF7 cells) and FIG. 26 (for MCF7/MXR cells).

The data demonstrates that both free doxorubicin and polymer-conjugated doxorubicin exhibit significant cytotoxic effects.

Polymer-Conjugated Doxorubicin: Cytotoxicity Studies 2

The cytotoxicity of (a) doxorubicin, (b) poly(L-lysine iso-phthalamide) conjugated doxorubicin and (c) poly(L-lysine iso-phthalamide) (in its acid form, pre-dissolved in DMSO), in the presence of DMSO, was assessed in human breast cancer (MCF7) and mitoxantrone resistant (MCF7/MXR) cell lines.

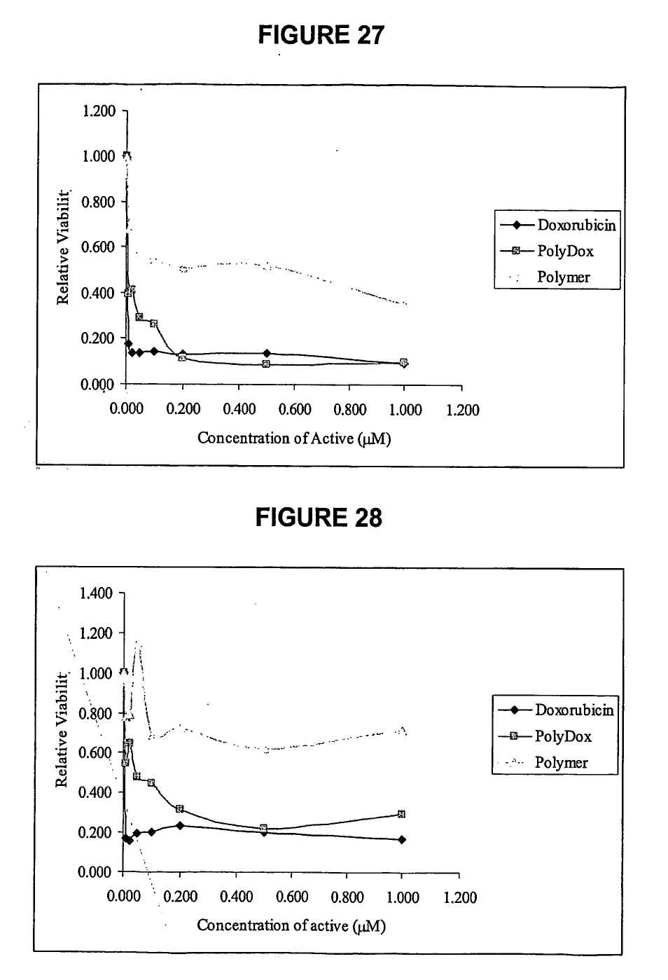

5×10³ cells of each type were seeded wells of a 96 well plate in 200 μL tissue culture media (10% foetal calf serum, 2 mM L-glutamine, 100 U/mL penicillin, and 100 μg/ml streptomycin) together with doxorubicin, or polymer-conjugated doxorubicin, or polymer alone (in its acid form, pre-dissolved in DMSO), at particular concentrations. The total DMSO concentration was adjusted to 1% by volume, and the cells incubated at 37° C. under a 5% CO₂ atmosphere for 5 days. The polymer had a molecular weight of approximately 18 kDa. Subsequently, a standard MTT assay was performed in order to assess cell viability. The results are illustrated in FIG. 27 (for MCF7 cells) and FIG. 28 (for MCF7/MXR cells).

The data demonstrate that, in the presence of 1% by volume of DMSO, both free doxorubicin and polymer-conjugated doxorubicin exhibit significant cytotoxic effects.

### REFERENCES

A number of patents and publications are cited above in order to more fully describe and disclose the invention and the state of the art to which the invention pertains. Full citations for these references are provided below. Each of these references is incorporated herein by reference in its entirety into the present disclosure, to the same extent as if each individual reference was specifically and individually indicated to be incorporated by reference.
Angelova, N., et al., 1999, “Rationalizing the design of polymeric biomaterials,” TIBTECH, October 1999, Vol. 17, pp. 409-421.
Anghel, D. F., et al., 1998, “Fluorescent dyes as model ‘hydrophobic modifiers’ of polyelectrolytes: a study of poly(acrylic acid)s labelled with pyrenyl and naphthyl groups,” Polymer, Vol. 39, No. 14, pp. 3035-3044.
Anufrieva, E. V., et al., 1968, “The models of denaturation of globular proteins II. Hydrophobic interactions and conformational transition in polymethacrylic acid,” J. Polymer Science: Part C, No. 16, pp. 3519-3531.
Atkins, P. W., 1989, Physical Chemistry, third edition, (publisher: Oxford University Press).
Ballou, B., et al., 1997, “Tumour visualisation using flurochrome-labeled antibodies,” Biotechnol. Progr., Vol. 13, pp. 640-658.
Boustta, M., et al., 1991, “New functional polyamides derived from citric acid and L-lysine: synthesis and characterization,” Makromol. Chem. Macromol. Symp., Vol. 47, pp. 345-355.
Coley M. C, Lewandowicz G, Sargent J. M and Verill M, 1997, “Chemosensitivity testing of fersh and continuous tumour cell cultures using lactate dehydrogenase,” Anticancer Research, Vol. 17, pp. 231-236.
Delaire, J. A.; et al., 1984, “Quenching of fluorescence in water-soluble copolymers of methacrylic acid and vinyldiphenylanthracene,” J. Phys. Chem., Vol. 88, pp. 6219-6227.
Domb, A. J., 1990, “Biodegradable polymers derived from amino acids,” Biomaterials, Vol. 11, pp. 686-689.
Dubin, P. L., et al., 1970, “Hydrophobic bonding in alternating copolymers of maleic acid and alkyl vinyl ethers,” J. Phys. Chem., Vol. 74, No. 14, pp. 2842-2847.
Eccleston M. E., et al., 1999, “Synthetic routes to responsive polymers: co-polycondensation of tri-functional amino acids with diacylchlorides,” Reactive and Functional Polymers, Vol. 42, pp. 147-161.
Eccleston, M. E. et al., 2000, “pH-Responsive Pseudo-Peptides for Cell Membrane Disruption,” J. Controlled Release, Vol. 69, p. 297-307.
Fiordeliso, J., et al., 1994, “Design, synthesis, and preliminary characterization of tyrosine-containing polyarylates: new biomaterials for medical applications,” J. Biomater. Sci. Polymer Edn., Vol. 5, No. 6, pp. 497-510.
Haensler, J., et al., 1993, “Polyamidoamine cascade polymers mediate efficient transfection of cells in culture,” Bioconjugate Chemistry, Vol. 4, pp. 372-379.
Hoffman, A. S., 1992, “Molecular bioengineering of biomaterials in the 1990s and beyond: a growing liaison of polymers with molecular biology,” Artificial Organs, Vol. 16, No. 1, pp. 43-49.
Hoffman, A. S., et al., 2000, “Really smart bioconjugates of smart polymers and receptor proteins,” Smart Bioconjugates (Founder's Award, Sixth World Biomaterials Congress 2000, Kamuela, Hi., May 15-20, 2000), pp. 577-586.
Jensen, K. D., et al., 2001, “The cytoplasmic escape and nuclear accumulation of endocytosed and microinjected HPMA copolymers and a basic kinetic study in Hep G2 cells,” American Association of Pharmaceutical Scientists, Vol. 3, No. 4, Article 32.
Kimoto, A., et al., 1992, “Antitumor effects of SMANCS on rat mammary tumor induced by 7,12-dimethylbenz[a]anthracene,” Cancer Research, Vol. 52, pp. 1013-1017.
Leyte, J. C. and Mandel, M., 1964, “Potentiometric Behaviour of Polymethacrylic acid”, J. Poly. Sci. Part. A, Vol. 2, pp. 1879-1891.
Li, C., et al., 1989, “Synthesis of poly(iminocarbonates): degradable polymers with potential applications as disposable plastics and as biomaterials,” Macromolecules, Vol. 22, No. 5, pp. 2029-2036.
Mandel et al., 1967, “The conformational transition of poly(methacrylic acid) in solution,” J. Phys. Chem., Vol. 71, No. 3, pp. 603-612.
Marecos, E., et al., 1998, “Antibody-mediated versus nontargeted delivery in a human small cell lung carcinoma model,” Bioconjugate Chem., Vol. 9, No. 2, pp. 184-191.
Matthews, S. E., et al., 1996, “Macromolecular systems for chemotherapy and magnetic resonance imaging,” Advanced Drug Delivery Reviews, Vol. 18, pp. 219-267.
Mungara, P. M., et al., 1993, “Synthesis of polyamides containing dipeptide linkages,” Chem. Mater., Vol. 5, No. 9, pp. 1242-1246.
Mungara, P. M., et al., 1994, “Chapter 11: Synthesis of Functionalized Targeted Polyamides,” in ACS Symp. Ser., Vol. 575, pp. 160-170.
Murthy, N., et al., 1999, “The design and synthesis of polymers for eukaryotic membrane disruption,” J. Controlled Release, Vol. 61, pp. 137-143.
Nagasawa, M., 1971, “Potentiometric titration and conformation of synthetic and natural polyelectrolytes,” Pure Appl. Chem., Vol. 26, Nos. 3-4, pp. 519-536.
Ohno, N., et al., 1973, “Conformational transition of the copolymer of maleic acid and styrene in aqueous solutions,” J. Polymer Science: Polymer Physics Edition, Vol. 11, pp. 413-425.
Ostolaza, H., et al., 1997, “Balance of electrostatic and hydrophobic interactions in the lysis of model membranes by E. coli α-haemolysin,” J. Membrane Biol., Vol. 158, pp. 137-145.
Plank, C., et al., 1995, “The influence of endosome-disruptive peptides on gene transfer using synthetic virus-like gene transfer systems,” J. Biol. Chem., Vol. 269, pp. 12918-12924.
Reddington, M. V., 1998, “New glycoconjugated cyanine dyes as fluorescence labelling reagents,” J. Chem. Soc., Perkin Trans., Vol. 1, pp. 144-147.
Reddington, M. V., 1998, “New glycosylated cyanine dyes as fluorescent labeling reagents,”. J. Chem. Soc., Perkin Trans. 1, pp. 143-148.
Rihova, B., et al., 2001, “Doxorubicin bound to a HPMA copolymer carrier through hydrazone bond is effective also in a cancer cell line with a limited content of lysosomes,” J. Control. Rel., Vol. 74, pp. 225-232.
Ringsdorf, H., 1975, “Structure and properties of pharmacologically active polymers,” J. Polymer Science: Symposium No. 51, pp. 135-153.
Rutkaite R., et al., 2001, “Fluorescence properties of carbazoyl-containing amphiphilic polymers,” J. Photochem. Photobiol. A: Chem., Vol. 138, pp. 245-251.
Rutkaite, R., et al., 2001, “Fluorescence properties of carbazolyl-containing amphiphilic copolymers,” J. Photochemistry and Photobiology A: Chemistry, Vol. 138, pp. 245-251.
Saotome, K., et al., 1967, “Optically active polyamides with regular structural sequences prepared from α-amino acid,” Die Makromolekulare Chemie, Vol. 109, pp. 239-248.
Seymour, L. W., et al., 1995, “Influence of molecular weight on passive tumour accumulation of a soluble macromolecular drug carrier,” European Journal of Cancer, Vol. 31A, No. 5, pp. 766-770.
Strying et al., 1989, in: Determination of Molecular Weight, Cooper A R, editor (Wiley, New York, pp. 263-300.
Thomas, J. L., et al., 1992, “Polyelectrolyte-sensitised phospholipid vesicles,” Acc. Chem. Res., Vol. 25, pp. 336-342.
Thomas, J. L., et al., 1994, “Membrane solubilization by a hydrophobic polyelectrolyte: surface activity and membrane binding,” Biophysical Journal, Vol. 67, pp. 1101-1106.
Thomas, J. L., et al., 1996, “Modulation of mobilities of fluorescent membrane probes by adsorption of a hydrophobic polyelectrolyte,” Macromolecules, Vol. 29, No. 7, pp. 2570-2572.
Tirrell, J. G., et al., 1996, “Synthesis of biopolymers: proteins, polyesters, polysaccharides and polynucleotides,” Current Opinion in Solid State & Materials Science, Vol. 1, pp. 407-411.
Weissleder, R., et al., 1999, “In vivo imaging of tumours with protease-actived near infrared fluorescent probes,” Nature Biotechnology, Vol. 17, pp. 375-378.
Yuan, P., et al., 1990, “pH-dependent fluorescence of merocyanine-eosin labeled water-soluble polymers,” Macromolecules, Vol. 23, pp. 4611-4615.

1-152. (canceled)

153. A method of delivering a payload into the nucleus of a living cell, comprising contacting the cell with a hypercoiling carrier polymer which incorporates, or is otherwise associated with, said payload, wherein said hypercoiling carrier polymer has both hydrophobic regions and hydrophilic regions.

154. A method according to claim 153, wherein said payload forms part of the backbone of said hypercoiling carrier polymer.

155. A method according to claim 153, wherein said payload is tethered to the backbone of said hypercoiling carrier polymer.

156. A method according to claim 153, wherein said hypercoiling carrier polymer is associated with said payload, and forms a complex with said payload.

157. A method according to claim 153, wherein the carrier polymer is biocompatible.

158. A method according to claim 153, wherein the carrier polymer is biodegradable.

159. A method according to claim 153, wherein the carrier polymer is not a vinyl polymer.

160. A method according to claim 153, wherein the carrier polymer has a backbone having amide linkages.

161. A method according to claim 153, wherein the carrier polymer has a backbone having ester linkages and amide linkages.

162. A method according to claim 153, wherein said hypercoiling carrier polymer has both hydrophobic regions and hydrophilic regions, and wherein said hydrophobic regions and hydrophilic regions alternate along the length of the backbone of the carrier polymer.

163. A method according to claim 153, wherein said carrier polymer has from 10 to 500 hydrophilic regions.

164. A method according to claim 153, wherein said carrier polymer has from 10 to 500 hydrophobic regions.

165. A method according to claim 153, wherein the ratio of hydrophilic regions to hydrophobic regions, by number, for the carrier polymer is from about 0.2 (1:5) to about 5 (5:1).

166. A method according to claim 153, wherein each hydrophobic region has a gram molecular weight of from about 14 to about 1000.

167. A method according to claim 153, wherein each hydrophilic region has a gram molecular weight of from about 16 to about 1000.

168. A method according to claim 153, wherein said carrier polymer and said payload have a combined molecular weight of from about 1 kDa to about 1 MDa.

169. A method according to claim 153, wherein exactly one, one or more, more than one, or all, of the hydrophobic moieties are selected from moieties derived from:
(a) an alkane, having from 1 to 20 carbon atoms;
(b) an alkene or an alkyne having from 2 to 20 carbon atoms;
(c) a cycloalkane, a cylcoalkene, or a cycloalkyne, having from 3 to 20 carbon atoms;
(d) a carboarene having from 6 to 20 ring carbon atoms;
(e) a heteroarene having from 5 to 20 ring atoms;
(f) a heterocycle having from 5 to 20 ring atoms;
(g) an alkane, an alkene, an alkyne, a cycloalkane, a cylcoalkene, or a cycloalkyne, as defined above, attached to a carboarene as defined above;
(h) an alkane, an alkene, an alkyne, a cycloalkane, a cylcoalkene, or a cycloalkyne, as defined above, attached to a heteroarene as defined above; or,
(i) an alkane, an alkene, an alkyne, a cycloalkane, a cylcoalkene, or a cycloalkyne, as defined above, attached to a heterocycle as defined above.

170. A method according to claim 153, wherein exactly one, one or more, more than one, or all, of the hydrophobic moieties are selected from:
moieties derived from compounds of the formula:
Q-G¹
wherein:
G¹ is a hydrophobic group; and
Q is independently a reactive functional group; and
moieties derived from compounds of the formula:
Q-G²-Q
wherein:
G² is a hydrophobic group; and
each Q is independently a reactive functional group;
wherein said hydrophobic group, G¹ or G², is selected from moieties derived from:
(a) an alkane, having from 1 to 20 carbon atoms;
(b) an alkene or an alkyne having from 2 to 20 carbon atoms;
(c) a cycloalkane, a cylcoalkene, or a cycloalkyne, having from 3 to 20 carbon atoms;
(d) a carboarene having from 6 to 20 ring carbon atoms;
(e) a heteroarene having from 5 to 20 ring atoms;
(f) a heterocycle having from 5 to 20 ring atoms;
(g) an alkane, an alkene, an alkyne, a cycloalkane, a cylcoalkene, or a cycloalkyne, as defined above, attached to a carboarene as defined above;
(h) an alkane, an alkene, an alkyne, a cycloalkane, a cylcoalkene, or a cycloalkyne, as defined above, attached to a heteroarene as defined above; or,
(i) an alkane, an alkene, an alkyne, a cycloalkane, a cylcoalkene, or a cycloalkyne, as defined above, attached to a heterocycle as defined above; and
said reactive functional group, Q, or each of said reactive functional groups, Q, is selected from:
(i) reactive acyl groups;
(ii) hydroxy groups; and,
(iii) amino groups; and
said reactive acyl group, —C(═O)X, is selected from:
(a) carboxylic acid, where X is —H;
(b) acyl halides, where X is halogen;
(c) acid anhydrides, where X is —OC(═O)R^{AN}, wherein R^{AN} is an acid anhydride substituent;
(d) esters, where X is —OR^{E}, wherein R^{E} is an ester substituent.

171. A method according to claim 170, wherein said hydrophobic group, G¹ or G², is derived from benzene.

172. A method according to claim 153, wherein exactly one, one or more, more than one, or all, of the hydrophobic moieties are selected from moieties derived from the following compounds:

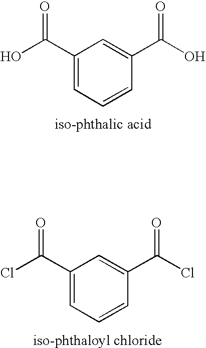

173. A method according to claim 170, wherein said hydrophobic group, G¹ or G², is derived from:
(a) an alkane, having from 1 to 20 carbon atoms.

174. A method according to claim 153, wherein exactly one, one or more, more than one, or all, of the hydrophobic moieties are selected from moieties derived from the following compounds, wherein p is an integer from 1 to 10:

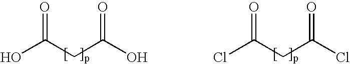

175. A method according to claim 153, wherein exactly one, one or more, more than one, or all, of the hydrophilic moieties is a weak Bronsted acid characterized by pKₐ values in the range of about 3 to about 8.

176. A method according to claim 153, wherein exactly one, one or more, more than one, or all, of the hydrophilic moieties is a weak Bronsted base characterized by pKₐ values in the range of about 5 to about 12.

177. A method according to claim 153, wherein exactly one, one or more, more than one, or all, of the hydrophilic moieties bears a carboxylic acid group or a salt thereof.

178. A method according to claim 153, wherein exactly one, one or more, more than one, or all, of the hydrophilic moieties bears an amino base group selected from:
a primary amino group (—NH₂);
a pendant secondary amino group (—NHR);
a non-pendant non-cyclic secondary amino group (—NH—);
a cyclic secondary amino group (—NH—);
a pendant tertiary amino group (—NR₂);
a non-pendant non-cyclic tertiary amino group (—NR— or —N═); or
a cyclic tertiary amino group (—NR— or —N═);
or a salt thereof.

179. A method according to claim 153, wherein exactly one, one or more, more than one, or all, of the hydrophilic moieties are selected from:
moieties derived from compounds of the formula: 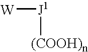
wherein:
J² is core group;
n is an integer from 1 to 4; and,
W is independently a reactive functional group; and
moieties derived from compounds of the formula: 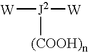
wherein:
J² is core group;
n is an integer from 1 to 4; and,
each W is independently a reactive functional group; and
wherein said core group, J¹ or J², is selected from moieties derived from:
(a) an alkane, having from 1 to 20 carbon atoms;
(b) an alkene or an alkyne having from 2 to 20 carbon atoms;
(c) a cycloalkane, a cylcoalkene, or a cycloalkyne, having from 3 to 20 carbon atoms;
(d) a carboarene having from 6 to 20 ring carbon atoms;
(e) a heteroarene having from 5 to 20 ring atoms;
(f) a heterocycle having from 5 to 20 ring atoms;
(g) an alkane, an alkene, an alkyne, a cycloalkane, a cylcoalkene, or a cycloalkyne, as defined above, attached to a carboarene as defined above;
(h) an alkane, an alkene, an alkyne, a cycloalkane, a cylcoalkene, or a cycloalkyne, as defined above, attached to a heteroarene as defined above; or,
(i) an alkane, an alkene, an alkyne, a cycloalkane, a cylcoalkene, or a cycloalkyne, as defined above, attached to a heterocycle as defined above; and
wherein said reactive functional group, W, or each of said reactive functional groups, W, is selected from:
(i) reactive acyl groups;
(ii) hydroxy; and,
(iii) amino groups.

180. A method according to claim 179, wherein J¹ and/or J² is independently a core group derived from an alkane having from 1 to 10 carbon atoms.

181. A method according to claim 153, wherein exactly one, one or more, more than one, or all, of the hydrophilic moieties are selected from moieties derived from amino acids.

182. A method according to claim 153, wherein exactly one, one or more, more than one, or all, of the hydrophilic moieties are selected from moieties derived from the following compounds: 2,4-diaminopropionic acid; 2,4-diaminobutyric acid; ornithine; lysine; 2,6-diaminopimelic acid.

183. A method according to claim 153, wherein one or more of the hydrophobic moieties of the carrier polymer are independently a hydrophobically-modified hydrophilic moiety.

184. A method according to claim 153, wherein the carrier polymer is a co-polymer of:
(a) a monomer selected from iso-phthalic acid and iso-phthaloyl chloride; and,
(b) a monomer selected from 2,4-diaminopropionic acid; 2,4-diaminobutyric acid; ornithine; lysine; or 2,6-diaminopimelic acid.

185. A method according to claim 184, wherein the carrier polymer is poly(lysine iso-phthalamide).

186. A method according to claim 153, wherein one or more of the payload moieties are, or comprise, biologically active agents selected from:
(a) drugs, prodrugs, chemo-therapeutics, radio-therapeutics, neutron capture agents;
(b) peptides, proteins, antibodies, antibody fragments, enzymes, transcription factors, signalling proteins, antisense peptides, zinc fingers, peptide vaccines; and,
(c) nucleotides, oligonucleotides, plasmids, nucleic acids.

187. A method according to claim 153, wherein one or more of the payload moieties are, or comprise, detectable labels selected from:
(a) fluorophores;
(b) chromophores;
(c) isotopically enriched species;
(d) paramagnetic species;
(e) radioactive species; and,
(f) scintillents and phosphors.

188. A method according to claim 153, wherein one or more of the payload moieties is a cyanine dye or a derivative thereof, a chelating group capable of complexing with a detectable label, a drug, or a boron-containing moiety, or one or more of the payload moieties is, or comprises, a peptide, a nucleic acid, or a cationic nucleic acid complex.

189. A method according to claim 153, wherein the carrier polymer further comprises other regions and/or moieties selected from: spacer groups, water solubilizing groups, and targeting ligands.

190. A method according to claim 153, wherein the carrier polymer further comprises water solubilizing groups selected from: polyethylene glycol (PEG), poly ethylene oxide (PEO), polyvinyl alcohol (PVA), hydroxylpropylmethyl alchohol (HPMA), and dextran groups.

191. A method of treatment of a condition comprising administering to a patient suffering from said condition a therapeutically-effective amount of a hypercoiling carrier polymer which incorporates a payload, or which is otherwise associated with a payload, as described in claim 153, wherein said payload is a drug which treats said condition.

192. A method of diagnosis of a condition comprising:
(a) administering to a patient an effective amount of a hypercoiling carrier polymer which incorporates a payload, or which is otherwise associated with a payload, as described in claim 153, wherein said payload is, or comprises, a detectable label;
(b) detecting the presence and/or location of said detectable label; and
(c) correlating said presence and/or location with said condition.

193. A method of imaging a cell comprising:
(a) contacting a living cell with a hypercoiling carrier polymer which incorporates a payload, or which is otherwise associated with a payload, as described in claim 153, further wherein said payload is, or comprises, a detectable label; and
(b) detecting the presence and/or location of said detectable label;
and optionally:
(c) forming an image of said cell using said presence and/or location data.

194. A method of imaging a patient, or a portion thereof, comprising:
(a) administering to said patient an effective amount of a hypercoiling carrier polymer which incorporates a payload, or which is otherwise associated with a payload, as described in claim 153, further wherein said payload is, or comprises, a detectable label; and
(b) detecting the presence and/or location of said detectable label; and optionally:
(c) forming an image of said patient, or portion thereof using said presence and/or location data.
